(19) United States
(12) Patent Application Publication
(10) Pub. No.: US 2014/0031257 A1
(43) Pub. Date: Jan. 30, 2014

(54) METHODS AND BIOMARKERS FOR DETECTION OF GASTROINTESTINAL CANCERS

(71) Applicants: Ragnhild A. Lothe, Oslo (NO); Guro E. Lind, Oslo (NO); Deeqa Ahmed, Oslo (NO); Kim Andresen, Oslo (NO); Rolf I. Skotheim, Oslo (NO)

(72) Inventors: Ragnhild A. Lothe, Oslo (NO); Guro E. Lind, Oslo (NO); Deeqa Ahmed, Oslo (NO); Kim Andresen, Oslo (NO); Rolf I. Skotheim, Oslo (NO)

(73) Assignee: OSLO UNIVERSITETSSYKEHUS HF, Nydalen (NO)

(21) Appl. No.: 14/001,779

(22) PCT Filed: Mar. 8, 2012
(86) PCT No.: PCT/IB12/00524

§ 371 Date: Sep. 27, 2013

Related U.S. Application Data
(60) Provisional application No. 61/451,198, filed on Mar. 10, 2011.

(51) Int. Cl. C12Q 1/68

(52) U.S. Cl. CPC C12Q 1/6886
USPC 506/9; 506/16

(57) ABSTRACT

The present invention relates to methods and biomarkers (e.g., epigenetic biomarkers) for detection of gastrointestinal cancers (e.g., colorectal cancer, gastric cancer, pancreatic cancer, liver cancer, cancer of the gall bladder and/or bile ducts (e.g., cholangiocarcinoma)) in biological samples (e.g., tissue samples, stool samples, blood samples, plasma samples, cell samples, gall samples, bile samples, serum samples).

### FIELD OF THE INVENTION

The present invention relates to methods and biomarkers (e.g., epigenetic biomarkers) for detection of gastrointestinal cancers (e.g., colorectal cancer, gastric cancer, pancreatic cancer, liver cancer, cancer of the gall bladder and/or bile ducts (e.g., cholangiocarcinoma)) in biological samples (e.g., tissue samples, stool samples, blood samples, plasma samples, cell samples, gall samples, bile samples, serum samples).

### BACKGROUND OF THE INVENTION

Cholangiocarcinoma (CCA) is the second most prevalent primary hepatobiliary malignancy and represents about 3% of all gastrointestinal cancers (1;2). According to the localization, CCAs are classified as being either extrahepatic or intrahepatic. Common for these subtypes is that they arise from the biliary epithelium and that they are difficult to diagnose. CCA is associated with inflammatory conditions in the biliary system, and patients with risk factors such as primary sclerosing cholangitis and liver fluke infestations have a higher risk of developing this malignancy (3-6). The generally late clinical presentation of CCA results in a high mortality, with a reported 5-year survival of only 5-15% (1;2). The diagnostics of CCA remains challenging. The current clinical strategy for rising a suspicion of malignancy includes a combination of various imaging modalities, as well as biliary brush cytology and analysis of a few serum markers (4;7;8). However, CCA can often not be confirmed until a laparoscopy has been performed (4). The most commonly used molecular marker for detecting CCA is carbohydrate antigen 19-9 (9). Unfortunately, this marker harbors limitations such as dependence of the Lewis phenotype and elevated levels due to the presence of other gastrointestinal malignancies or even benign conditions such as acute cholangitis and cirrhosis (10-12).

Tumor-specific molecular alterations, including both genetic- and epigenetic aberrations, have been shown to play important roles in cancer development (13-16). Impaired epigenetic regulation, including aberrant DNA methylation, is frequently reported in cancers (13; 16-18). In humans, DNA methylation occurs at the 5-position of cytosines in a CpG context (19). The bulk of the genome is methylated at the majority of these CpG sites, whereas dense CpG clusters, so-called CpG islands are usually devoid of methylation. Aberrant DNA hypermethylation of CpG islands located in the promoter region of genes is associated with transcriptional silencing (20;21). Loss of expression of essential tumor suppressor genes may lead to tumor development. Since aberrant DNA methylation also has been shown to be an early event in tumorigenesis (17; 22-24), such targets may represent attractive biomarkers for early detection. Several genes, including RASSF1A and CDKN2A (p16) have so far been analyzed for promoter methylation in CCA (Table 4). However, only genes frequently hypermethylated in tumors and unmethylated in normal tissue represent promising biomarkers. CCAs can only be cured by radical resection or in selected cases by liver transplantation. Most frequently, patients have too advanced disease at presentation to be candidates for surgery. The identification of suitable epigenetic CCA biomarkers with high sensitivity and specificity may facilitate cancer diagnostics at an early stage and thus contribute to increase survival of this patient cohort which presently holds poor outcome.

Colorectal cancer is one of the most common malignancies in the Western world, with an incidence of 3600 new cases per year in Norway alone. Colon cancer is cancer of the large intestine (colon), the lower part of the digestive system. Rectal cancer is cancer of the last several inches of the colon. Together, they're often referred to as colorectal cancers. Most cases of colon cancer begin as small, noncancerous (benign) clumps of cells called adenomatous polyps. Over time some of these polyps become colon cancers.

With early detection and treatment, colorectal cancers have a low mortality rate. However, later stage cancers require invasive treatments with unpleasant side effects and exhibit a much higher mortality rate.

Better, more effective non-invasive tests for early detection of cholangiocarcinoma, colorectal and other gastrointestinal cancers are needed to lower the morbidity and mortality associated with colorectal cancer.

### SUMMARY OF THE INVENTION

The present invention relates to methods and biomarkers (e.g., epigenetic biomarkers) for detection of gastrointestinal cancers (e.g., colorectal cancer, gastric cancer, pancreatic cancer, liver cancer, cancer of the gall bladder and/or bile ducts (e.g., cholangiocarcinoma)) in biological samples (e.g., tissue samples, stool samples, blood samples, plasma samples, cell samples, gall samples, bile samples, serum samples).

In experiments conducted during the course of developing some embodiments of the present invention, using a combined approach of microarray analysis and in vitro, in vivo and in silico analysis GLDC and PPP1R14A were identified as novel epigenetically deregulated genes in colorectal cancer. Both genes were unmethylated in normal mucosa samples, and frequently methylated in colorectal tumors, resulting in a sensitivity of 60% and 57%, respectively, and a specificity of 100%.

Further experiments identified several hypermethylated genes, where CDO1, DCLK1, ZNF331 isoform “c” and ZSCAN18 isoform “b” were found to be 100% methylated in bile duct- and colorectal cancer cell lines. The strict selection criteria applied throughout this approach along with the frequent promoter hypermethylation observed among primary tumours make CDO1, DCLK1, ZNF331 isoform “c” and ZSCAN18 isoform “b” useful biomarkers for early cancer detection.

For example, in some embodiments, the present invention provides a methylation specific nucleic acid detection sequence corresponding to one or more genes (e.g., GLDC, PPP1R14A, CDO1, DCLK1, ZSCAN18 or ZNF331). In some embodiments, the present invention provides a use of the nucleic acid sequence for detecting a cancerous condition in a subject (e.g., a gastrointestinal neoplasm; e.g., colorectal cancer). In some embodiments, the detection sequences are probes specific for one or more of GLDC, PPP1R14A, CDO1, DCLK1, ZSCAN18 and ZNF331 (SEQ ID NOs.: 185, 186, 187, 188, 189, and 190, respectively). In some embodiments, the detection sequences are primers specific for one or more of GLDC, PPP1R14A, CDO1, DCLK1, ZSCAN18 and ZNF331 (SEQ ID NOs.: 185, 186, 187, 188, 189, and 190, respectively). In some preferred embodiments, the methylation specific nucleic acid detection sequences are MSP (methylation-specific PCR) primers. Exemplary primer sets include, but are not limited to: for CDO1 (SEQ ID NO:17 and 18, SEQ ID NO:19 and 20, and SEQ ID NO:21 and 22); for DCLK1 (SEQ ID NO:39 and 40, SEQ ID NO:41 and 42, and SEQ ID NO:43 and 44); for ZSCAN18 (SEQ ID NO:161 and 162, SEQ ID NO:163 and 164, and SEQ ID NO:165 and 166). In some embodiments, the methylation specific nucleic acid detection sequence comprises a primer and probe set for quantitative MSP. Exemplary primer and probe set include, but are not limited to: for CDO1 (SEQ ID NOs.:170, 171 and 172); for DCLK1 (SEQ ID NOs:173, 174 and 175); for ZSCAN18 (SEQ ID NOs:179, 180 and 181); and for SFRP1 (176, 177 and 178). The detection sequences may be specific for regions upstream of the target gene, within the target gene, or downstream of the target gene. In some preferred embodiments, the detection sequences are specific for a region of the target sequence defined by a region that begins approximately −500, −400, −300, −200, −150, −100, −75 or −50 base pairs from the start codon of the target gene and ends at a position approximately −10, −10, 0, 10, 20, 30, 40, 50, 100, 120, 150 or 200 base pairs from the start codon of the target gene.

Further embodiments of the present invention provide a method for detecting a gastrointestinal neoplasm in a subject comprising: obtaining DNA from a biological sample of the subject; and determining the level, presence, or frequency of methylation of nucleic acid polymers corresponding to one or more genes (e.g., GLDC, PPP1R14A, CDO1, DCLK1, ZSCAN18 or ZNF331). In some embodiments, the nucleic acid comprises a CpG island or a CpG island shore. In some embodiments, the CpG island or shore is present in a coding region or a regulatory region (e.g., a promoter). In some embodiments, determining of the level of altered methylation of a nucleic acid polymer comprises determining the methylation frequency of the CpG island or island shore. In some embodiments, determining of the level of a nucleic acid polymer with altered methylation is achieved by a technique selected from, for example, methylation-specific PCR, quantitative methylation-specific PCR, methylation-sensitive DNA restriction enzyme analysis, quantitative bisulfite pyrosequencing, next generation sequencing and bisulfite genomic sequencing PCR. In some embodiments, the methods employ the methylation specific nucleic acid detection sequences described in detail above. In some embodiments, the method further comprises generating a risk profile. In some embodiments, the gastrointestinal neoplasm is colorectal cancer. In some embodiments, the method permits detection of gastrointestinal cancer in the subject with a sensitivity of at least 85% at a specificity of at least 85%. In other embodiments, the method permits detection of gastrointestinal cancer in the subject with a sensitivity of at least 80% at a specificity of at least 90%. In some embodiments, the biological sample is a tissue samples, a stool sample, a blood sample, a plasma sample, a cell sample, a gall sample, a bile sample or a serum sample.

Additional embodiments of the present invention provide a kit for detecting the presence of a gastrointestinal neoplasm in a mammal, the kit comprising reagents useful, sufficient, or necessary for detecting and/or characterizing level, presence, or frequency of methylation of one or more genes selected from, for example, GLDC, PPP1R14A, CDO1, DCLK1, ZSCAN18 and ZNF331. In some embodiments, the kits employ the methylation specific nucleic acid detection sequences described in detail above.

Additional embodiments will be apparent to persons skilled in the relevant art based on the teachings contained herein.

### DESCRIPTION OF THE DRAWINGS

FIG. 1. Strategy to select novel DNA methylation candidate genes.

FIG. 2. ROC curve analysis from quantitative methylation-specific PCR results of GLDC.

FIG. 3. Roc curve analysis from quantitative methylation-specific PCR results of PPP1R14A.

FIG. 4. Comparison of scores obtained from conventional MSP and quantitative MSP analysis of GLDC.

FIG. 5. Comparison of scores obtained from conventional MSP and quantitative MSP of PPP1R14A.

FIG. 6. Bisulfite sequencing verifies site specific methylation within the GLDC promoter. A) The upper part of the figure is a schematic presentation of the CpG sites amplified by bisulfite sequencing primers. B) Representative bisulfite sequencing electropherograms of the GLDC promoter in colon cancer cell lines.

FIG. 7. Bisulfite sequencing verifies site specific methylation within the PPP1R14A promoter. A) The upper part of the figure is a schematic presentation of the CpG sites amplified by bisulfite sequencing primers. B) Representative bisulfite sequencing electropherograms of the PPP1R14A promoter in colon cancer cell lines.

FIG. 8. Epigenome-wide experimental approach for identifying hypermethylated genes in cholangiocarcinomas. Six CCA cell lines were cultured with and without a combination of epigenetic drugs (5-aza-2″deoxycytidine and trichostatin A). Array-elements responding to epigenetic drug treatment were compared to previously published down-regulated genes in CCAs compared to cancer free tissue. Common genes, harboring a CpG island in the promoter region, were investigated for hypermethylation in cancer cell lines from colon, bile duct, liver, gall bladder, and pancreas. Genes frequently methylated in CCA cell lines were subsequently investigated in patient material using MSP. The most promising candidates from this analysis were further evaluated by qMSP. Numbers indicate the number of genes fulfilling the selection criteria in each experimental step and subsequently subjected to further analyses.

FIG. 9. Sequences of exemplary target genes.

FIG. 10: Venn diagram illustrating overlapping de-regulated genes between cancer cell lines and cholangiocarcinomas. Using microarray analyses, sixty genes were identified as up-regulated in CCA cell lines after epigenetic drug treatment, and simultaneously down-regulated in tumor compared to non-malignant controls in previously published datasets. Abbreviations: ICC, intrahepatic cholangiocarcinoma; ECC, extrahepatic cholangiocarcinoma.

FIG. 11: Summary of promoter methylation status in cancer cell lines. Forty loci were analyzed by MSP in cancer cell lines and grouped according to their methylation frequency in CCA cell lines. Group I; frequently methylated (minimum five out of six cell lines), group II; intermediately methylated (from one to four cell lines), group III; unmethylated.

FIG. 12: Direct bisulfite sequencing of CDO1, DCLK1 and ZSCAN18 verified the methylation status as assessed by MSP. A) CDO1. B) DCLK1. C) ZSCAN18. For all panels, the upper line represents the individual CpG sites (vertical bars) in the fragment amplified by the bisulfite sequencing primers. Transcription start site is denoted by +1 and arrows indicate the location of MSP and the subsequently designed qMSP primers and probe. In the lower part of the panels, dark circles indicate methylated CpGs, grey circles indicate partially methylated CpGs, and white circles indicate unmethylated CpGs. The column on the right side in each panel (M, U/M and U) lists the methylation status as assessed by MSP.

FIG. 13: Receiver operating characteristics curves for individual and combined genes in cholangiocarcinomas and non-malignant samples. The panels depict the resulting area under the receiver operating characteristics curve values based on the PMR for the individual biomarkers in the A) fresh frozen sample series, B) archival sample series and C) combined for fresh frozen and archival material. D-F shows the performance of the combined biomarker panel in D) fresh frozen-, E) archival- and F) combined sample sets.

FIG. 14: Methylation frequencies in patient material assessed by qualitative methylation-specific polymerase chain reaction (qMSP). The twelve group I genes were investigated in a fresh frozen sample set. Band intensities from the methylated reaction were considerably weaker in non-malignant samples than in carcinomas.

### DEFINITIONS

To facilitate an understanding of the present invention, a number of terms and phrases are defined below:

As used herein, the term “sensitivity” is defined as a statistical measure of performance of an assay (e.g., method, test), calculated by dividing the number of true positives by the sum of the true positives and the false negatives.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “CpG island” refers to a genomic DNA region that contains a high percentage of CpG sites relative to the average genomic CpG incidence (per same species, per same individual, or per subpopulation (e.g., strain, ethnic subpopulation, or the like). Various parameters and definitions for CpG islands exist; for example, in some embodiments, CpG islands are defined as having a GC percentage that is greater than 50% and with an observed/expected CpG ratio that is greater than 60% (Gardiner-Garden et al. (1987) J Mol. Biol. 196:261-282; Baylin et al. (2006) Nat. Rev. Cancer 6:107-116; Irizarry et al. (2009) Nat. Genetics 41:178-186; each herein incorporated by reference in its entirety). In some embodiments, CpG islands may have a GC content >55% and observed CpG/expected CpG of 0.65 (Takai et al. (2007) PNAS 99:3740-3745; herein incorporated by reference in its entirety). Various parameters also exist regarding the length of CpG islands. As used herein, CpG islands may be less than 100 bp; 100-200 bp, 200-300 bp, 300-500 bp, 500-750 bp; 750-1000 bp; 100 or more by in length. In some embodiments, CpG islands show altered methylation patterns relative to controls (e.g., altered methylation in cancer subjects relative to subjects without cancer; tissue-specific altered methylation patterns; altered methylation in biological samples (e.g., tissue, stool, blood, plasma, serum, cells, bile) from subjects with gastrointestinal neoplasia (e.g., colorectal cancer, gastric cancer, pancreatic cancer, liver cancer, cancers of the gall bladder and/or bile ducts (e.g., cholangiocarcinoma)) relative to subjects without gastrointestinal neoplasia). In some embodiments, altered methylation involves hypermethylation. In some embodiments, altered methylation involves hypomethylation.

As used herein, the term “CpG shore” or “CpG island shore” refers to a genomic region external to a CpG island that is or that has potential to have altered methylation patterns (see, e.g., Irizarry et al. (2009) Nat. Genetics 41:178-186; herein incorporated by reference in its entirety). CpG island shores may show altered methylation patterns relative to controls (e.g., altered methylation in cancer subjects relative to subjects without cancer; tissue-specific altered methylation patterns; altered methylation in biological samples (e.g., stool, tissue, blood, cells, bile) from subjects with gastrointestinal neoplasia (e.g., colorectal cancer, gastric cancer, pancreatic cancer, liver cancer, cancers of the gall bladder and/or bile ducts (e.g., cholangiocarcinoma)) relative to subjects without gastrointestinal neoplasia). In some embodiments, altered methylation involves hypermethylation. In some embodiments, altered methylation involves hypomethylation. CpG island shores may be located in various regions relative to CpG islands (see, e.g., Irizarry et al. (2009) Nat. Genetics 41; 178-186; herein incorporated by reference in its entirety). Accordingly, in some embodiments, CpG island shores are located less than 100 bp; 100-250 bp; 250-500 bp; 500-1000 bp; 1000-1500 bp; 1500-2000 bp; 2000-3000 bp; 3000 bp or more away from a CpG island.

As used herein, the term “epigenetic” refers to a non-sequence-based alteration that is inherited through cell division. For example, in some embodiments, epigenetic changes are altered methylation patters or levels (e.g. hypermethylation).

As used herein the term “methylation state” is a measure of the presence or absence of a methyl modification in one or more CpG sites in at least one nucleic acid sequence. It is to be understood that in some embodiments, the methylation state of one or more CpG sites is determined in multiple copies of a particular gene of interest.

As used herein, the term “methylation level” refers to the amount of methylation in one or more copies of a gene or nucleic acid sequence of interest. The methylation level may be calculated as an absolute measure of methylation within the gene or nucleic acid sequence of interest. Also a “relative methylation level” may be determined as the amount of methylated DNA, relative to the total amount DNA present or as the number of methylated copies of a gene or nucleic acid sequence of interest, relative to the total number of copies of the gene or nucleic acid sequence. Additionally, the “methylation level” can be determined as the percentage of methylated CpG sites within the DNA stretch of interest.

The term methylation level also encompasses the situation wherein one or more CpG site in e.g. the promoter region is methylated but where the amount of methylation is below amplification threshold. Thus methylation level may be an estimated value of the amount of methylation in a gene of interest.

In some embodiments, the methylation level of the gene of interest is 15% to 100%, such as 50% to 100%, more preferably 60%-100%, more preferably 70-100%, more preferably 80% to 100%, more preferably 90% to 100%. Thus in one embodiment of the present invention the methylation level of the genes according to the invention is 80%, 81%, 82%, 83%, 84%, 85%, 86%, 87%, 88%, 89%, 90%, 91%, 92%, 93%, 94%, 95%, 96%, 97%, 98%, 99% or 100%.

As used herein, the term “methylation specific nucleic acid detection sequence” refers to a probe, probes or primers or sets thereof that are used to specifically detect, determine or analyze the methylation status of a target nucleic acid sequence, e.g., the sequences encoding GLDC, PPP1R14A, CDO1, DCLK1, ZSCAN18 and ZNF331 (SEQ ID NOs.: 185, 186, 187, 188, 189, and 190, respectively). The detection sequence may be a single probe or comprise multiple probes, such as would be the case for a set of PCR primers specific for the target sequence. Specific examples of “methylation specific nucleic acid detection sequences” include, but are not limited to, MSP and qMSP primer sets.

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body. As used herein, the term “metastasized gastrointestinal cancer cells” is meant to refer to gastrointestinal cancer cells that have metastasized; gastrointestinal cancer cells localized in a part of the body other than the gastrointestinal system.

As used herein, “an individual is suspected of being susceptible to metastasized gastrointestinal cancer” is meant to refer to an individual who is at an above-average risk of developing metastasized gastrointestinal cancer. Examples of individuals at a particular risk of developing gastrointestinal cancer are those whose family medical history indicates above average incidence of gastrointestinal cancer among family members and/or those who have already developed gastrointestinal cancer and have been effectively treated who therefore face a risk of relapse and recurrence. Other factors which may contribute to an above-average risk of developing metastasized gastrointestinal cancer which would thereby lead to the classification of an individual as being suspected of being susceptible to metastasized gastrointestinal cancer may be based upon an individual's specific genetic, medical and/or behavioral background and characteristics.

The term “neoplasm” as used herein refers to any new and abnormal growth of tissue. Thus, a neoplasm can be a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells. The term “aero-digestive system neoplasm-specific marker” refers to any biological material that can be used to indicate the presence of a gastrointestinal system neoplasm (e.g., a premalignant gastrointestinal system neoplasm; a malignant gastrointestinal system neoplasm). Examples of gastrointestinal system-specific markers include, but are not limited to, GLDC, PPP1R14A, CDO1, DCLK1, ZSCAN18 and ZNF331.

As used herein, the term “amplicon” refers to a nucleic acid generated using primer pairs. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

A “subsequence” is any portion of an entire sequence. Thus, a subsequence refers to a consecutive sequence of amino acids or nucleic acids which is part of a longer sequence of nucleic acids (e.g. polynucleotide).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences”. Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) processed transcript. The mRNA functions during translation to specify the sequence or order of amino acids in a nascent polypeptide.

The term “locus” as used herein refers to a nucleic acid sequence on a chromosome or on a linkage map and includes the coding sequence as well as 5′ and 3′ sequences involved in regulation of the gene.

### DETAILED DESCRIPTION OF THE INVENTION

The present invention relates to methods and biomarkers (e.g., epigenetic biomarkers) for detection of gastrointestinal cancers (e.g., colorectal cancer, gastric cancer, pancreatic cancer, liver cancer, cancers of the gall bladder and/or bile ducts (e.g., cholangiocarcinoma)) in biological samples (e.g., tissue samples, stool samples, blood samples, plasma samples, serum samples, cell samples, gall samples, bile samples).

Impaired epigenetic regulation is as common as gene mutations in human cancer. These mechanisms lead to quantitative and qualitative gene expression changes causing a selective growth advantage, which may result in cancerous transformation. Aberrantly hypermethylated CpG islands in the gene promoter associated with transcriptional inactivation are among the most frequent epigenetic changes in cancer. Since early detection of disease can result in improved clinical outcome for most types of cancer, the identification of cancer-associated aberrant gene methylation represents promising novel biomarkers. For cancers in the gastrointestinal system, including, e.g., colorectal cancer, gastric cancer, pancreatic cancer, liver cancer, cancers of the gall bladder and/or bile ducts (e.g., cholangiocarcinoma), initial studies have identified the presence of aberrantly methylated DNA in patient blood and feces. Genes aberrantly hypermethylated in high frequencies already among benign tumours and only rarely in normal mucosa would be good candidate diagnostic biomarkers due to the potential clinical benefit of early detection of high risk adenomas as well as of low risk stages of carcinomas.

In general, however, the sensitivity and specificity of existing early markers for cancers in the gastrointestinal system remain poor. Consequently, there is a need for individual genes or a panel of genes in which each gene is hypermethylated at a high frequency and specificity in cancers. In particular, there is a need for a gene panel which is useful in non-invasive techniques, such as techniques involving the use of stool samples, or in techniques which may be used on sample material which is easily obtained, such as blood or blood products or mucous. Such a gene panel would greatly improve the possibility for early detection of these cancers.

Accordingly, embodiments of the present invention provide compositions and methods comprising detection of the methylation status of one or more of GLDC, PPP1R14A, CDO1, DCLK1, ZSCAN18 and ZNF331. The compositions and methods find use in research, screening and clinical applications (e.g., related to colorectal cancer, gastric cancer, pancreatic cancer, liver cancer, cancers of the gall bladder and/or bile ducts (e.g., cholangiocarcinoma)).

Therefore, using a highly sensitive automated and quantitative screening methodology for detecting cancer-related promoter methylation, novel epi-biomarkers and epi-biomarker panels were identified that are frequently and specifically methylated in gastrointestinal cancer.

While the present invention exemplifies several markers specific for detecting gastrointestinal cancer, any marker that is correlated with the presence or absence of gastrointestinal cancer may be used. A marker, as used herein, includes, for example, nucleic acid(s) whose production or mutation or lack of production is characteristic of a gastrointestinal neoplasm. Depending on the particular set of markers employed in a given analysis, the statistical analysis will vary. For example, where a particular combination of markers is highly specific for gastrointestinal cancer, the statistical significance of a positive result will be high. It may be, however, that such specificity is achieved at the cost of sensitivity (e.g., a negative result may occur even in the presence of gastrointestinal cancer). By the same token, a different combination may be very sensitive (e.g., few false negatives, but has a lower specificity).

Particular combinations of markers may be used that show optimal function with different ethnic groups or sex, different geographic distributions, different stages of disease, different degrees of specificity or different degrees of sensitivity. Particular combinations may also be developed which are particularly sensitive to the effect of therapeutic regimens on disease progression. Subjects may be monitored after a therapy and/or course of action to determine the effectiveness of that specific therapy and/or course of action.

The methods of the present invention are not limited to particular indicators of gastrointestinal neoplasm. In some embodiments, indicators of gastrointestinal neoplasm include, for example, epigenic alterations. Epigenetic alterations include but are not limited to DNA methylation (e.g., CpG methylation). In some embodiments, the level (e.g., frequency, score) of methylation (e.g., hypermethylation relative to a control, hypomethylation relative to a control) is determined without limitation to the technique used for such determining. Methods of the present invention are not limited to particular epigenetic alterations (e.g., DNA methylation) (e.g., CpG methylation) (e.g., CpG methylation in coding or regulatory regions of GLDC, PPP1R14A, CDO1, DCLK1, ZSCAN18 or ZNF331). Altered methylation may occur in, for example, CpG islands; CpG island shores; or regions other than CpG islands or CpG island shores.

In certain embodiments, methods, kits, and systems of the present invention involve determination of methylation state of a locus of interest (e.g., in human DNA) (e.g., in human DNA extracted from a stool sample, from a gastrointestinal tissue sample, from a tumor sample, from a blood sample, from a serum sample, from a plasma sample, from a cell sample, from a bile sample, etc). Any appropriate method can be used to determine whether a particular DNA is hypermethylated or hypomethylated. Standard PCR techniques, for example, can be used to determine which residues are methylated, since unmethylated cytosines converted to uracil are replaced by thymidine residues during PCR. PCR reactions can contain, for example, 10 μL of captured DNA that either has or has not been treated with sodium bisulfite, IX PCR buffer, 0.2 mM dNTPs, 0.5 μM sequence specific primers (e.g., primers flanking a CpG island or CpG shore within the captured DNA), and 5 units DNA polymerase (e.g., Amplitaq DNA polymerase from PE Applied Biosystems, Norwalk, Conn.) in a total volume of 50 μl. A typical PCR protocol can include, for example, an initial denaturation step at 94° C. for 5 min, 40 amplification cycles consisting of 1 minute at 94° C., 1 minute at 60° C., and 1 minute at 72° C., and a final extension step at 72° C. for 5 minutes.

To analyze which residues within a captured DNA are methylated, the sequences of PCR products corresponding to samples treated with and without sodium bisulfite can be compared. The sequence from the untreated DNA will reveal the positions of all cytosine residues within the PCR product. Cytosines that were unmethylated will be converted to thymidine residues in the sequence of the bisulfite-treated DNA, while residues that were methylated will be unaffected by bisulfite treatment.

Some embodiments of the present invention utilize next generation or high-throughput sequencing. A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Similarly, in some embodiments, methods of the present invention involve the determination (e.g., assessment, ascertaining, quantitation) of methylation level of an indicator of gastrointestinal neoplasm (e.g., the methylation level of a CpG island or CpG shore in the coding or regulatory region of a gene locus) in a sample (e.g., a DNA sample extracted from stool, bile or blood). A skilled artisan understands that an increased, decreased, informative, or otherwise distinguishably different methylation level is articulated with respect to a reference (e.g., a reference level, a control level, a threshold level, or the like). For example, the term “elevated methylation” as used herein with respect to the methylation status (e.g., CpG DNA methylation) of a gene locus (e.g., GLDC, PPP1R14A, CDO1, DCLK1, ZSCAN18 or ZNF331) is any methylation level that is above a median methylation level in a sample from a random population of mammals (e.g., a random population of 10, 20, 30, 40, 50, 100, or 500 mammals) that do not have a gastrointestinal neoplasm (e.g., gastrointestinal cancer). Elevated levels of methylation can be any level provided that the level is greater than a corresponding reference level. For example, an elevated methylation level of a locus of interest (e.g., GLDC, PPP1R14A, CDO1, DCLK1, ZSCAN18 or ZNF331) methylation can be 0.5, 1, 2, 3, 4, 5, 6, 7, 8, 9, 10, or more fold greater than the reference level methylation observed in a normal sample. It is noted that a reference level can be any amount. The term “elevated methylation score” as used herein with respect to detected methylation events in a matrix panel of particular nucleic acid markers is any methylation score that is above a median methylation score in a sample from a random population of mammals (e.g., a random population of 10, 20, 30, 40, 50, 100, or 500 mammals) that do not have a gastrointestinal neoplasm (e.g., colorectal cancer or cholangiocarcinoma). An elevated methylation score in a matrix panel of particular nucleic acid markers can be any score provided that the score is greater than a corresponding reference score. For example, an elevated score of methylation in a locus of interest (e.g., GLDC, PPP1R14A, CDO1, DCLK1, ZSCAN18 or ZNF331) can be 0.5, 1, 2, 3, 4, 5, 6, 7, 8, 9, 10, or more fold greater than the reference methylation score observed in a normal sample. It is noted that a reference score can be any amount.

The methods are not limited to a particular type of mammal. In some embodiments, the mammal is a human. In some embodiments, the gastrointestinal neoplasm is premalignant. In some embodiments, the gastrointestinal neoplasm is malignant. In some embodiments, the gastrointestinal neoplasm is gastrointestinal (e.g., colorectal cancer, gastric cancer, pancreatic cancer, liver cancer, cancers of the gall bladder and/or bile ducts (e.g., cholangiocarcinoma)) without regard to stage of the cancer (e.g., stage I, II, III, or IV).

The present invention also provides methods and materials to assist medical or research professionals in determining whether or not a mammal has a gastrointestinal neoplasm (e.g., colorectal cancer, gastric cancer, pancreatic cancer, liver cancer, cancers of the gall bladder and/or bile ducts (e.g., cholangiocarcinoma)). Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the ratio of particular markers in a sample, and (2) communicating information about the ratio to that professional, for example.

After the level (score, frequency) of particular markers in a stool, blood, serum, bile or plasma sample is reported, a medical professional can take one or more actions that can affect patient care. For example, a medical professional can record the results in a patient's medical record. In some cases, a medical professional can record a diagnosis of a gastrointestinal neoplasia, or otherwise transform the patient's medical record, to reflect the patient's medical condition. In some cases, a medical professional can review and evaluate a patient's entire medical record, and assess multiple treatment strategies, for clinical intervention of a patient's condition. In some cases, a medical professional can record a prediction of tumor occurrence with the reported indicators. In some cases, a medical professional can review and evaluate a patient's entire medical record and assess multiple treatment strategies, for clinical intervention of a patient's condition.

A medical professional can initiate or modify treatment of a gastrointestinal neoplasm after receiving information regarding the level (score, frequency) associated with markers in a patient's stool, blood, serum, bile or plasma sample. In some cases, a medical professional can compare previous reports and the recently communicated level (score, frequency) of markers, and recommend a change in therapy. In some cases, a medical professional can enroll a patient in a clinical trial for novel therapeutic intervention of gastrointestinal neoplasm. In some cases, a medical professional can elect waiting to begin therapy until the patient's symptoms require clinical intervention.

A medical professional can communicate the assay results to a patient or a patient's family. In some cases, a medical professional can provide a patient and/or a patient's family with information regarding gastrointestinal neoplasia, including treatment options, prognosis, and referrals to specialists, e.g., oncologists and/or radiologists. In some cases, a medical professional can provide a copy of a patient's medical records to communicate assay results to a specialist. A research professional can apply information regarding a subject's assay results to advance gastrointestinal neoplasm research. For example, a researcher can compile data on the assay results, with information regarding the efficacy of a drug for treatment of gastrointestinal neoplasia to identify an effective treatment. In some cases, a research professional can obtain assay results to evaluate a subject's enrollment, or continued participation in a research study or clinical trial. In some cases, a research professional can classify the severity of a subject's condition, based on assay results. In some cases, a research professional can communicate a subject's assay results to a medical professional. In some cases, a research professional can refer a subject to a medical professional for clinical assessment of gastrointestinal neoplasia, and treatment thereof. Any appropriate method can be used to communicate information to another person (e.g., a professional). For example, information can be given directly or indirectly to a professional. For example, a laboratory technician can input the assay results into a computer-based record. In some cases, information is communicated by making a physical alteration to medical or research records. For example, a medical professional can make a permanent notation or flag a medical record for communicating a diagnosis to other medical professionals reviewing the record. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

It is noted that a single sample can be analyzed for one gastrointestinal neoplasm-specific marker or for multiple gastrointestinal neoplasm-specific markers. In preferred embodiments, a single sample is analyzed for multiple gastrointestinal neoplasm-specific markers, for example, using multi-marker assays. In addition, multiple samples can be collected for a single mammal and analyzed as described herein. In some embodiments, a sample is split into first and second portions, where the first portion undergoes cytological analysis and the second portion undergoes further purification or processing (e.g., sequence-specific capture step(s) (e.g., for isolation of specific markers for analysis of methylation levels). In some embodiments, the sample undergoes one or more preprocessing steps before being split into portions. In some embodiments, the sample is treated, handled, or preserved in a manner that promotes DNA integrity and/or inhibits DNA degradation (e.g., through use of storage buffers with stabilizing agents (e.g., chelating agents, DNase inhibitors) or handling or processing techniques that promote DNA integrity (e.g., immediate processing or storage at low temperature (e.g., −80 degrees C.)).

Some embodiments of the invention provides a diagnostic kit for the diagnosis or screening of cancer comprising one or reagents for detection of methylation status of the genes selected from, for example one or more of GLDC, PPP1R14A, CDO1, DCLK1, ZSCAN18 or ZNF331. For example, in some embodiments, the reagents comprise nucleic acids (e.g., oligonucleotides, primers, probes, etc.). In some embodiments, kits provide reagents useful, necessary or sufficient for detecting methylation status and/or providing a diagnosis or prognosis.

The diagnostic kits may further comprise any reagent or media necessary, sufficient or useful to perform analyses, such as PCR analyses, such as methylation specific polymerase chain reaction (MSP) sequence analyses, bisulphite treatment, bisulphite sequencing, electrophoresis, pyrosequencing, mass spectrometry and sequence analyses by restriction digestion, next generation sequencing, quantitative and/or qualitative methylation, pyrosequencing, Southern blotting, restriction landmark genome scanning (RLGS), single nucleotide primer extension, CpG island microarray, SNUPE, COBRA, mass spectrometry, by use of methylation specific restriction enzymes or by measuring the expression level of said genes. In particular, the kit may further comprise one or more components selected from the group consisting of: deoxyribonucleoside triphosphates, buffers, stabilizers, thermostable DNA polymerases, restriction endonucleases (including methylation specific endonucleases), and labels (including fluorescent, chemiluminescent and radioactive labels). The diagnostic assay according to the invention may further comprise one or more reagents required for isolation of DNA.

In some embodiments, the kits of the present invention include a means for containing the reagents in close confinement for commercial sale such as, e.g., injection or blow-molded plastic containers into which the desired reagent are retained. Other containers suitable for conducting certain steps of the disclosed methods also may be provided.

In some embodiments, the methods disclosed herein are useful in monitoring the treatment of gastrointestinal neoplasia (e.g., colorectal cancer, gastric cancer, pancreatic cancer, liver cancer, cancers of the gall bladder and/or bile ducts (e.g., cholangiocarcinoma)). For example, in some embodiments, the methods may be performed immediately before, during and/or after a treatment to monitor treatment success. In some embodiments, the methods are performed at intervals on disease free patients to ensure treatment success.

The present invention also provides a variety of computer-related embodiments. Specifically, in some embodiments the invention provides computer programming for analyzing and comparing a pattern of gastrointestinal neoplasm-specific marker detection results in a sample obtained from a subject to, for example, a library of such marker patterns known to be indicative of the presence or absence of a gastrointestinal neoplasm, or a particular stage or gastrointestinal neoplasm.

In some embodiments, the present invention provides computer programming for analyzing and comparing a first and a second pattern of gastrointestinal neoplasm-specific marker detection results from a sample taken at least two different time points. In some embodiments, the first pattern may be indicative of a pre-cancerous condition and/or low risk condition for gastrointestinal cancer and/or progression from a pre-cancerous condition to a cancerous condition. In such embodiments, the comparing provides for monitoring of the progression of the condition from the first time point to the second time point.

In yet another embodiment, the invention provides computer programming for analyzing and comparing a pattern of gastrointestinal neoplasm-specific marker detection results from a sample to a library of gastrointestinal neoplasm-specific marker patterns known to be indicative of the presence or absence of a gastrointestinal cancer, wherein the comparing provides, for example, a differential diagnosis between a benign gastrointestinal neoplasm, and an aggressively malignant gastrointestinal neoplasm (e.g., the marker pattern provides for staging and/or grading of the cancerous condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, distributed servers (e.g., as used in cloud computing) or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., specific gene products assayed), and test result data (e.g., the pattern of gastrointestinal neoplasm-specific marker detection results from a sample). This information received can be stored at least temporarily in a database, and data analyzed in comparison to a library of marker patterns known to be indicative of the presence or absence of a pre-cancerous condition, or known to be indicative of a stage and/or grade of gastrointestinal cancer.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In some embodiments, all or a portion of the input data and/or all or a portion of the output data (e.g., usually at least the library of the pattern of gastrointestinal neoplasm-specific marker detection results known to be indicative of the presence or absence of a pre-cancerous condition) are maintained on a server for access, e.g., confidential access. The results may be accessed or sent to professionals as desired.

A system for use in the methods described herein generally includes at least one computer processor (e.g., where the method is carried out in its entirety at a single site) or at least two networked computer processors (e.g., where detected marker data for a sample obtained from a subject is to be input by a user (e.g., a technician or someone performing the assays)) and transmitted to a remote site to a second computer processor for analysis (e.g., where the pattern of gastrointestinal neoplasm-specific marker) detection results is compared to a library of patterns known to be indicative of the presence or absence of a pre-cancerous condition), where the first and second computer processors are connected by a network, e.g., via an intranet or internet). The system can also include a user component(s) for input; and a reviewer component(s) for review of data, and generation of reports, including detection of a pre-cancerous condition, staging and/or grading of a gastrointestinal neoplasm, or monitoring the progression of a pre-cancerous condition or a gastrointestinal neoplasm. Additional components of the system can include a server component(s); and a database(s) for storing data (e.g., as in a database of report elements, e.g., a library of marker patterns known to be indicative of the presence or absence of a pre-cancerous condition and/or known to be indicative of a grade and/or a stage of a gastrointestinal neoplasm, or a relational database (RDB) which can include data input by the user and data output. The computer processors can be processors that are typically found in personal desktop computers (e.g., IBM, Dell, Macintosh), portable computers, mainframes, minicomputers, or other computing devices.

The input components can be complete, stand-alone personal computers offering a full range of power and features to run applications. The user component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe, or distributed across multiple servers (e.g., as in cloud computing applications) and offers data management, information sharing between clients, network administration and security. The application and any databases used can be on the same or different servers. Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

In certain embodiments, the present invention provides methods for obtaining a subject's risk profile for developing gastrointestinal neoplasm (e.g., colorectal cancer, gastric cancer, pancreatic cancer, liver cancer, cancers of the gall bladder and/or bile ducts (e.g., cholangiocarcinoma)). In some embodiments, such methods involve obtaining a stool, bile or blood sample from a subject (e.g., a human at risk for developing gastrointestinal cancer; a human undergoing a routine physical examination), detecting the presence, absence, or level (e.g., methylation frequency or score) of one or more markers specific for a gastrointestinal neoplasm in or associated with the stool, blood, plasma, bile or serum sample (e.g., specific for a gastrointestinal neoplasm) in the stool, blood, plasma, bile or serum sample, and generating a risk profile for developing gastrointestinal neoplasm (e.g., colorectal cancer, gastric cancer, pancreatic cancer, liver cancer, cancers of the gall bladder and/or bile ducts (e.g., cholangiocarcinoma)) based upon the detected level (score, frequency) or presence or absence of the indicators of gastrointestinal neoplasia. For example, in some embodiments, a generated risk profile will change depending upon specific markers and detected as present or absent or at defined threshold levels. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific exfoliated epithelial markers as determined with the methods of the present invention. In some embodiments, the presence and absence of specific markers as determined with the methods of the present invention are imputed into such an algorithm, and the risk profile is reported based upon a comparison of such input with established norms (e.g., established norm for pre-cancerous condition, established norm for various risk levels for developing gastrointestinal cancer, established norm for subjects diagnosed with various stages of gastrointestinal cancer). In some embodiments, the risk profile indicates a subject's risk for developing gastrointestinal cancer or a subject's risk for re-developing gastrointestinal cancer. In some embodiments, the risk profile indicates a subject to be, for example, a very low, a low, a moderate, a high, and a very high chance of developing or re-developing gastrointestinal cancer. In some embodiments, a health care provider (e.g., an oncologist) will use such a risk profile in determining a course of treatment or intervention (e.g., biopsy, wait and see, referral to an oncologist, referral to a surgeon, etc.).

The following examples are provided in order to demonstrate and further illustrate certain preferred embodiments and aspects of the present invention and are not to be construed as limiting the scope thereof.

### Example 1

### Identification of Novel Epigenetic Biomarkers in Colorectal Cancer, GLDC and PPP1R14A

### Materials:

A panel of twenty colon cancer cell lines was analysed in this project. The panel included eleven microsatellite stable (MSS; ALA, Colo320, EB, FRI, HT29, IS1, IS2, IS3, LS1034, SW480, V9P) and nine microsatellite unstable (MSI; Co115, HCT15, HCT116, LoVo, LS174T, RKO, SW48, TC7, TC71) cell lines, thereby representing both of the phenotypical subgroups of colorectal cancer. Forty-seven primary colorectal carcinoma samples, including 27 MSS and 20 MSI tumours, were subjected to DNA promoter methylation analysis in the present study. Twenty-four of the samples derived from a series which was collected at seven hospitals in the South-Eastern part of Norway from 1987-1989. The remaining 23 samples were collected at Aker University Hospital from 2005-2007. Also included in the present project were 49 normal colorectal mucosa samples derived from deceased colorectal cancer-free individuals.

### Methods:

### Genome-Wide Gene Expression Analysis

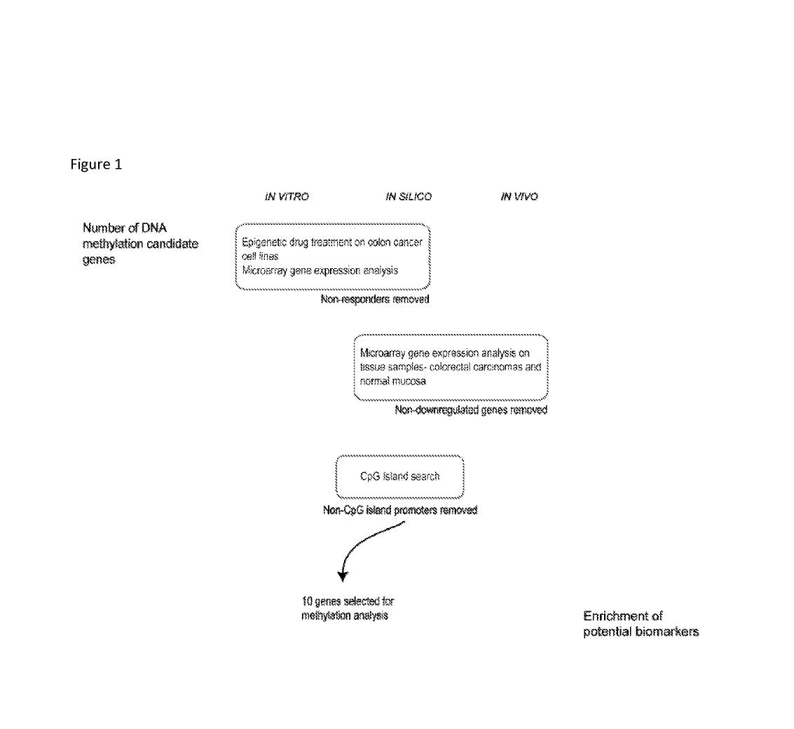

The AB1700 microarray platform was utilised to analyse the gene expression of colon cancer cell lines before and after treatment with AZA and TSA, identifying novel gene targets epigenetically inactivated in colorectal tumorigenesis. Six cell lines were analysed, including three MSI (SW48, RKO, HCT15) and three MSS (SW480, LS1034, HT29) cell lines. Only genes up-regulated four or more times after treatment in at least five of the six cell lines analysed were chosen for further investigation. In order to increase the likelihood of selecting true epigenetic targets, the gene expression of the same genes were analysed in primary colorectal carcinomas and normal tissue samples, using the same microarray platform. Only those genes that responded in cell lines and simultaneously were down-regulated in the carcinomas as compared to normal tissue were chosen for further analysis. The selection process for the discovery of new, hypermethylated genes is summarised in FIG. 1.

### Methylation-Specific Experimental Analyses:

Because loss of gene expression often is associated with aberrant methylation of promoter CpG islands, suitable target genes for DNA methylation analysis should contain a CpG island in their promoter region. Therefore, CpG Island Searcher was applied to analyse the candidate genes for the presence of one or more islands. Bisulfite treatment, qualitative and quantitative methylation-specific polymerase chain reaction as well as direct bisulfite sequencing was performed on the most promising genes from the genome wide gene expression studies.

### Statistics:

All 2×2 contingency tables were analysed using a two-sided Fisher's exact test, whereas a two-sided Pearson Chi-square test was used on 2×3 and 2×4 contingency tables. P values less than or equal to 0.05 (5%) were considered significant. Binary regression analyses were used to examine possible association between DNA methylation and patient age. Receiver Operating Characteristics (ROC) curves for individual genes were created using PMR values and tissue type (carcinoma and normal) as input. All calculations are derived from two-tailed statistical tests using the SPSS 16.0 software.

### Results:

### Qualitative and Quantitative Methylation Analyses of Candidate Genes in Cell Lines and Tissue Samples.

Among 20 colon cancer cell lines, BNIP3, CBS, DDX43, GLDC, IQCG, PEG10, PPP1R14A, RASSF4, RBP7 and WDR21B were methylated in 70%, 74%, 89%, 75%, 0%, 90%, 95%, 5%, 45% and 100%, respectively. The six most frequently hypermethylated genes were subjected to methylation analysis in a pilot of primary colorectal carcinomas and normal mucosa samples. BNIP3, GLDC, and PPP1R14A were methylated in 8, 14 and 11 of the carcinomas and in 1, 0 and 0 of the normal samples, respectively.

GLDC and PPP1R14A were selected for further investigation by real-time quantitative MSP in a larger series of malignant and normal colorectal tissue samples.

### Quantitative Methylation Profiles of GLDC and PPP1R14A.

In the larger validation study, promoter hypermethylation for GLDC and PPP1R14A were found in 60% and 57% of the primary colorectal tumours. Samples were scored as positive for methylation if the PMR value was >3.5 for PPP1R14A and 2.5 for GLDC. With these cut-off values, none of the normal mucosa samples for either gene were scored as methylated, resulting in 100% specificity for both assays.

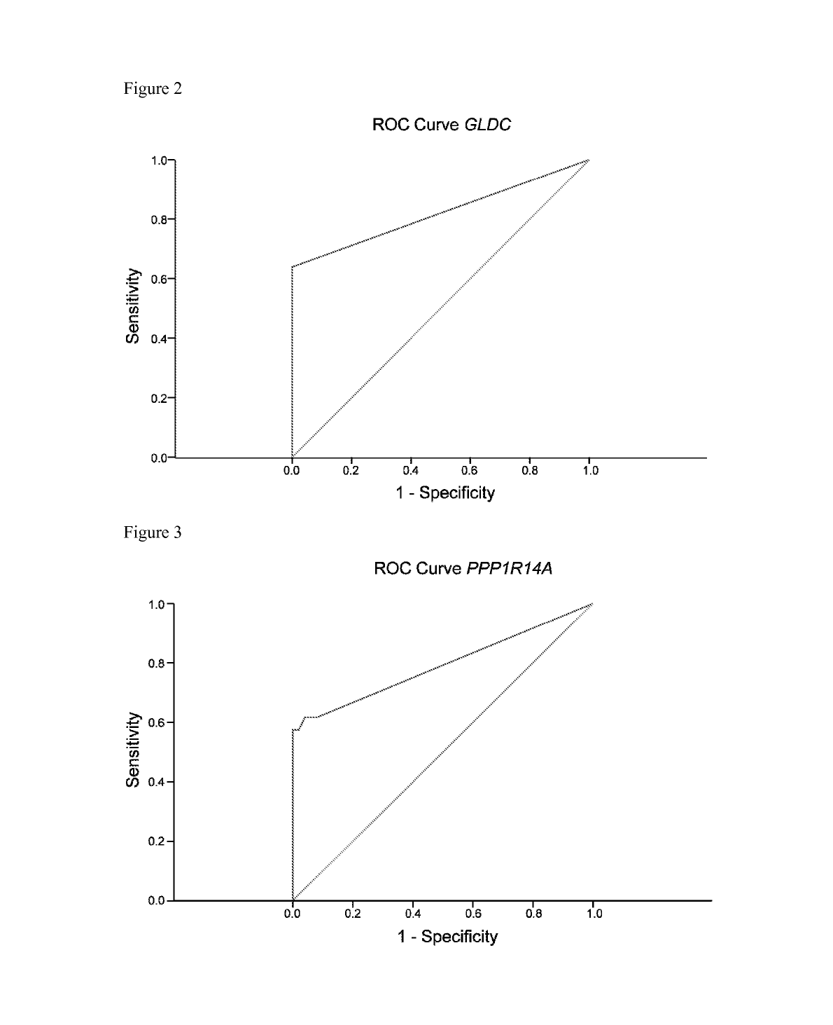

ROC curve analysis was applied to provide a statistical method to assess the diagnostic accuracy of the genes as biomarkers. GLDC had a sensitivity of 64% and a specificity of 100%, with an area under the curve (AUC) of 0.819 (P=7·10⁻⁸). PPP1R14A had a sensitivity of 57.5% and a specificity of 100%, with an AUC of 0.792 (P=8.59·10⁻⁷). The ROC curves are visualised in FIG. 2 and FIG. 3.

### Concordance of Conventional MSP and Quantitative Real-Time MSP

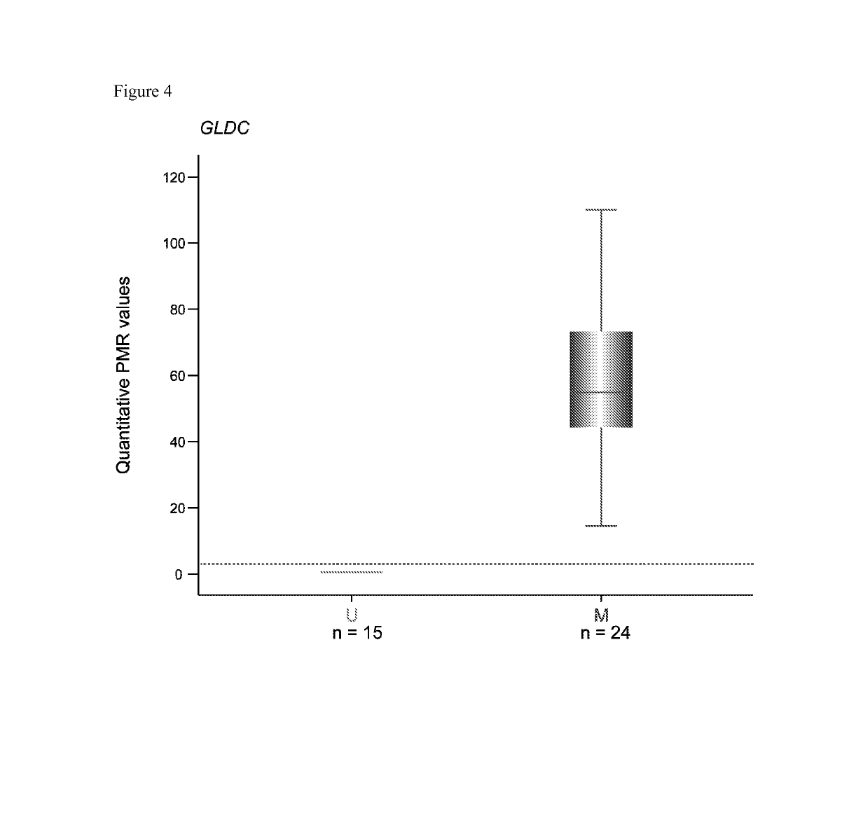

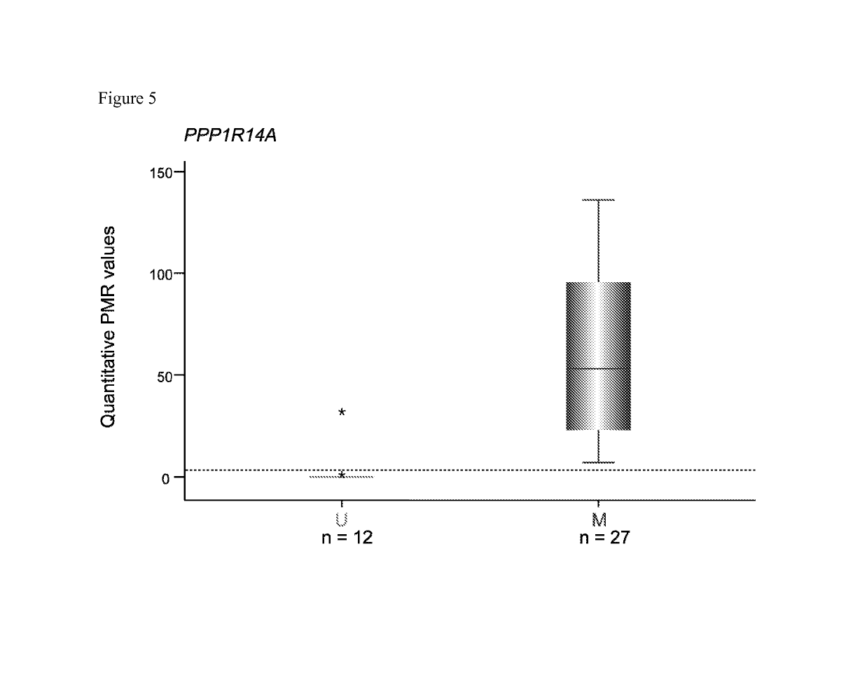

The results of qMSP analyses were compared with those obtained by conventional MSP in colon cancer cell lines (n=20), primary tumours (n=11) and normal tissue samples (n=8). While conventional MSP scores samples as methylated, partially methylated or unmethylated for cell lines, and methylated or unmethylated for tissue samples, qMSP data gives a quantitative measurement of DNA methylation levels ranging from 0 to 100 (FIG. 4). The cut-off values of 2.5 for GLDC and 3.5 for PPP1R14A resulted in good concordance between data obtained from qMSP and conventional MSP analyses. For GLDC, 39/39 (100%) of the samples were concordant (P=0.000). PPP1R14A, however, had one sample which was scored as unmethylated from qualitative, gel-based MSP and as methylated from the quantitative real-time MSP analysis. Consequently, the methylation status was in agreement for 38/39 (97%) of the samples (P=2·10⁻⁹). The results are illustrated in FIG. 4 and FIG. 5 and summarised in table 1 and table 2.

TABLE 1
Concordance of classification of the GLDC status by the two methods.

| | Quantitative real-time | | |
|---|---|---|---|
| | MSP with cut-off = 2.5 | | |
| Conventional MSP | Unmethylated | Methylated | Total |
| Unmethylated | 15 | 0 | 15 |
| Methylated | 0 | 24 | 24 |
| Total | 15 | 24 | 39 |

TABLE 2
Concordance of classification of the PPP1R14A status by the two
methods.

| | Quantitative real-time | | |
|---|---|---|---|
| | MSP with cut-off = 3.5 | | |
| Conventional MSP | Unmethylated | Methylated | Total |
| Unmethylated | 11 | 1 | 12 |
| Methylated | 0 | 27 | 27 |
| Total | 11 | 28 | 39 |

### Bisulfite Sequencing Confirms the Promoter Methylation Status of GLDC and PPP1R14A

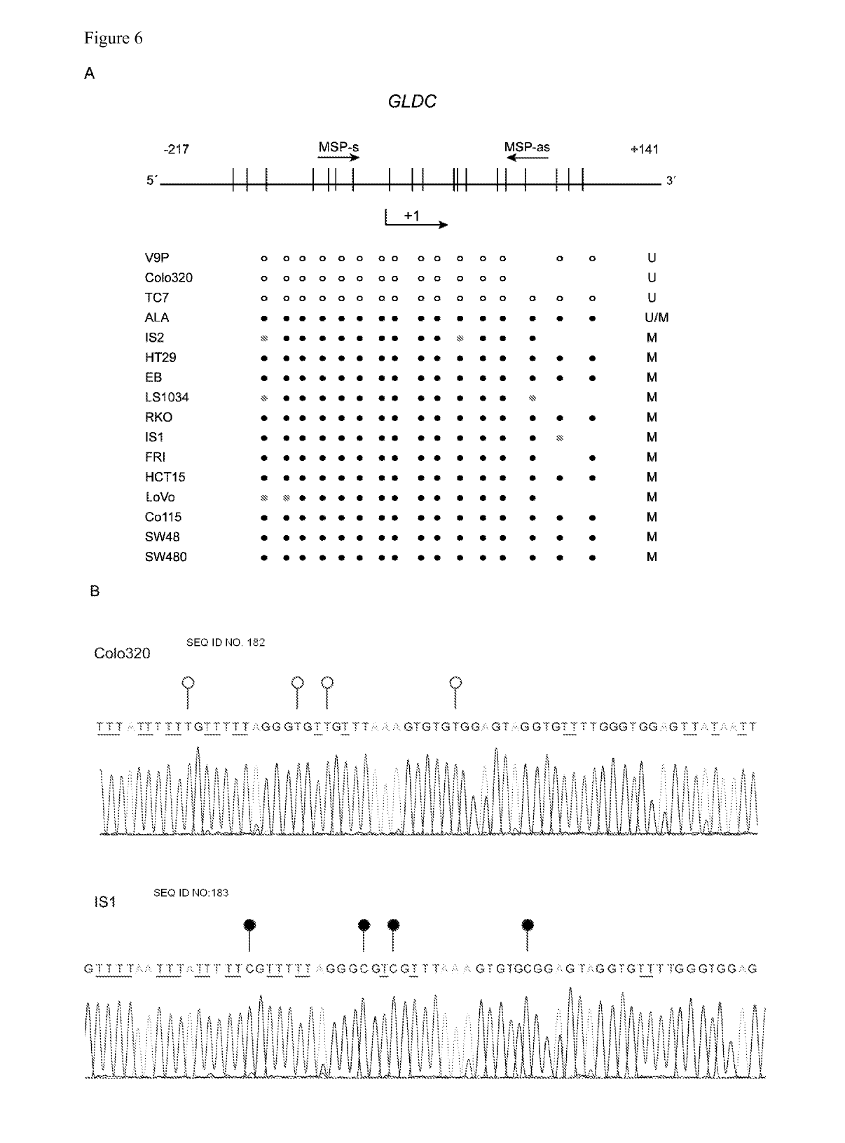

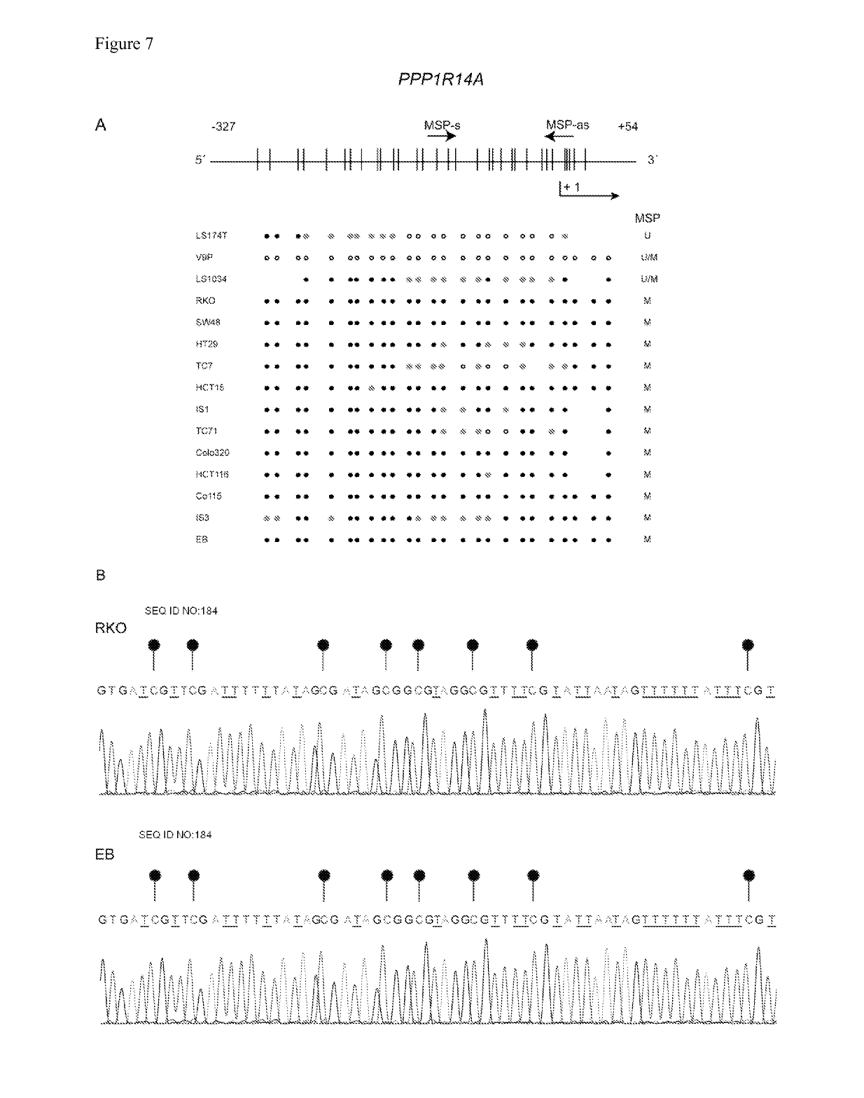

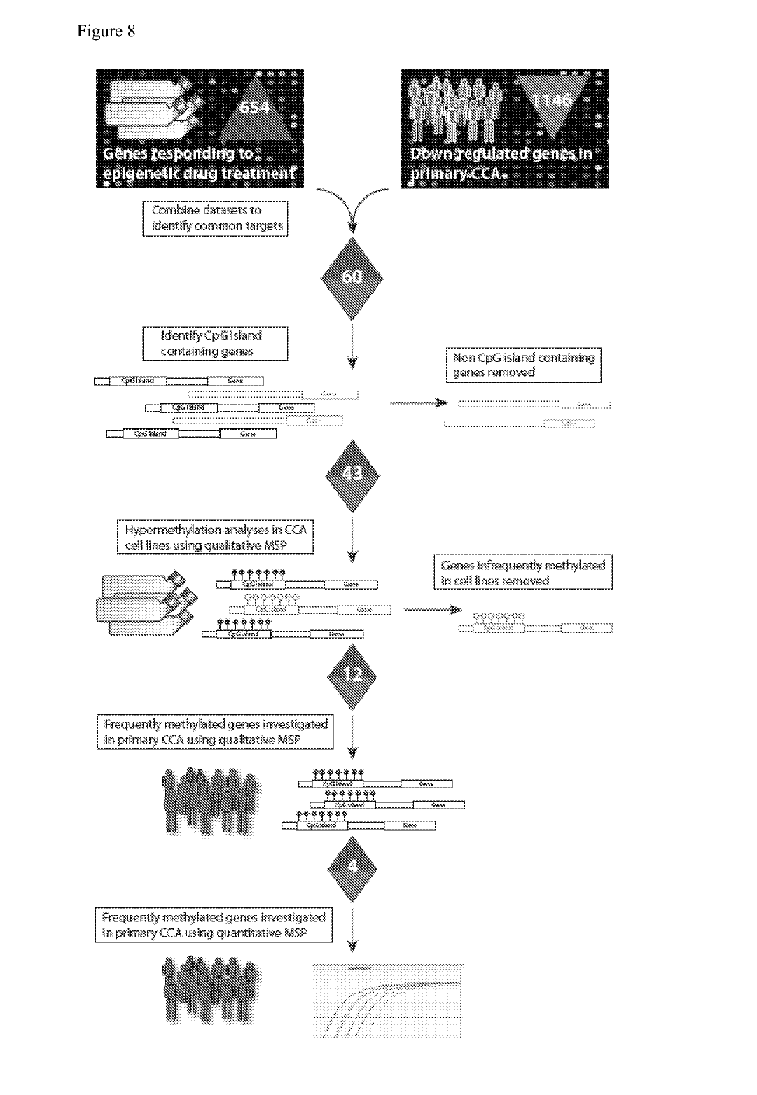

Bisulfite sequencing of GLDC and PPP1R14A in colon cancer cell lines showed that all non-CpG cytosines were fully converted to thymine. These results, along with detailed sequencing results and MSP status are shown in FIG. 6 and FIG. 7. In general, the majority of the cell lines that were scored as fully methylated by MSP, were also fully methylated from the bisulfite sequencing analyses.

Association of DNA Tumour Methylation with Genetic and Clinico-Pathological Features

DNA methylation status for GLDC and PPP1R14A were compared with genetic and clinico-pathological features of the tumours. Promoter methylation was independent of tumour stage, age and gender of the patients. PPP1R14A was significantly more methylated in tumours with microsatellite instability and thus in tumours located on the right side of the colon.

### Example 2

### Identification of Novel Epigenetic Biomarkers in Gastrointestinal Cancer (Cholangiocarcinomas), CDO1, DCLK1, ZNF33, and ZSCAN

Cholangiocarcinoma (CCA) is notoriously difficult to diagnose and displays a high mortality due to late clinical presentation. CpG island promoter hypermethylation is associated with cancer development. We aimed to identify novel epigenetic biomarkers with a potential to improve the diagnostic accuracy of CCA. Microarray analyses performed in CCA cell lines were compared with previously published expression profiles in tumors compared to non-malignant controls. Common candidate genes were interrogated for their promoter methylation status in cancer cell lines from the gastrointestinal tract, using a qualitative methylation specific polymerase chain reaction (MSP). Frequently methylated genes were subjected to quantitative methylation specific polymerase chain reaction (qMSP) in two CCA sample series, including fresh frozen (n=34) and formalin-fixed paraffin embedded tissues (n=59). From microarray analyses, 43 genes with a CpG island in their promoter region responded to epigenetic treatment and were simultaneously down-regulated in cholangiocarcinomas compared with non-malignant controls. We identified twelve genes as frequently hypermethylated in CCA cell lines. Of these, CDO1, DCLK1, SFRP1, and ZSCAN18 displayed high methylation frequencies also in tumors. Non-malignant samples were unmethylated for the same genes. The combined sensitivity of at least one positive among the four markers was 100% for fresh frozen tumors and 81% for archival tumors, and the specificity was 100% for both series. The resulting area under the receiver operating characteristics curve was 0.996 and 0.904, respectively. In conclusion, the novel epigenetic biomarker panel showed high sensitivity and specificity for CCA.

This example describes applications of an epigenome-wide approach (25) to identify a list of DNA methylation candidate genes in CCA. Potential target genes have been subjected to qualitative and quantitative promoter methylation analyses in cancer cell lines and patient material and three novel potential biomarkers for CCA have been identified.

### Materials and Methods

### Experimental Approach

The step-wise experimental approach used in the present study is illustrated in FIG. 8. Briefly, genes responding to epigenetic drug treatment in cancer cell lines were compared with a list of genes down-regulated in CCA samples versus non-malignant tissue. Responding and simultaneously down-regulated genes harboring a CpG island in the promoter region were subjected to qualitative methylation analysis in cancer cell lines. The most frequently methylated genes were further subjected to qualitative and subsequently quantitative methylation analyses in patient material.

### Cancer Cell Lines

Twenty-four cancer cell lines were analyzed, including bile duct (n=6; EGI-1, HuCCT1, KMBC, KMCH-1, SK-ChA-1, and TFK-1), colon (n=6; HCT15, HT29, LS1034, RKO, SW48, and SW480), pancreas (n=6; AsPc-1, BxBc-3, CFPAC-1, HPAFII, PaCa-2, and Panc-1), liver (n=4; HB8065, JHH-1, JHH-4, and JHH-5), and gallbladder (n=2; Mz-ChA-1 and Mz-ChA-2) cancers. The cell lines were cultured according to the manufacturer's guidelines, and the conditions are summarized in Table 5. All cell lines were harvested before reaching confluence.

Cell line authentication was performed using the AmpFLSTR Identifiler PCR Amplification Kit (Applied Biosystems, CA, USA) according to manufacturer's protocol. Samples were run on an AB Prism 3730 and analyzed in GeneMapper (Applied Biosystems). For commercially available cancer cell lines the genotypes were compared with previously published data. Results for non-commercial cell lines are listed in Table 6.

The six cholangiocarcinoma cell lines were subjected to treatment with a combination of the demethylating drug 5-aza-2′ deoxycytidine (1 mM for 72 hours; Sigma-Aldrich Company Ltd., Dorset, UK) and the histone deacetylase trichostatin A (1 mM added the last 12 hours; Sigma-Aldrich, Dorset, UK). Untreated controls were cultured in parallel.

### Patient Samples

### Fresh Frozen Material

Thirteen bile duct carcinomas were derived from patients undergoing surgery at Oslo University Hospital, Rikshospitalet, and Imperial College, London, UK. Samples were snap-frozen immediately after surgery and stored at −80° C. Carcinomas were embedded in Tissue-Tek (Sakura Finetek, CA, USA), according to the manufacturer's protocol, and subsequently subjected to cryo-sectioning and haematoxylin and eosin staining before they were evaluated by an expert pathologist. All carcinoma samples included (n=13) displayed >5% tumor cells. A cohort of 21 samples from non-malignant liver diseases, including autoimmune hepatitis (n=2), alcohol related liver disease (n=5), cryptogenic cirrhosis (n=1), hemochromatosis (n=1), primary biliary cirrhosis (n=3), and primary sclerosing cholangitis (n=9) were used as non-malignant controls. In addition, tissue from dissected bile ducts from six cancer free patients with primary sclerosing cholangitis was included as a separate sample set. The non-malignant biopsies were obtained from the peripheral liver region of patients undergoing surgery at Oslo University Hospital, Rikshospitalet.

### Archival Material

The archival sample series comprised 26 CCA- and 33 non-malignant formalin-fixed, paraffin-embedded samples obtained from the Department of pathology, Oslo University Hospital. The tissue specimens were routinely stained with haematoxylin and erythrosine. All carcinoma samples included (n=26) displayed >5% tumor cells. Tissue from non-malignant liver disease patients, including autoimmune hepatitis (n=4), alcohol related liver disease (n=1), primary biliary cirrhosis (n=4), and primary sclerosing cholangitis (n=21), as well as disease free patients (n=3) were included as non-malignant tissue controls. Liver biopsies were obtained from extrahepatic bile duct, hilum, and peripheral liver region.

### Gene Expression Microarray Analyses of Cancer Cell Lines

RNA from six CCA cell lines and their epigenetic drug treated counterparts were subjected to gene expression microarray analyses (Applied Biosystems). Genes up-regulated at least two-fold after 5-aza-2′ deoxycytidine and trichostatin A treatment in a minimum of four of the analyzed cancer cell lines, were considered to be potential targets for DNA methylation.

Microarray Gene Expression Data Sets from Cholangiocarcinoma Patients and Healthy Controls

In order to increase the likelihood of identifying putative hypermethylated genes in CCA, the gene list generated from the microarray approach in CCA cell lines was compared with gene lists generated from published and available gene expression microarray data comparing CCAs with non-malignant controls (26;27). Only responding genes from the cell line approach that were simultaneously down-regulated in CCA compared to non-malignant controls, were considered methylation candidates and subjected to further analyses.

### DNA Promoter Methylation Analyses

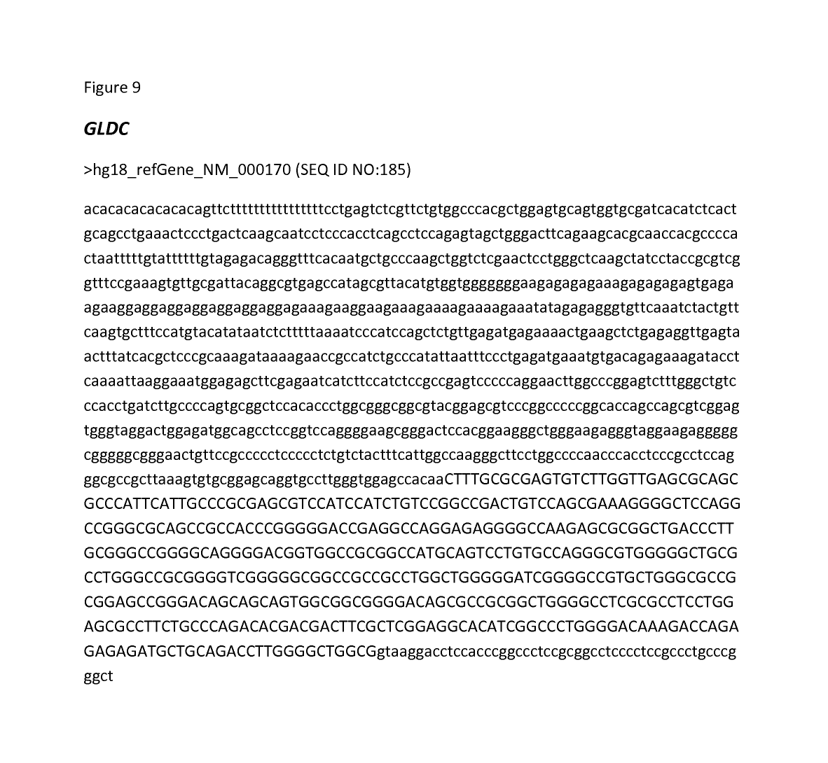

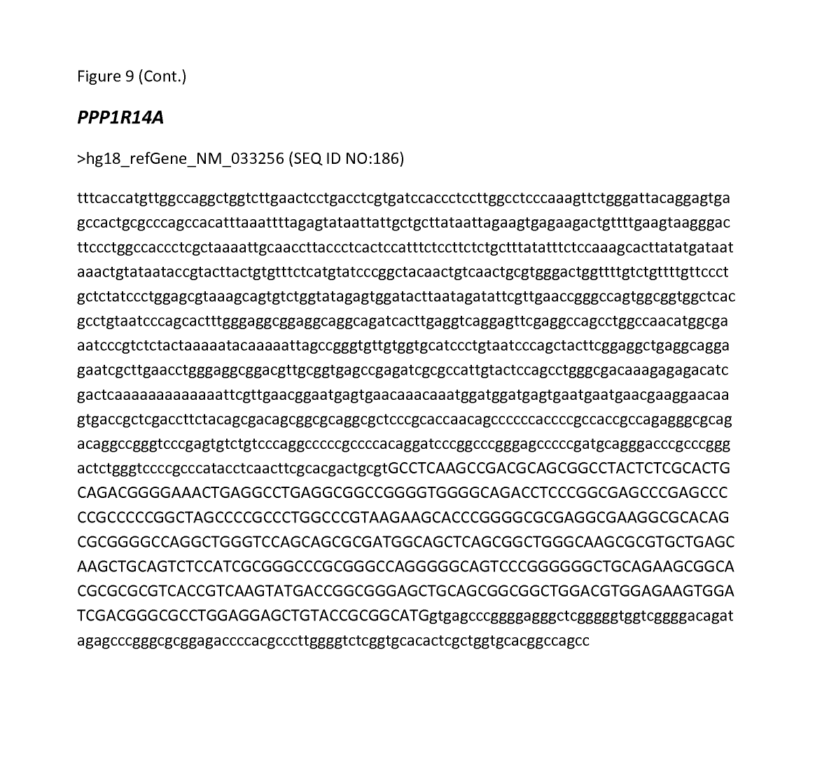

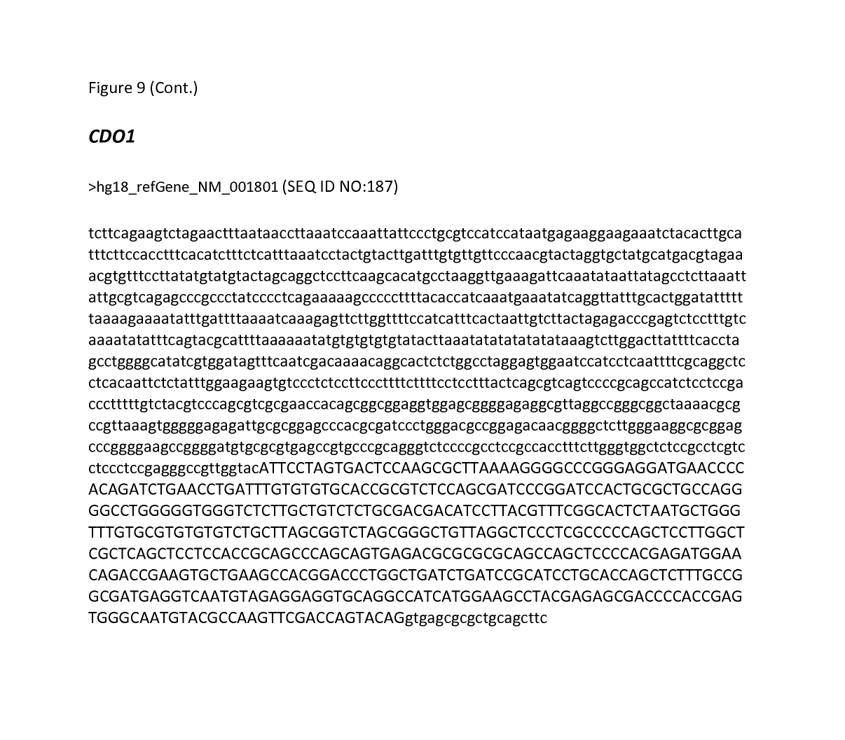

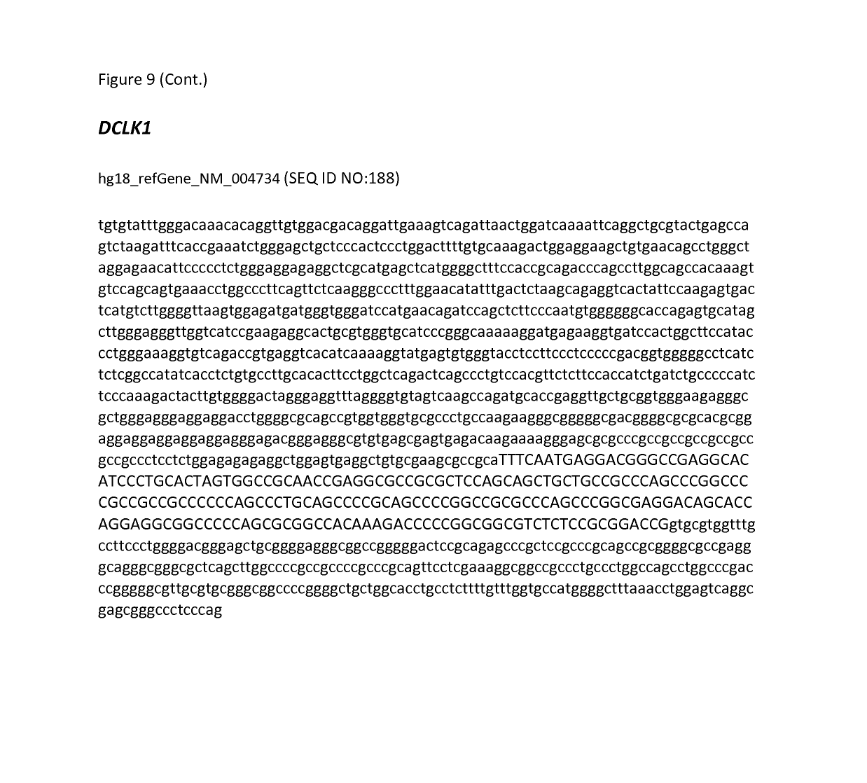

Candidate genes were interrogated for CpG island in the promoter region. Bisulfite treatment was performed prior to DNA methylation analyses. Qualitative methylation-specific polymerase chain reaction (MSP) was performed in cancer cell lines. Genes methylated in a minimum of five CCA cell lines were further subjected to MSP in fresh frozen tissue samples (n=34). The best performing genes (CDO1, DCLK1, and ZSCAN18) were subjected to direct promoter bisulfite sequencing. Primer sequences and -location, amplicon length, MgCl₂ concentration, and annealing temperatures are listed in Table 1. The methylation status of the three abovementioned genes in addition to the previously reported SFRP1 was finally assessed in both fresh frozen- and archival patient material using quantitative methylation-specific polymerase chain reaction (qMSP). Sequences are listed in Table 2. Table 3 provides detailed information about the genes. FIG. 9 shows sequences of the genes in Table 3.

### Nucleic Acid Isolation

For cancer cell lines, DNA was isolated using a standard phenol/chloroform procedure and total RNA was isolated using Trizol (Invitrogen, Carlsbad, Calif.) according to manufacturer's instructions. RNA quality was assessed using a 2100 bioanalyzer (Agilent Technologies, Palo Alto, Calif.). For fresh frozen samples, DNA was extracted from approximately 25 mg of tissue using AllPrep DNA/RNA kit (Qiagen Inc, Valencia, Calif.). For archival material DNA was isolated from five sections of paraffin embedded tissue á 20 μm, using QIAamp DNA kit (Qiagen). Nucleic acid concentration was determined using the ND-1000 Nanodrop (NanoDrop Technologies, Wilmington, Del.).

### Description of the Gene Expression Microarray Analysis

Standard one-round amplification was performed using the NanoAmp RT-IVT Labeling Kit (Applied Biosystems) according to the manufacturers' protocol. Briefly, cDNA was synthesized from one μg total RNA in an oligo dT primed reaction. Labeled complementary RNA (cRNA) was obtained from double-stranded cDNA in the presence of digoxigenin (DIG)-UTP in an in vitro labeling reaction. Samples were hybridized to gene expression microarrays (Human Genome Survey Microarray V2.0, Applied Biosystems) containing 32,878 oligonucleotide probes representing 29,098 individual genes. Chemoluminescense was measured using the AB1700 Chemoluminescense Analyzer (Applied Biosystems) after incubating the array with alkalic phosphatase-linked digoxygenin antibody. Probe signals were post-processed and quantile normalized using the R-script (R 2.5.0) “ABarray” file 1.2.0 and Bioconductor (www.bioconductor.org) and further analyzed in Excel (Microsoft office, version 2007). Array elements with a signal-to-noise ratio <3, and/or a flag value >8191 were discarded from further analysis.

### CpG Island Search

The candidate genes for DNA methylation were interrogated for the presence of a CpG island in the promoter region using the CpG island searcher algorithm (48). Default criteria were applied for CpG island detection (49).

### Bisulfite Treatment of DNA

Bisulfite treatment of DNA results in the conversion of unmethylated but not methylated cytosines to uracil (50;51). DNA (1.3 μg) was bisulfite treated using the EpiTect bisulfite kit (Qiagen) according to manufacturers' protocol. Desulfonation and washing steps were performed using a QIAcube (Qiagen) and the bisulfite treated DNA was eluted in 40 μl elution buffer.

### Qualitative Methylation-Specific Polymerase Chain Reaction (MSP)

MSP primers were designed in close proximity of the transcription start site, according to the Human Genome browser (genome.ucsc.edu), using the Methyl Primer Express Software v1.0 (Applied Biosystems). Primers were purchased from MedProbe (MedProbe, Oslo, Norway). Two pairs of primers were used to amplify the loci of interest, one specific for methylated- and one specific for unmethylated template. The MSP mixture contained one unit HotStarTaq DNA polymerase (Qiagen), 1×PCR buffer (Tris-Cl, KCl(NH₄)₂SO₄, 15 mM MgCl₂; pH 8.7; Qiagen), deoxynucleotide triphosphates (each at 2.5 mM), primers (10 μM each) and 24 ng of template DNA in a total volume of 25 μl. All MSP reactions were optimized with respect to MgCl₂ concentration, annealing temperature and elongation time (Table 2). Thermal cycling (Tetrad 2, Bio-Rad, CA, USA) was performed at 95 C.° for 15 minutes and 35 subsequent cycles (30 seconds at 95° C., 30 seconds at variable annealing temperatures (Table 2), and 30-60 seconds elongation at 72° C.) with a final extension of seven minutes at 72° C. Human placental DNA (Sigma Chemical Co, St Louis, Mo.) treated in vitro with SssI methyltransferase (New England Biolabs Inc., Beverly, Mass.) was used as positive control for the methylated reaction and DNA from normal lymphocytes was used as a positive control for the unmethylated reaction. Water, replacing the template was used as a negative control in both reactions. MSP products were mixed with five μl gel loading buffer (1×TAE buffer, 20% Ficoll; Sigma Aldrich, and 0.1% xylen cyanol; Sigma Aldrich), and loaded onto a 2% agarose gel (BioRad, Hercules, Calif., USA) in 1×TAE and ethidium bromide (Sigma Aldrich). Electrophoresis was performed at 200V for 25 minutes before MSP products were visualized by UV irradiation using a Gel Doc XR+(BioRad). All results were verified by a second independent round of MSP and scored independently by two authors. In cases of discrepancies, a third round of analysis was performed. Scoring was performed using the methylated band intensities of positive controls as reference. In tissue samples, the methylated band intensities were scored on a scale from 0-5. Tumor samples were only considered hypermethylated when band intensities were equal to or stronger than three. For non-malignant samples, methylated band intensities equal to or stronger than three were scored as hypermethylated, while methylated band intensities equal to or below two were determined weakly methylated. Only non-malignant samples not displaying MSP fragments for the methylated reaction were scored as unmethylated.

### Direct DNA Bisulfite Sequencing

Fourteen cancer cell lines (colon, n=6; cholangiocarcinoma, n=6 and gallbladder carcinoma, n=2) were subjected to bisulfite sequencing. DNA bisulfite primers were designed using the Methyl Primer Express Software v1.0 (Applied Biosystems) to cover the area amplified by the MSP. The experimental procedure has been published previously (52). Briefly, fragments were amplified for 35 cycles using HotStarTaq (Qiagen) and purified using Exo SAP-IT, according to the protocol of the manufacturer (GE Healthcare, USB Corporation, Ohio, USA). Sequencing was performed using the dGTP BigDye Terminator Cycle Sequencing Ready Reaction kit on the AB Prism 3730 (Applied Biosystems). Bisulfite-treated completely methylated DNA (CpGenome Universal Methylated DNA, Millipore, Mass., USA) and DNA from normal lymphocytes served as positive and negative controls, respectively. In accordance with a previous report (53), the amount of methylcytosine of each CpG site was calculated by the peak height ratio of the cytosine signal versus the sum of cytosine and thymine signal. Methylation of individual CpG sites was determined after the following criteria: 0-0.2 was unmethylated, 0.21-0.8 was partially methylated and 0.81-1.0 was scored as hypermethylated.

Quantitative Methylation-Specific Polymerase Chain Reaction (qMSP)

Primers and probes were designed using Primer Express v3.0 and purchased from Medprobe and Applied Biosystems, respectively. All qMSP reactions were carried out in triplicates in 384-well plates. The total reaction volume was 20 μl, and included 0.9 μM of each primer, 0.2 μM probes (labelled with 6-FAM and a non-fluorescent quencher), 30 ng bisulfite treated template and 1× TaqMan Universal PCR master mix NoAmpErase UNG (Applied Biosystems). Amplification was performed at 95° C. for 15 minutes before 45 cycles of 15 seconds at 95° C. and 1 minute elongation at 60° C., using the TaqMan 7900HF (Applied Biosystems). Bisulfite-converted completely methylated DNA (Millipore) served as a positive control and was also used to generate a standard curve by 1:5 serial dilutions (32.5-0.052 ng). The ALU-C4 gene (54) was used for normalization. In addition, bisulfite treated and untreated DNA from normal lymphocytes, and water blanks were used as negative controls.

All samples were censored after cycle 35 (in accordance with Applied Biosystems protocol) and the median quantity value was used for further processing. Briefly, percent methylated reference (PMR) was calculated by dividing the GENE:ALU ratio in the sample by the GENE:ALU ratio of the positive control (completely methylated DNA) and multiplying by 100. To ensure high specificity, individual fixed thresholds were established for each assay (one for fresh frozen material and one for archival material), using the integer above the highest PMR value across the normal samples. Samples with higher PMR values than the thresholds were scored as methylation positive. For CDO1, DCLK1, SFRP1, and ZSCAN18 the thresholds for the fresh frozen sample series were 1, 2, 1, and 1, respectively. For the archival sample series, thresholds were set at 2, 2, 5, and 3.

### Statistical Analyses

For statistical analyses, PASW 18.0 (SPSS, Chicago, Ill., USA) was used. Pearson's chi-square and Fishers exact tests were used for categorical variables. Student T-test and Mann-Whitney U test were used to investigate potential relationships between tumor DNA methylation and patient age. To evaluate the suitability of the methylated target genes to separate CCA from non-malignant controls, receiver operating characteristics curves were generated using the individual percentage methylated reference (PMR) values. Similarly, the combined performance of the candidate genes as a panel was evaluated using the sum of the PMR values. P-values were considered statistically significant at P<0.05.

TABLE 1
Primer sequences, fragment size and PCR conditions

| | | SEQ | | SEQ | Frg. | | | | El. | |
|---|---|---|---|---|---|---|---|---|---|---|
| Primer | Sense | ID | Antisense | ID | Size, | Fragment | An. | Mg | Time | Accesion |
| set | primer | NO. | primer | NO. | bp | location | Temp | Cl₂ | (sec) | number |
| ASRGL1 | GAGATAGGT | 1 | ACAACGA | 2 | 91 | +60 to | 57 | 1.5 | 30 | NM_00 |
| _MSP_M | GCGCGTTAG | | TTCTACG | | | +151 | | | | 108392 |
| | TC | | CCTACG | | | | | | | 6 |
| ASRGL1 | AGTGAGATA | 3 | ACAACAA | 4 | 94 | +57 to | 57 | 1.5 | 30 | |
| _MSP_U | GGTGTGTGT | | TTCTACA | | | +151 | | | | |
| | TAGTT | | CCTACAC | | | | | | | |
| | | | AC | | | | | | | |
| ATF3_M | AGCGAGTAC | 5 | AAAACGA | 6 | 174 | −221 to | 53 | 1.5 | 30 | NM_00 |
| SP_M | GTATATTTG | | AACCGAA | | | −47 | | | | 104061 |
| | GC | | AACG | | | | | | | 9 |
| ATF3_M | AGTAGTGAG | 7 | ACCAAAA | 8 | 180 | −224 to | 53 | 1.5 | 30 | |
| SP_U | TATGTATAT | | CAAAACC | | | −44 | | | | |
| | TTGGT | | AAAAACA | | | | | | | |
| BEX4_M | AGGGGTTGA | 9 | TCTAACG | 10 | 132 | −90 to | 55 | 1.5 | 30 | NM_00 |
| SP_M | TTCGAAAGT | | CCAAAAC | | | +42 | | | | 108042 |
| | TTC | | GAAACA | | | | | | | 5 |
| BEX4_M | GATAGGGGT | 11 | AACTCTA | 12 | 138 | −93 to | 55 | 1.5 | 30 | |
| SP_U | TGATTTGAA | | ACACCAA | | | +45 | | | | |
| | AGTTTT | | AACAAAA | | | | | | | |
| | | | CA | | | | | | | |
| CALCO | TACGTTTTT | 13 | CTTTTAC | 14 | 116 | −118 to | 55 | 1.5 | 30 | NM_02 |
| CO1_MS | TAGGATGTC | | CGCTACG | | | −2 | | | | 0898 |
| P_M | GC | | TACTCG | | | | | | | |
| CALCO | AATTATGTT | 15 | CCCTTTT | 16 | 121 | −121 to | 55 | 1.5 | 30 | |
| CO1_MS | TTTTAGGAT | | ACCACTA | | | 0 | | | | |
| P_U | GTTGT | | CATACTC | | | | | | | |
| | | | AA | | | | | | | |
| CDO1_ | TTGGGACGT | 17 | GACCCTC | 18 | 145 | −153 to | 53 | 1.5 | 30 | NM_00 |
| MSP_M | CGGAGATA | | GAAAAAA | | | −8 | | | | 1801 |
| | AC | | AAACGA | | | | | | | |
| CDO1_ | TTTTTGGGA | 19 | AACCCTC | 20 | 148 | −156 to | 53 | 1.5 | 30 | |
| MSP_U | TGTTGGAGA | | AAAAAAA | | | −8 | | | | |
| | TAAT | | AAACAAA | | | | | | | |
| | | | AC | | | | | | | |
| CDO1_B | TTTTTTTTGT | 21 | ACAAATC | 22 | 350 | −280 to | 49 | 1.7 | 30 | |
| S | TTAYGTTTT | | AAATTCA | | | +70 | | | | |
| | A | | AATCT | | | | | | | |
| CLU_MS | TTTTTTTTAT | 23 | AAAAAAT | 24 | 165 | −147 to | 52 | 2.4 | 30 | NM_00 |
| P_M | TGGAAGCGT | | ACCGCGA | | | +18 | | | | 1831 |
| | C | | AAAAC | | | | | | | |
| CLU_MS | GGTTTTTTT | 25 | CCAAAAA | 26 | 170 | −150 to | 52 | 2.4 | 30 | |
| P_U | TTATTGGAA | | ATACCAC | | | +20 | | | | |
| | GTGTT | | AAAAAAC | | | | | | | |
| | | | A | | | | | | | |
| CRISPL | TTCGTTTAT | 27 | ACTCAAC | 28 | 172 | −178 to | 52 | 1.5 | 30 | NM_03 |
| D2_MSP | TCGGCGTTC | | GTACCGC | | | −6 | | | | 1476 |
| _M | | | CTCTT | | | | | | | |
| CRISPL | TTTTTTGTTT | 29 | AAAACTC | 30 | 178 | −181 to | 52 | 1.5 | 30 | |
| D2_MSP | ATTTGGTGT | | AACATAC | | | −3 | | | | |
| _U | TT | | CACCTCT | | | | | | | |
| | | | T | | | | | | | |
| CSRP1_ | ACGTGTAAG | 31 | AACCCGA | 32 | 147 | −126 to | 55 | 1.7 | 30 | NM_00 |
| MSP_M | ACGTTTTTC | | CGATACT | | | +21 | | | | 4078 |
| | GC | | ACCCTC | | | | | | | |
| CSRP1_ | GTATGTGTA | 33 | AACCCAA | 34 | 149 | −128 to | 56 | 1.5 | 30 | |
| MSP_U | AGATGTTTT | | CAATACT | | | +21 | | | | |
| | TTGT | | ACCCTCC | | | | | | | |
| | | | T | | | | | | | |
| CTGF_M | TCGGAGCGT | 35 | CTATCGA | 36 | 122 | −34 to | 56 | 2.5 | 30 | NM_00 |
| SP_M | ATAAAAGTT | | CCGAAAC | | | +88 | | | | 1901 |
| | TC | | GACTAC | | | | | | | |
| CTGF_M | GTTTGGAGT | 37 | CTATCAA | 38 | 124 | −36 to | 56 | 2.5 | 30 | |
| SP_U | GTATAAAAG | | CCAAAAC | | | +88 | | | | |
| | TTTT | | AACTACC | | | | | | | |
| | | | A | | | | | | | |
| DCLK1_ | GCGTTTTGT | 39 | ACGCGCT | 40 | 108 | −127 to | 53 | 1.5 | 30 | NM_00 |
| MSP_M | TAAGAAGG | | CCCTTTT | | | −19 | | | | 4734 |
| | GC | | CTTAT | | | | | | | |
| DCLK1_ | GTGTTTTGT | 41 | ACACACT | 42 | 108 | −127 to | 53 | 1.5 | 30 | |
| MSP_U | TAAGAAGG | | CCCTTTT | | | −19 | | | | |
| | GT | | CTTAT | | | | | | | |
| DCLK1_ | AAGATTATT | 43 | AACCTCT | 44 | 271 | −247 to | 57 | 1.5 | 30 | |
| BS | TGTGGGGAT | | CTCTCCA | | | +24 | | | | |
| | TAGG | | AAAAAAA | | | | | | | |
| | | | A | | | | | | | |
| DUSP5_ | GAGTGAGTT | 45 | ATAAATA | 46 | 198 | −192 to | 52 | 1.5 | 30 | NM_00 |
| MSP_M | TTTTAGCGA | | CCGTCCG | | | +6 | | | | 4419 |
| | AGC | | TAACGC | | | | | | | |
| DUSP5_ | GAGTGAGTT | 47 | ATAAATA | 48 | 198 | −192 to | 52 | 1.5 | 30 | |
| MSP_U | TTTTAGTGA | | CCATCCA | | | +6 | | | | |
| | AGT | | TAACAC | | | | | | | |
| EGR2_ | TATATGGGT | 49 | TCGCCGA | 50 | 104 | −108 to | 52 | 2.0 | 30 | NM_00 |
| isoform | AGCGACGTT | | ACTATTA | | | −4 | | | | 113617 |
| “a”_MSP | AC | | ATCAATT | | | | | | | 7 |
| _M | | | A | | | | | | | |
| EGR2_ | TTATATATG | 51 | CCCTCAC | 52 | 110 | −111 to | 52 | 1.5 | 30 | |
| isoform | GGTAGTGAT | | CAAACTA | | | −1 | | | | |
| “a”_MSP | GTTAT | | TTAATCA | | | | | | | |
| _U | | | ATTA | | | | | | | |
| FAM3B_ | GGGGAACG | 53 | GCGACCA | 54 | 137 | −120 to | 53 | 1.5 | 30 | NM_05 |
| MSP_M | GGTTTATTT | | ATCGAAC | | | +17 | | | | 8186 |
| | TTC | | AAAT | | | | | | | |
| FAM3B_ | GGGGAATG | 55 | ACAACCA | 56 | 137 | −120 to | 53 | 1.5 | 30 | |
| MSP_U | GGTTTATTT | | ATCAAAC | | | +17 | | | | |
| | TTT | | AAAT | | | | | | | |
| FHL1_M | TCGTGTAGT | 57 | CTCCGCC | 58 | 165 | −160 to | 57 | 1.5 | 30 | NM_00 |
| SP_M | GGGTAGAGT | | GAACGAT | | | +5 | | | | 1449 |
| | TC | | AAAT | | | | | | | |
| FHLl_M | TTTTTGTGT | 59 | CCCCTCC | 60 | 171 | −163 to | 57 | 1.5 | 30 | |
| SP_U | AGTGGGTAG | | ACCAAAC | | | +8 | | | | |
| | AGTTT | | AATAAAT | | | | | | | |
| FKBP1B | GGTTCGTTA | 61 | CTAAAAT | 62 | 126 | −158 to | 55 | 2.0 | 30 | NM_05 |
| _MSP_M | ATAGTCGGG | | CGAAACC | | | −32 | | | | 4033 |
| | C | | TACGCG | | | | | | | |
| FKBP1B | TTAGGTTTG | 63 | ACTAAAA | 64 | 130 | −161 to | 52 | 1.5 | 30 | |
| _MSP_U | TTAATAGTT | | TCAAAAC | | | −31 | | | | |
| | GGGT | | CTACACA | | | | | | | |
| | | | AA | | | | | | | |
| GNG11_ | TCGGATGTG | 65 | CGCGAAA | 66 | 112 | −48 to | 56 | 1.5 | 30 | NM_00 |
| MSP_M | ATTTGGAAA | | AACGACT | | | +64 | | | | 4126 |
| | C | | AAACT | | | | | | | |
| GNG11_ | ATTTGGATG | 67 | CCCACAA | 68 | 116 | −50 to | 56 | 1.5 | 30 | |
| MSP_U | TGATTTGGA | | AAAACAA | | | +66 | | | | |
| | AAT | | CTAAACT | | | | | | | |
| GPR124_ | GGGTTTAGG | 69 | CCGCTCC | 70 | 119 | −124 to | 55 | 2.5 | 30 | NM_03 |
| MSP_M | TTTGGTCGC | | GTACCAT | | | −5 | | | | 2777 |
| | | | AAATAA | | | | | | | |
| GPR124_ | AGAGGGTTT | 71 | CCACCAC | 72 | 125 | −127 to | 55 | 1.5 | 30 | |
| MSP_U | AGGTTTGGT | | TCCATAC | | | −2 | | | | |
| | TGT | | CATAAAT | | | | | | | |
| | | | AA | | | | | | | |
| GREM1_ | AGTAGATAA | 73 | AAATACC | 74 | 172 | −198 to | 53 | 1.5 | 30 | NM_01 |
| MSP_M | AGAGGCGA | | GACGACA | | | −26 | | | | 3372 |
| | GGC | | AAACG | | | | | | | |
| GREM1_ | GGGAGTAG | 75 | AAATACC | 76 | 175 | −201 to | 53 | 1.5 | 30 | |
| MSP_U | ATAAAGAG | | AACAACA | | | −26 | | | | |
| | GTGAGGT | | AAACACA | | | | | | | |
| | | | A | | | | | | | |
| HABP4_ | CGTGACGTG | 77 | CTATCCG | 78 | 149 | −115 to | 58 | 1.5 | 30 | NM_01 |
| MSP_M | ATAGTAGTC | | ACCCCTA | | | +34 | | | | 4282 |
| | GGTC | | CCGAC | | | | | | | |
| HABP4_ | GTGTGATGT | 79 | CCTATCC | 80 | 151 | −116 to | 59 | 1.5 | 30 | |
| MSP_U | GATAGTAGT | | AACCCCT | | | +35 | | | | |
| | TGGTT | | ACCAAC | | | | | | | |
| ID3_MS | TTCGGAGGA | 81 | CGCTAAT | 82 | 173 | −32 to | 55 | 1.5 | 30 | NM_00 |
| P_M | GTTGTGGTT | | ACCGAAA | | | +141 | | | | 2167 |
| | C | | AAAAACG | | | | | | | |
| ID3_MS | GATTTTGGA | 83 | CACTAAT | 84 | 176 | −35 to | 55 | 1.5 | 30 | |
| P_U | GGAGTTGTG | | ACCAAAA | | | +141 | | | | |
| | GTTT | | AAAAACA | | | | | | | |
| | | | AAC | | | | | | | |
| INPP5A_ | TTAGCGGAT | 85 | TAACCGA | 86 | 113 | −20 to | 50 | 1.5 | 30 | NM_00 |
| MSP_M | TTAATGGTT | | AACTCCG | | | +93 | | | | 5539 |
| | GC | | ACCTC | | | | | | | |
| INPP5A_ | TTAGTGGAT | 87 | TAACCAA | 88 | 113 | −20 to | 50 | 1.5 | 30 | |
| MSP_U | TTAATGGTT | | AACTCCA | | | +93 | | | | |
| | GT | | ACCTC | | | | | | | |
| ITPR1_ | ATTTAGGGT | 89 | ACACTTT | 90 | 148 | −146 to | 55 | 2.5 | 30 | NM_00 |
| MSP_M | TTAGTTCGG | | AAAACGA | | | +2 | | | | 2222 |
| | GC | | CTCCGAA | | | | | | | |
| ITPR1_ | TTTATTTAG | 91 | ACTACAC | 92 | 154 | −149 to | 55 | 2.0 | 30 | |
| MSP_U | GGTTTAGTT | | TTTAAAA | | | +5 | | | | |
| | TGGGT | | CAACTCC | | | | | | | |
| | | | AAA | | | | | | | |
| LHX6_M | TGCGGTTGT | 93 | CCGAAAC | 94 | 100 | −69 to | 54 | 1.5 | 30 | NM_01 |
| SP_M | GGTTTTTTT | | GACGTTC | | | +31 | | | | 4368 |
| | C | | TCAT | | | | | | | |
| LHX6_M | TATTGTGGT | 95 | ACACCAA | 96 | 106 | −72 to | 54 | 1.5 | 30 | |
| SP_U | TGTGGTTTT | | AACAACA | | | +34 | | | | |
| | TTTT | | TTCTCAT | | | | | | | |
| LMCD1_ | GGTAGTCGG | 97 | CGCAACT | 98 | 165 | −176 to | 55 | 1.5 | 30 | NM_01 |
| MSP_M | CGTTTAGTT | | AAACCGC | | | −11 | | | | 4583 |
| | TC | | TTTAAT | | | | | | | |
| LMCD1_ | TAGGGTAGT | 99 | AAACACA | 100 | 171 | −179 to | 55 | 1.5 | 30 | |
| MSP_U | TGGTGTTTA | | ACTAAAC | | | −8 | | | | |
| | GTTTT | | CACTTTA | | | | | | | |
| | | | AT | | | | | | | |
| MLLT11 | TTTTTCGGG | 101 | AACCGAA | 102 | 110 | −118 to | 51 | 1.8 | 30 | NM_00 |
| _MSP_M | TTAGTTTTG | | CGAATTT | | | −8 | | | | 6818 |
| | C | | CGTAAT | | | | | | | |
| MLLT11 | GGGTTTTTT | 103 | CCAAACC | 104 | 116 | −121 to | 52 | 1.5 | 30 | |
| _MSP_U | GGGTTAGTT | | AAACAAA | | | −5 | | | | |
| | TTGT | | TTTCATA | | | | | | | |
| | | | AT | | | | | | | |
| MT1F_M | GTTTAGGGG | 105 | ACAACCG | 106 | 147 | −110 to | 55 | 1.5 | 30 | NM_00 |
| SP_M | ATTTTGCGT | | ACCGCTA | | | +37 | | | | 5949 |
| | TC | | CTTTAA | | | | | | | |
| MT1F_M | GTTTAGGGG | 107 | ACAACCA | 108 | 147 | −110 to | 55 | 1.5 | 30 | |
| SP_U | ATTTTGTGT | | ACCACTA | | | +37 | | | | |
| | TT | | CTTTAA | | | | | | | |
| MT1X_ | GGTTTACGG | 109 | AAAAACC | 110 | 129 | −136 to | 55 | 1.5 | 30 | NM_00 |
| MSP_M | GTTGTTGTA | | GACGACT | | | −7 | | | | 5952 |
| | TTC | | CTCTTT | | | | | | | |
| MT1X_ | GGGTTTATG | 111 | CAAAAAC | 112 | 131 | −137 to | 55 | 1.5 | 30 | |
| MSP_U | GGTTGTTGT | | CAACAAC | | | −6 | | | | |
| | ATTT | | TCTCTTT | | | | | | | |
| MT2A_ | GTGTGTAGA | 113 | AAAACCG | 114 | 132 | −108 to | 55 | 1.5 | 30 | NM_00 |
| MSP_M | GTCGGGTGC | | AAACGAA | | | −240 | | | | 5953 |
| | | | TACAAAA | | | | | | | |
| MT2A_ | GTGTGTAGA | 115 | AAAACCA | 116 | 132 | −108 to | 55 | 1.5 | 30 | |
| MSP_U | GTTGGGTGT | | AAACAAA | | | −240 | | | | |
| | | | TACAAAA | | | | | | | |
| NAP1L2 | GCGTAATTA | 117 | TACGTTA | 118 | 116 | +8 to | 56 | 1.5 | 30 | NM_02 |
| MSP_M | TATTGCGGT | | ACCGATC | | | +124 | | | | 1963 |
| | ATC | | CTACAA | | | | | | | |
| NAP1L2 | GTTGTGTAA | 119 | AACTACA | 120 | 122 | +5 to | 56 | 1.5 | 30 | |
| MSP_U | TTATATTGT | | TTAACCA | | | +127 | | | | |
| | GGTATT | | ATCCTAC | | | | | | | |
| | | | AA | | | | | | | |
| NR4A3_ | TTTTCGTAT | 121 | TCGACAC | 122 | 142 | −126 to | 52 | 1.5 | 30 | NM_17 |
| MSP_M | ACGCGGAAT | | GTCATTT | | | +16 | | | | 3198 |
| | C | | ATACCAC | | | | | | | |
| NR4A3_ | TTTTTTTTGT | 123 | CTCTCAA | 124 | 148 | −129 to | 52 | 1.5 | 30 | |
| MSP_U | ATATGTGGA | | CACATCA | | | +19 | | | | |
| | ATT | | TTTATAC | | | | | | | |
| | | | CAC | | | | | | | |
| PDE2A_ | ATTAGGCGA | 125 | CGACTCG | 126 | 161 | +10 to | 53 | 1.8 | 30 | NM_00 |
| MSP_M | AGTTGTCGC | | TCCGACT | | | +171 | | | | 114383 |
| | | | TAAAA | | | | | | | 9 |
| PDE2A_ | GGATTAGGT | 127 | AACAACT | 128 | 165 | +8 to | 53 | 1.8 | 30 | |
| MSP_U | GAAGTTGTT | | CATCCAA | | | +173 | | | | |
| | GT | | CTTAAAA | | | | | | | |
| REEP1_ | GGACGCGTT | 129 | AACCGCG | 130 | 149 | −162 to | 55 | 2.5 | 30 | NM_00 |
| MSP_M | CGTTTTTAG | | ACACGTT | | | −13 | | | | 116473 |
| | TC | | CTAAC | | | | | | | 2 |
| REEP1_ | GTAGGATGT | 131 | AACCACA | 132 | 152 | −165 to | 55 | 2.5 | 30 | |
| MSP_U | GTTTGTTTT | | ACACATT | | | −13 | | | | |
| | TAGTT | | CTAACAA | | | | | | | |
| | | | C | | | | | | | |
| RNASE4 | TAAATTTCG | 133 | TCGCGAA | 134 | 101 | −143 to | 53 | 2.5 | 30 | NM_00 |
| _MSP_M | GACGAGTTT | | ACAATTT | | | −42 | | | | 2937 |
| | TC | | ATATTTC | | | | | | | |
| RNASE4 | GTTTAAATT | 135 | CCATCAC | 136 | 107 | −146 to | 53 | 1.5 | 30 | |
| _MSP_U | TTGGATGAG | | AAAACAA | | | −39 | | | | |
| | TTTTT | | TTTATAT | | | | | | | |
| | | | TTC | | | | | | | |
| SFRP1_ | TAGTAAATC | 137 | TACGCGA | 138 | 141 | −138 to | 45 | 1.5 | 30 | NM_00 |
| MSP_M | GAATTCGTT | | AACTCCT | | | +3 | | | | 3012 |
| | CGC | | ACGAC | | | | | | | |
| SFRP1_ | TTTTAGTAA | 139 | TACACAA | 140 | 144 | −141 to | 45 | 1.5 | 30 | |
| MSP_U | ATTGAATTT | | AACTCCT | | | +3 | | | | |
| | GTTTGT | | ACAACCA | | | | | | | |
| | | | A | | | | | | | |
| SLC46A | GTTGAGTGG | 141 | CCCGACT | 142 | 151 | −152 to | 57 | 1.5 | 30 | NM_18 |
| 3_MSP_ | TTGTTCGGT | | CTCCTAC | | | −1 | | | | 1785 |
| M | C | | GATTAA | | | | | | | |
| SLC46A | GTGTTGAGT | 143 | TACCCAA | 144 | 155 | −154 to | 58 | 1.5 | 30 | |
| 3_MSP_ | GGTTGTTTG | | CTCTCCT | | | +1 | | | | |
| U | GTT | | ACAATTA | | | | | | | |
| | | | A | | | | | | | |
| SYT11_ | CGTTTTGGA | 145 | TTCCGAA | 146 | 158 | −222 to | 50 | 1.8 | 30 | NM_15 |
| MSP_M | ATTATAGCG | | TAATCCT | | | −64 | | | | 2280 |
| | C | | CGAAA | | | | | | | |
| SYT11_ | TTTTGTTTT | 147 | CTCTTCC | 148 | 164 | −225 to | 50 | 1.8 | 30 | |
| MSP_U | GGAATTATA | | AAATAAT | | | −61 | | | | |
| | GTGT | | CCTCAAA | | | | | | | |
| | | | A | | | | | | | |
| TCF4_M | GAATTTGTA | 149 | AAAAAAA | 150 | 258 | +322 to | 57 | 1.5 | 60 | NM_00 |
| SP_M* | ATTTCGTGC | | ACTCTCC | | | +580 | | | | 108396 |
| | GTTTC | | GTACACC | | | | | | | 2 |
| | | | G | | | | | | | |
| TCF4_M | TGAATTTGT | 151 | AAAAAAA | 152 | 259 | +321 to | 57 | 1.5 | 60 | |
| SP_U* | AATTTTGTG | | ACTCTCC | | | +580 | | | | |
| | TGTTTTG | | ATACACC | | | | | | | |
| | | | ACC | | | | | | | |
| TPM2_M | ATCGTCGGG | 153 | AACAAAA | 154 | 152 | −156 to | 61 | 1.5 | 30 | NM_00 |
| SP_M | GTTTTTTTA | | ACACGAC | | | −4 | | | | 114582 |
| | GTC | | CCGAC | | | | | | | 2 |
| TPM2_M | GTATTGTTG | 155 | AAACAAA | 156 | 155 | −158 to | 61 | 1.5 | 30 | |
| SP_U | GGGTTTTTT | | AACACAA | | | −3 | | | | |
| | TAGTT | | CCCAACC | | | | | | | |
| ZNF331_ | GGTAGGAC | 157 | ATACAAC | 158 | 143 | −120 to | 55 | 1.7 | 30 | NM_01 |
| isoform | GTTTTTAGG | | TCTACAC | | | +23 | | | | 8555 |
| “c”_MSP | GTC | | GACGCA | | | | | | | |
| _M | | | | | | | | | | |
| ZNF331_ | TAAGGTAGG | 159 | AACATAC | 160 | 143 | −120 to | 55 | 1.5 | 30 | |
| isoform | ATGTTTTTA | | AACTCTA | | | +23 | | | | |
| “c”_MSP | GGGTT | | CACAACA | | | | | | | |
| _U | | | CA | | | | | | | |
| ZSCAN1 | GTTTAAAAT | 161 | AATACCG | 162 | 131 | −52 to | 55 | 1.8 | 30 | NM_02 |
| 8_isoform | GACGTAGGC | | CGAAACT | | | +79 | | | | 3926 |
| “b”_MSP | GTC | | ATACCG | | | | | | | |
| _M | | | | | | | | | | |
| ZSCAN1 | GGTGTTTAA | 163 | ACAATAC | 164 | 131 | −55 to | 55 | 1.5 | 30 | |
| 8_isoform | AATGATGTA | | CACAAAA | | | +79 | | | | |
| “b”_MSP | GGTGTT | | CTATACC | | | | | | | |
| _U | | | AC | | | | | | | |
| ZSCAN1 | TTTTGGTTG | 165 | ACCCACC | 166 | 302 | −106 to | 59 | 1.5 | 30 | |
| 8_BS | TTAGGGGTT | | TACTACR | | | +196 | | | | |
| | TATT | | CAACTAC | | | | | | | |

From hg18 to hg19, the transcription start point of DCLK1 NM_004734 was moved 50 bp upstream. The MSP primers were originally designed to be located −177 to −69 relative to the transcription start site.
*Primer sequences and amplification conditions were obtained from Kim and colleagues (46).

TABLE 2
Assays used for quantitative methylation-
specific polymerase chain reaction (qMSP)

| | | SEQ | | SEQ | | SEQ | Frg. |
|---|---|---|---|---|---|---|---|
| | | ID | Antisense | ID | | ID | Size |
| Assay | Sense primer | NO. | primer | NO. | Probe | NO. | (bp) |
| ALU | GGTTAGGTAT | 167 | ATTAACT | 168 | 6FAM- | 169 | 98 |
| qMSP | AGTGGTTTATA | | AAACTAA | | CCTACCTTAACCTC | | |
| | TTTGTAATTTT | | TCTTAAA | | CC-MGB | | |
| | AGTA | | CTCCTAA | | | | |
| | | | CCTCA | | | | |
| CDO1 | CGAATTATAG | 170 | AAATCGC | 171 | 6FAM- | 172 | 101 |
| qMSP | CGGCGGAGGT | | GTAAACT | | CGTTAGGTCGGGCG | | |
| | | | CCGCG | | GT-MGB | | |
| DCLK1 | GCGCGTACGC | 173 | CGACGAC | 174 | 6FAM- | 175 | 86 |
| qMSP | GGAGG | | GAACGCG | | CGGGAGGGCGTGTG | | |
| | | | CT | | A-MGB | | |
| SFRP1 | GAATTCGTTCG | 176 | AAACGA | 177 | 6FAM- | 178 | 70 |
| qMSP* | CGAGGGA | | ACCGCAC | | CGTCACCGACGCGA | | |
| | | | TCGTTAC | | A-MGB | | |
| | | | C | | | | |
| ZSCA | CGCGGTATAG | 179 | CGCGATA | 180 | 6FAM- | 181 | 84 |
| N18 | TTTCGCGGTAT | | ACGACCG | | CGTAGTTCGCGGTG | | |
| qMSP | | | ACAAA | | AGG-MGB | | |

* qMSP assay was modified from Rawson and colleagues (47).

TABLE 3
Detailed information about the described genes

| HGNC | | | | | SEQ |
|---|---|---|---|---|---|
| gene | Entrez | | | Approved gene | ID |
| symbol | gene ID | Ensembl gene ID | Aliases | name | NO |
| GLDC | 2731 | ENSG00000178445 | GCSP, | glycine | 185 |
| | | | NKH | dehydrogenase | |
| | | | | (decarboxylating) | |
| PPP1R14A | 94274 | ENSG00000167641 | CPI-17 | protein | 186 |
| | | | | phosphatase 1, | |
| | | | | regulatory | |
| | | | | (inhibitor) subunit | |
| | | | | 14A | |
| CDO1 | 1036 | ENSG00000129596 | | cysteine | 187 |
| | | | | dioxygenase, | |
| | | | | type I | |
| DCLK1 | 9201 | ENSG00000133083 | KIAA0369, | doublecortin-like | 188 |
| | | | DCLK, | kinase 1 | |
| | | | DCDC3A | | |
| ZSCAN18 | 65982 | ENSG00000121413 | FLJ12895 | zinc finger and | 190 |
| | | | | SCAN domain | |
| | | | | containing 18 | |

TABLE 4
Overview of hypermethylated genes previously identified in CCA

| | | | | | Number of | |
|---|---|---|---|---|---|---|
| Gene | | | Sensitivity | Spesificity | CCAs | |
| symbol | Location | Function | (%) | (%) | analyzed | Refs |
| APC | 5q21 | Cell | 26-46 | 100, 90 | 72-111 | (55-57) |
| | | adhesion | | | | |
| BCL2 | 18q21 | Apoptosis | 23 | 97 | 111 | (55) |
| CACNA1G | 17q22 | Ion channel | 4 | 94 | 111 | (55) |
| CDH1 | 16q22 | Cell | 22-43 | 100, 90 | 15-111 | (55-59) |
| | | adhesion | | | | |
| CDKN2A | 9p21 | Cell cycle | 9-38 | 100 | 51-111 | (55-57; 60) |
| (p14^{ARF}) | | regulation | | | | |
| CDKN2A | 9p21 | Cell cycle | 14-77 | 100, 90 | 7-111 | (55-62) |
| (p16^{INK4a}) | | regulation | | | | |
| CDKN2B | 9p21 | Cell cycle | 51 | 100 | 72 | (57) |
| (p15^{INK4b}) | | regulation | | | | |
| CHFR | 12q24 | Apoptosis | 5, 17 | 100 | 23, 111 | (55; 59) |
| DAPK1 | 9q34 | Apoptosis | 3-40 | 100, 93 | 15-79 | (56-59; 63) |
| FHIT | 3p14 | Purine | 42 | 89 | 19 | (64) |
| | | metabolism | | | | |
| GSTP1 | 11q13 | Drug | 6-18 | 100, 90 | 72-111 | (55-57) |
| | | metabolism | | | | |
| HOXA1 | 7p15.2 | Development | 90 | 95 | 111 | (55) |
| IGF2 | 11p15.5 | Cell growth | 23 | 89 | 111 | (55) |
| | | and | | | | |
| | | differentiation | | | | |
| MGMT | 10q26 | DNA repair | 4-33 | 100 | 15-111 | (55-58) |
| MINT1 | 22q11 | Unknown | 38, 41 | 100 | 79, 111 | (55; 56) |
| MINT12 | 22q11 | Unknown | 51 | 100 | 79 | (56) |
| MINT2 | 22q11 | Unknown | 0, 7 | 100 | 79, 111 | (55; 56) |
| MINT25 | 22q11 | Unknown | 15 | 100 | 79 | (56) |
| MINT31 | 22q11 | Unknown | 1, 15 | 100 | 79, 111 | (55; 56) |
| MINT32 | 22q11 | Unknown | 35 | 100 | 79 | (56) |
| MLH1 | 3p22 | DNA repair | 13-47 | 100 | 15-72 | (57-59) |
| NEUROG1 | 5q23 | Cell | 53 | 89 | 111 | (55) |
| | | differentiation | | | | |
| PTGS2 | 1q25 | Biosynthesis | 5 | 100 | 79 | (56) |
| | | in | | | | |
| | | inflammation | | | | |
| PYCARD | 16p11 | Apoptosis | 36 | 92 | 36 | (65) |
| RARB | 3p24 | Cell growth | 14-18 | 100 | 72, 111 | (55; 57) |
| | | and | | | | |
| | | differentiation | | | | |
| RARRES1 | 3q25 | Membrane | 22 | 97 | 111 | (55) |
| | | protein | | | | |
| RASSF1 | 3p21 | Cell cycle | 28-73 | 50-100 | 13-111 | (55; 57; 59; |
| | | regulation | | | | 64; 66; 67) |
| RBP1 | 3q21 | Retinol | 14 | 100 | 111 | (55) |
| | | transport | | | | |
| RUNX3 | 1p36 | Apoptosis | 33, 78 | 100 | 23,111 | (55; 59) |
| SEMA3B | 3p21 | Apoptosis | 100 | 100 | 15 | (66) |
| SFN | 1p36 | Apoptosis | 59 | 100 | 79 | (56) |
| SOCS3 | 17q25 | Cytokine | 88 | 100 | 8 | (68) |
| | | signaling | | | | |
| THBS1 | 15q15 | Cell | 2-11 | 100 | 79, 111 | (55; 56) |
| | | adhesion | | | | |
| TIMP3 | 22q12 | Cell | 1-9 | 100 | 79-111 | (55; 56) |
| | | adhesion | | | | |
| TMEFF2 | 2q32.3 | Cell growth | 73 | 92 | 111 | (55) |
| | | and | | | | |
| | | differentiation | | | | |
| TP73 | 1p36 | Cell cycle | 36 | 100 | 72 | (57) |
| | | regulation | | | | |
| ZMYND10 | 3p21 | Unknown | 20 | 100 | 15 | (66) |

Genes are listed according to approved gene symbols (HUGO Gene Nomenclature Committee). Genes reported to be unmethylated are not included in the table.

TABLE 5
Culturing conditions of cancer cell lines

| Cell | | |
|---|---|---|
| line | Medium | Additives |
| TFK-1 | Roswell Park Memorial Institute (RPMI) 1640 | Penicillin-Streptomycin- |
| | medium* | Glutamine*, Fetal Bovine Serum* |
| EGI-1 | Dulbecco's Modified Eagle Medium (DMEM)* | Penicillin-Streptomycin- |
| | | Glutamine*, Fetal Bovine Serum* |
| HuCCT1 | Roswell Park Memorial Institute (RPMI) 1640 | Penicillin-Streptomycin- |
| | medium* | Glutamine*, Fetal Bovine Serum* |
| SK- | Minimum Essential Medium (MEM)* | Penicillin-Streptomycin- |
| ChA-1 | | Glutamine*, Fetal Bovine Serum* |
| Mz- | Roswell Park Memorial Institute (RPMI) 1640 | Penicillin-Streptomycin- |
| ChA-1 | medium* | Glutamine*, Fetal Bovine Serum* |
| Mz- | Minimum Essential Medium (MEM)* | Penicillin-Streptomycin- |
| ChA-2 | | Glutamine*, Fetal Bovine Serum* |
| KMCU | Dulbecco's Modified Eagle Medium (DMEM)* | Penicillin-Streptomycin- |
| | | Glutamine*, Fetal Bovine Serum* |
| KMBC | Dulbecco's Modified Eagle Medium (DMEM)* | Penicillin-Streptomycin- |
| | | Glutamine*, Fetal Bovine Serum* |
| | | and Horse Serum* |
| PaCa-2 | Dulbecco's Modified Eagle Medium (DMEM)* | Penicillin-Streptomycin- |
| | | Glutamine*, Fetal Bovine Serum* |
| | | and MEM Non Essential Amino |
| | | Acids* |
| HPAFII | Minimum Essential Medium (MEM)* | Penicillin-Streptomycin- |
| | | Glutamine*, Fetal Bovine Serum* |
| BxBc-3 | Roswell Park Memorial Institute (RPMI) 1640 | Penicillin-Streptomycin- |
| | medium* | Glutamine*, Fetal Bovine Serum* |
| AsPc-1 | Roswell Park Memorial Institute (RPMI) 1640 | Penicillin-Streptomycin- |
| | medium* | Glutamine*, Fetal Bovine Serum* |
| SW48 | Dulbecco's Modified Eagle Medium: Nutrient | Penicillin-Streptomycin- |
| | Mixture F-12 (D-MEM/F12)* | Glutamine*, Fetal Bovine Serum* |
| SW480 | Dulbecco's Modified Eagle Medium: Nutrient | Penicillin-Streptomycin- |
| | Mixture F-12 (D-MEM/F12)* | Glutamine*, Fetal Bovine Serum* |
| RKO | Dulbecco's Modified Eagle Medium: Nutrient | Penicillin-Streptomycin- |
| | Mixture F-12 (D-MEM/F12)* | Glutamine*, Fetal Bovine Serum* |
| HCT15 | Dulbecco's Modified Eagle Medium: Nutrient | Penicillin-Streptomycin- |
| | Mixture F-12 (D-MEM/F12)* | Glutamine*, Fetal Bovine Serum* |
| LS1034 | Dulbecco's Modified Eagle Medium: Nutrient | Penicillin-Streptomycin- |
| | Mixture F-12 (D-MEM/F12)* | Glutamine*, Fetal Bovine Serum* |
| HT29 | Dulbecco's Modified Eagle Medium: Nutrient | Penicillin-Streptomycin- |
| | Mixture F-12 (D-MEM/F12)* | Glutamine*, Fetal Bovine Serum* |
| HB8065 | Minimum Essential Medium (MEM)* | Penicillin-Streptomycin- |
| | | Glutamine*, Fetal Bovine Serum* |
| JHH-1 | William's Medium E* | Penicillin-Streptomycin- |
| | | Glutamine*, Fetal Bovine Serum* |
| JHH-4 | Minimum Essential Medium (MEM)* | Penicillin-Streptomycin- |
| | | Glutamine*, Fetal Bovine Serum* |
| JHH-5 | William's Medium E* | Penicillin-Streptomycin- |
| | | Glutamine*, Fetal Bovine Serum* |
| Panc-1 | Dulbecco's Modified Eagle Medium (DMEM)* | Penicillin-Streptomycin- |
| | | Glutamine*, Fetal Bovine Serum* |
| CFPAC-1 | Iscove's Modified Dulbecco's Medium | Penicillin-Streptomycin- |
| | (IMDM)^{#} | Glutamine*, Fetal Bovine Serum* |

Medium and additives were added according to requirements for each cell line.
*Gibco, Invitrogen, Carlsbad, CA, USA
^{#}ATCC, Manassas, VA, USA

TABLE 6
Cancer cell line genotypes

| Cell | AM | CSF | D13 | D16 | D18 | D19 | D21 | D2S | D3S | D5S | D7S | D8S | FG | TH | TP | |
|---|---|---|---|---|---|---|---|---|---|---|---|---|---|---|---|---|
| line | EL | 1PO | S317 | S539 | S51 | S433 | S11 | 1338 | 1358 | 818 | 820 | 1179 | A | 01 | OX | vWA |
| KMBC | X | 12 | 11 | 10 | 13 | 14, | 29 | 17 | 15 | 12, | 10, | 10, | 22 | 9.3 | 8, | 16 |
| | | | | | | 15.2 | | | | 13 | 12 | 14 | | | 11 | |
| KMCH- | XY | 11 | 10 | 9 | 14, | 13, | 30, | 22, | 16 | 10, | 10, | 10, | 22, | 9 | 8, | 14, |
| 1 | | | | | 17 | 14.2 | 32.2 | 25 | | 12 | 12 | 16 | 23 | | 11 | 18, |
| | | | | | | | | | | | | | | | | 19 |
| SK- | X | 12, | 11, | 9, | 16 | 13.2, | 28 | 25 | 15 | 11, | 10 | 13, | 25 | 6 | 8 | 14, |
| ChA-1 | | 13 | 12 | 13 | | 14 | | | | 13 | | 14 | | | | 18 |
| Mz- | X | 11 | 8, | 11, | 14 | 13, | 28 | 16, | 15 | 11 | 13, | 13 | 20, | 7 | 8 | 15, |
| ChA-1 | | | 10 | 12 | | 15 | | 24 | | | 14 | | 23 | | | 19 |
| Mz- | X | 12 | 12, | 11 | 15 | 13, | 28, | 17, | 18 | 10 | 11, | 13 | 22, | 8 | 8, | 15, |
| ChA-2 | | | 13 | | | 16 | 30 | 26 | | | 12 | | 24 | | 11 | 17 |

All genotypes for non-commercial cell lines were obtained using the AmpFLSTR Identifier PCR Amplification Kit (Applied Biosystems). The size of the analyzed short tandem repeats (STR) as detected by PCR and subsequent fragment analysis are shown for each loci. Amelogenin (Amel) is a gender-determining locus.

### Results

### Identification of Candidate Genes for Promoter DNA Hypermethylation in Cholangiocarcinomas

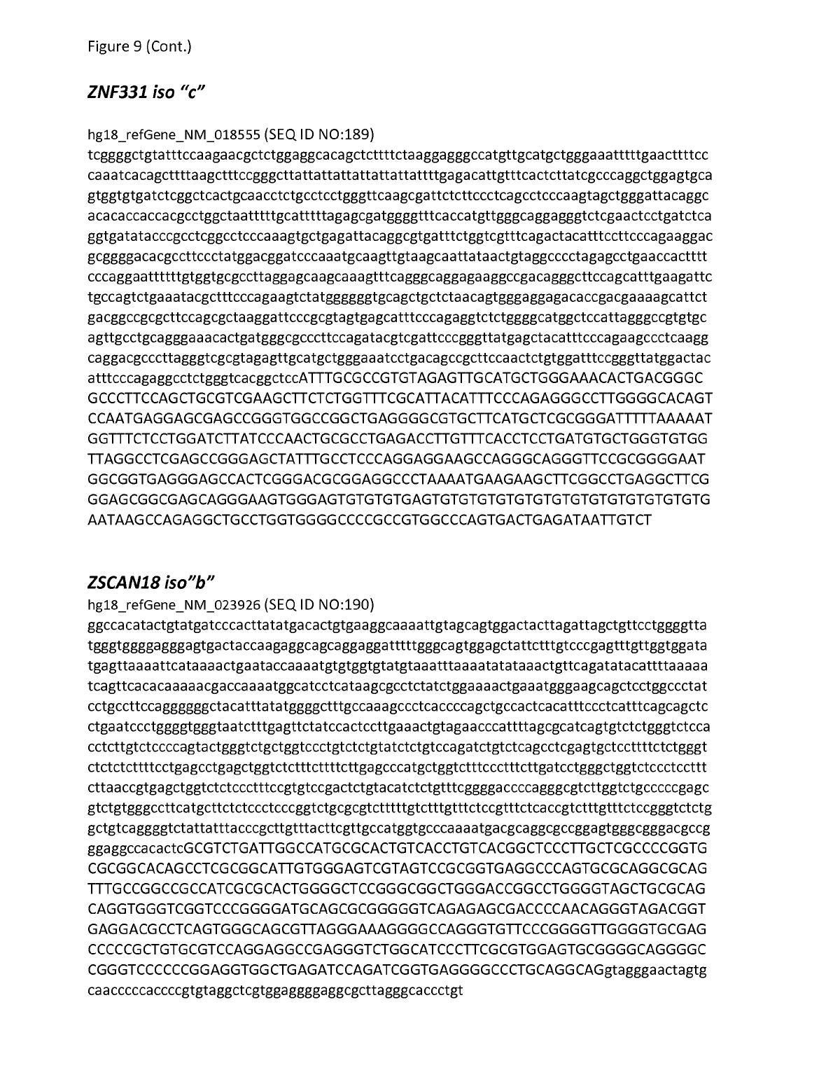

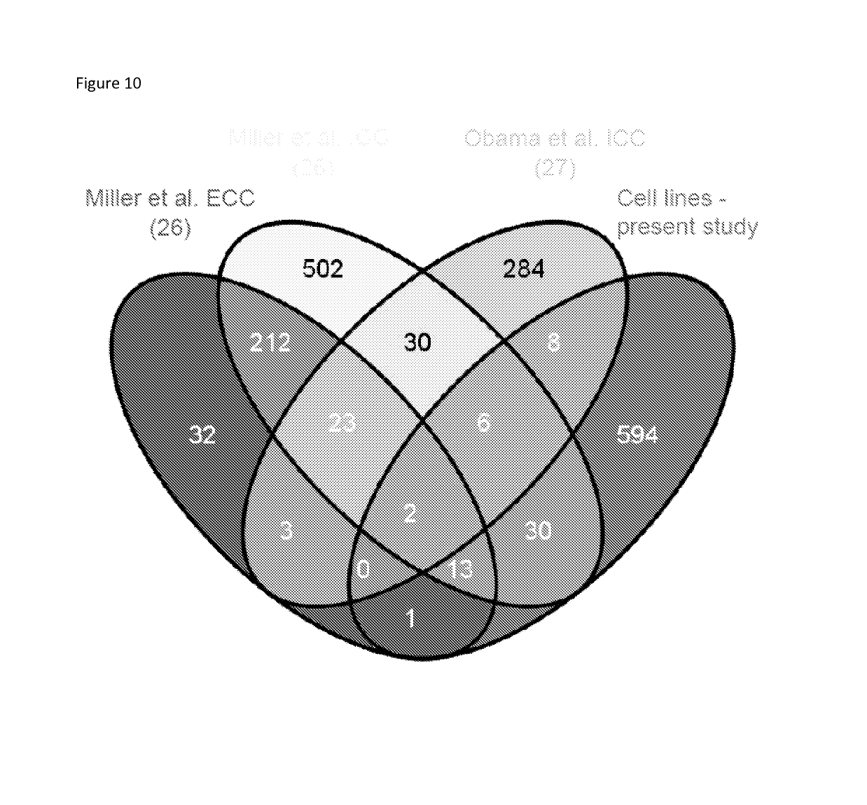

From gene expression microarray analysis we observed 672 array-elements that were up-regulated two-fold or more in a minimum of four out of the six CCA cell lines after epigenetic drug treatment (5-aza-2′deoxycytidine and trichostatin A). Sixty of these genes were simultaneously found to be down-regulated in CCA samples compared with non-malignant controls in previously published data sets (26;27) (FIG. 10). A CpG island was found in the promoter region of 43 of the candidates (and four isoforms from three of these candidates) and were regarded as potential DNA methylation target genes.

### DNA Promoter Methylation Analyses of Candidate Genes in Cancer Cell Lines

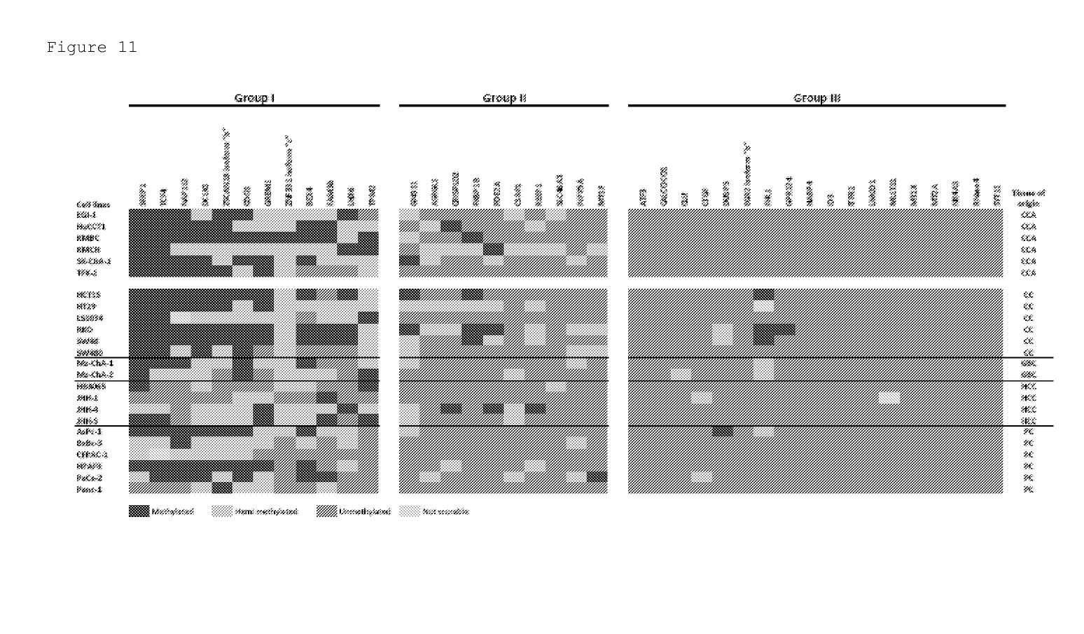

The promoter methylation status of 40 loci was investigated in 24 cancer cell lines using MSP, and grouped according to their methylation frequency in CCA cell lines (FIG. 11). Genes in group I (n=12) were frequently methylated (>⅚; BEX4, CDO1, DCLK1, FAM3B, GREM1, LHX6, NAP1L2, SFRP1, TCF4, TPM2, ZNF331 “isoform c”, and ZSCAN18 “isoform b”). Genes in group II (n=10) displayed intermediate methylation frequencies (⅙- 4/6; ASRGL1, CRISPLD2, CSRP1, FKBP1B, GNG11, INPP5A, MT1F, PDE2A, REEP1, and SLC46A3). The remaining 18 genes (group III) were unmethylated in all CCA cell lines (ATF3, CALCOCO1, CLU, CTGF, DUSP5, EGR2 “isoform b”, FHL1, GPR124, HABP4, ID3, ITPR1, LMCD1, MLLT11, MT1X, MT2A, NR4A3, RNase4, and SYT11). Genes CXCL14, DPYSL3, EGR2 “isoform a”, STXBP1, ZNF331 “isoform a”, ZNF331 “isoform b”, and ZSCAN18 “isoform a”, were excluded from further analysis based on the presence of a weak band in one of the following control reactions; the methylated reaction using normal blood, the unmethylated reaction using completely methylated DNA, or the methylated reaction using non-bisulfite treated DNA.

Interestingly, the methylation frequencies within groups I, II, and III seemed comparable among the gastrointestinal cancer cell lines included in the present study, with the exception of LHX6 and NAP1L2 which displayed little or no methylation in cell lines from hepatocellular carcinoma, and TPM2 and ZNF331 “isoform c” which showed no to little methylation in cell lines from pancreatic cancer.

### Qualitative DNA Promoter Methylation Analysis of Target Genes in Tissue Samples

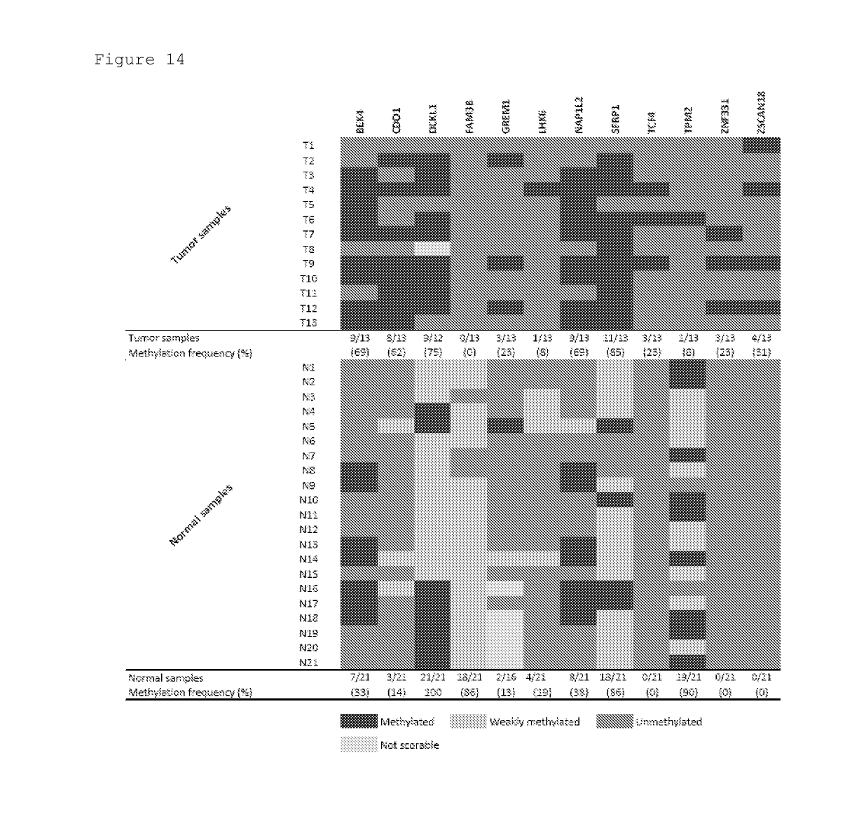

All group I genes were subjected to MSP analysis in 13 CCA and 21 non-malignant fresh-frozen clinical samples. We observed methylation of BEX4, CDO1, DCLK1, GREM1, NAP1L2, SFRP1, TCF4, ZNF331, and ZSCAN18 in 69%, 62%, 83%, 23%, 69%, 85%, 23%, 23%, and 31% in tumors and 33%, 14%, 100%, 13%, 38%, 86%, 0%, 0%, and 0% in non-malignant controls (FIG. 14). The remaining three loci, FAM3B, LHX6, and TPM2, from group I had low methylation frequencies (<10%) in tumors and varying methylation (19-90%) in the non-malignant controls. The methylated band intensities in the non-malignant controls were considerably weaker compared to tumors, indicating that a quantitative methylation analysis might discriminate more accurately between these groups. Subsequently, gene promoters exhibiting more than 30% methylation in tumors (CDO1, DCLK1, SFRP1, and ZSCAN18) were subjected to quantitative methylation analysis (qMSP). BEX4 and NAP1L2 were excluded from further analysis since they displayed methylation in female normal blood controls. Normal blood controls were included in testing of each assay)

### Validation of Promoter Methylation Status by Direct Bisulfite Sequencing

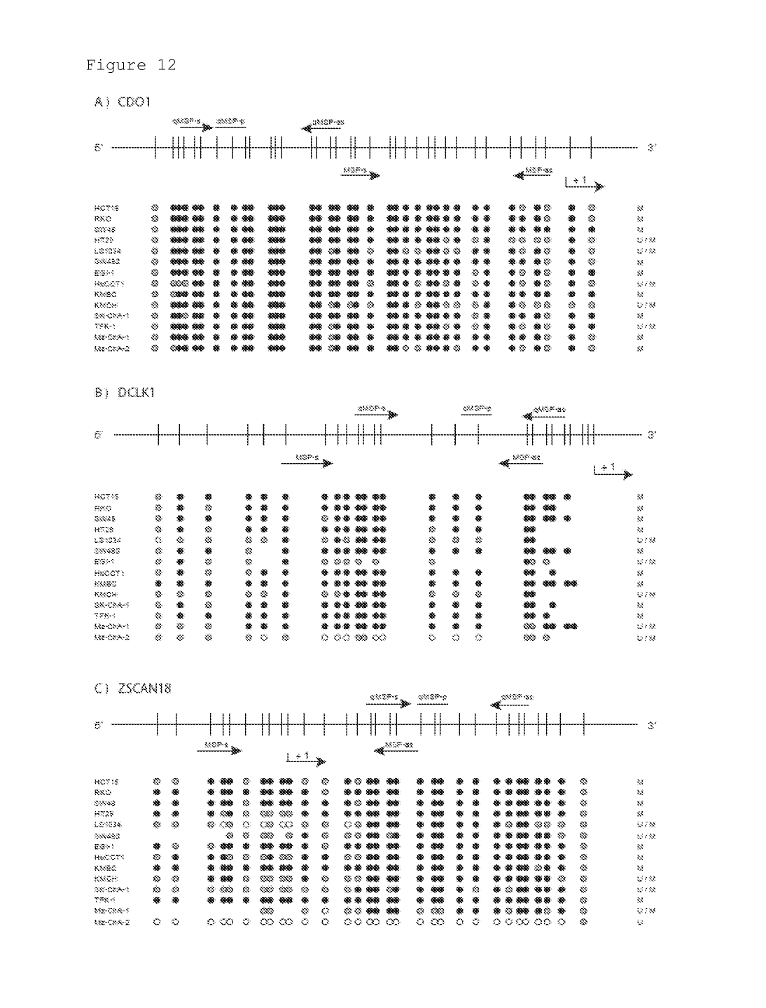

To verify the promoter methylation status as assessed by MSP, the promoter region of CDO1, DCLK1, and ZSCAN18 were subjected to direct bisulfite sequencing in representative cancer cell lines. A good concordance was seen between the MSP and bisulfite sequencing results (FIG. 12). The results were used to guide the design of the quantitative DNA methylation assays. SFRP1 has previously been analyzed by qMSP and was therefore not included in the bisulfite sequencing analysis.

### Quantitative DNA Methylation Analyses in Fresh Frozen and Archival Clinical Material

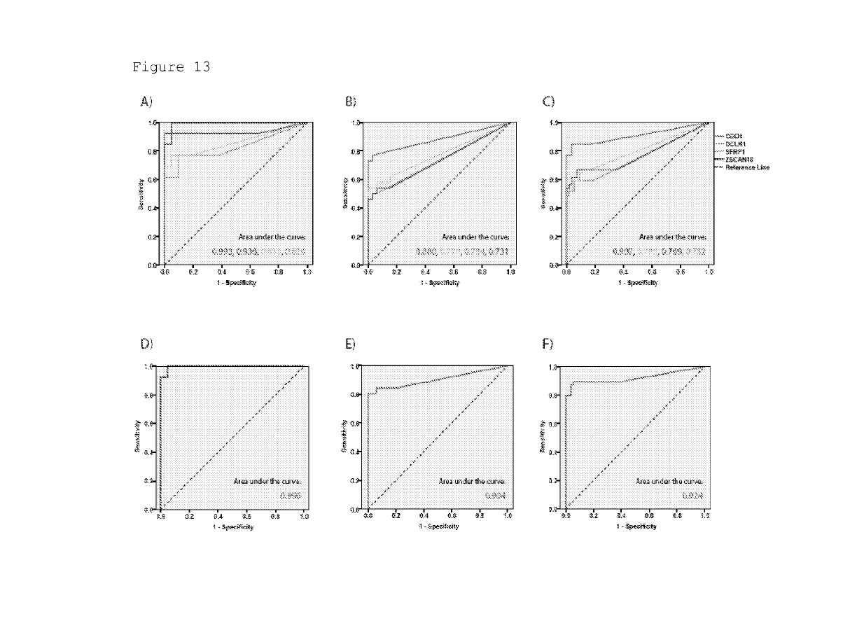

CDO1, DCLK1, SFRP1, and ZSCAN18 were analyzed with qMSP in two sample series; fresh frozen material comprising 13 CCAs and 21 non-malignant controls; and archival material comprising 26 tumor and 33 non-malignant controls. For the fresh frozen sample series, we detected promoter hypermethylation in 46%, 69%, 77%, and 85%, for DCLK1, SFRP1, ZSCAN18, and CDO1, respectively in tumors and no methylation in the non-malignant controls. By combining all four genes and scoring samples with methylation in minimum one out of the four genes as positive, 100% of the tumors and 0% non-malignant samples were methylation positive. The individual and combined performance of these genes was investigated by receiver operating characteristics curves (FIG. 13). Combining the four genes (summarizing the PMR values) resulted in an area under the curve of 0.996.

Frequencies of methylation in the archival series were in general lower than the findings in the fresh frozen sample set, although not statistically significant. The four genes DCLK1, ZSCAN18, SFRP1, and CDO1 displayed promoter methylation frequencies of 42%, 42%, 54%, and 73% in tumors, whereas no methylation was observed in non-malignant controls. The combined panel was methylation positive in 81% of the tumors. The resulting area under the curve for this sample set was 0.904 (FIG. 13).

We further investigated the methylation frequency of the four genes DCLK1, ZSCAN18, SFRP1, and CDO1 in a non-malignant sample set of six fresh frozen dissected bile duct samples with primary sclerosing cholangitis. Interestingly, two samples (33%) were methylation positive for all four biomarkers, and one sample (17%) was methylation positive for ZSCAN18.

### Discussion

In the present study, we have identified CDO1, DCLK1, and ZSCAN18 as novel frequently methylated genes in cholangiocarcinoma, in addition to the previously reported SFRP1 gene (28;29). Tissue samples from carcinoma-free individuals were unmethylated for the same genes, indicating that the promoter hypermethylation was tumor specific. The high sensitivity and specificity of these genes suggest that CDO1, DCLK1, and ZSCAN18 individually represent novel and promising biomarkers for cholangiocarcinoma. By including SFRP1, the combined biomarker panel had a sensitivity and specificity of 100% in fresh frozen material. Although the sensitivity decreased somewhat in archival material, the present biomarker panel has the potential to improve the diagnostic accuracy of CCA compared with existing clinical approaches (4;7;8).

In accordance with a previous study from our group (25) we used a step-wise experimental approach to identify novel methylation biomarkers. The approach included strict selection criteria in order to minimize the probability of selecting false positive methylation candidates. From the microarray analysis of epigenetic drug treated cancer cell lines, only genes up-regulated at least two-fold in a minimum of four of the six cholangiocarcinoma cell lines were selected. The gene expression of these candidates was further examined in available microarray data sets (26;27) and only genes down-regulated in primary CCA tumors compared with cancer-free controls were selected for further studies and subjected to promoter methylation analysis in cancer cell lines. Frequently methylated loci were subsequently analyzed in patient material and three genes (CDO1, DCLK1, and SFRP1) had methylation frequencies above 50% in tumor samples from qualitative methylation analysis. These genes, including ZSCAN18, were further subjected to a quantitative methylation analysis, resulting in 100% sensitivity and specificity for the combined biomarker panel in fresh frozen tissue.

The combined biomarker panel was methylated in 100% of cholangiocarcinomas and unmethylated across all non-malignant control samples. To increase the number of cancer samples we included a second sample series consisting of archival formalin fixed and paraffin embedded samples. As expected, the biomarker panel performance in the archival material was somewhat poorer than for the fresh frozen samples. However, the combined sensitivity (87%) and specificity (100%) for both sample series display a high performance of this biomarker panel.

Furthermore, the biomarker panel was evaluated in six non-malignant dissected bile duct samples with primary sclerosing cholangitis. Interestingly, two samples displayed positive promoter methylation in all four biomarkers. Due to positive methylation detected by this biomarker panel, we hypothesize that these findings may indicate cancer development at an early stage which may have avoided detection due to the current challenges in CCA diagnosis. Thus, this biomarker panel could add positive value to the detection of CCA in a clinical setting.

In order to detect CCA irrespective of sub-classification and disease background, the tumor material used in this study included both extrahepatic and intrahepatic lesions with and without a primary sclerosing cholangitis. A pool of benign liver diseases with biopsies taken from different locations in the liver served as controls. This was done to avoid a potential skewedness when comparing bile duct epithelial derived tumors with non-malignant samples devoid of biliary epithelium.

Epigenome-wide expression profiling has previously been used to identify potential epigenetic markers in several tumor types, including bladder, pancreas, and prostate (30-32). Using a similar experimental protocol as presented in this study (25), we have previously identified epigenetic markers for early detection of colorectal cancer (33;34). From cell line analyses we observed that these markers were present also in other cancers of the gastrointestinal tract, although at different frequencies (34). Cell lines from several hepatopancreatobiliary tumors as well as colon cancer were therefore included in the present study. We observed similarities in methylation frequencies across the majority of cancer cell lines, indicating that the genes may be aberrantly methylated also in tissue samples from other gastrointestinal tumors.

One of the four biomarkers presented here, SFRP1, has previously been investigated as a potential epigenetic biomarker in several cancers, including CCA (28;29;35;36). Secreted frizzled-related protein (SFRP) family members act as modulators of the Wnt-pathway, and hypermethylation of these promoter regions can lead to deregulation of this pathway and subsequent cancer development (37). The methylation frequencies published for SFRP1 by Uhm et al. (29), and Sriraksa et al. (28) (64%) are in the same range as presented here.

Cysteine dioxygenase, type 1 (CDO1) is reported to be highly expressed in the liver (38). It is involved in initiation of metabolic pathways related to pyruvate and sulfurate compounds, including taurine which is a major constituent of bile. CDO1 promoter hypermethylation was recently shown to be a strong marker for distant metastasis in lymph node positive, estrogen receptor positive breast cancer patients (39). In addition, CDO1 has been indicated to be epigenetically deregulated in colorectal cancer, lung cancer, and Wilms tumor (40-42). Our results support these findings and show for the first time that the promoter hypermethylation of CDO1 also may play a role in CCA.

Doublecortin like kinase 1 (DCLK1) is a microtubule-associated kinase that can undergo autophosphorylation. To our knowledge, this is the first study reporting promoter hypermethylation of this gene in cancer. However, previous studies have proposed the DCLK1 protein expression as a marker for intestinal stem cells with a role in the epithelial-mesenchymal transition (43;44). Promoter hypermethylation suggests a silencing of this gene, and further studies should be performed to clarify the role of aberrantly regulated DCLK1 in CCA.

Morris and colleagues recently reported on a putative tumor suppressor function of zinc finger and SCAN domain-containing protein 18 (ZSCAN18) in renal cell carcinoma (45). In the present study we show that ZSCAN18 is slightly more frequently methylated in cholangiocarcinomas compared with renal cancer (32%).

The number of samples analyzed represents a limitation to the present study. An increase in the number of fresh frozen and/or archival tumor- and normal samples would increase the statistical power in evaluating the presented biomarker panel. Validation in a larger sample set is therefore warranted.

Summarized, we have identified four hypermethylated genes (CDO1, DCLK1, SFRP1, and ZSCAN18) of which three (CDO1, DCLK1, and ZSCAN18) have not previously been described in CCA. The combined performance of this biomarker panel reached an 87% sensitivity and 100% specificity across fresh frozen and archival material. Further studies should be performed in minimal invasive samples, e.g. bile, biliary brush cytology specimens and/or blood in order to evaluate if the presented panel can contribute in non- to minimally-invasive CCA diagnostics.

### REFERENCES

(1) Khan S A, Thomas H C, Davidson B R, Taylor-Robinson S D. Cholangiocarcinoma. Lancet 2005; 366:1303-14.
(2) Shaib Y H, Davila J A, McGlynn K, El-Serag H B. Rising incidence of intrahepatic cholangiocarcinoma in the United States: a true increase? J Hepatol 2004; 40:472-7.
(3) Burak K, Angulo P, Pasha T M, Egan K, Petz J, Lindor K D. Incidence and risk factors for cholangiocarcinoma in primary sclerosing cholangitis. Am J Gastroenterol 2004; 99:523-6.
(4) Boberg K M, Bergquist A, Mitchell S, Pares A, Rosina F, Broome U, Chapman R et al. Cholangiocarcinoma in primary sclerosing cholangitis: risk factors and clinical presentation. Scand J Gastroenterol 2002; 37:1205-11.
(5) Sripa B, Pairojkul C. Cholangiocarcinoma: lessons from Thailand. Curr Opin Gastroenterol 2008; 24:349-56.
(6) Cai W K, Sima H, Chen B D, Yang G S. Risk factors for hilar cholangiocarcinoma: a case-control study in China. World J Gastroenterol 2011; 17:249-53.
(7) Campbell W L, Ferris J V, Holbert B L, Thaete F L, Baron R L. Biliary tract carcinoma complicating primary sclerosing cholangitis: evaluation with CT, cholangiography, US, and MR imaging. Radiology 1998; 207:41-50.
(8) Bonney G K, Craven R A, Prasad R, Melcher A F, Selby P J, Banks R E. Circulating markers of biliary malignancy: opportunities in proteomics? Lancet Oncol 2008; 9:149-58.
(9) Nehls O, Gregor M, Klump B. Serum and bile markers for cholangiocarcinoma. Semin Liver Dis 2004; 24:139-54.
(10) Akdogan M, Sasmaz N, Kayhan B, Biyikoglu I, Disibeyaz S, Sahin B Extraordinarily elevated CA19-9 in benign conditions: a case report and review of the literature. Tumori 2001; 87:337-9.
(11) Steinberg W. The clinical utility of the CA 19-9 tumor-associated antigen. Am J Gastroenterol 1990; 85:350-5.
(12) Vestergaard E M, Hein H O, Meyer H, Grunnet N, Jorgensen J, Wolf H, Orntoft T F. Reference values and biological variation for tumor marker CA 19-9 in serum for different Lewis and secretor genotypes and evaluation of secretor and Lewis genotyping in a Caucasian population. Clin Chem 1999; 45:54-61.
(13) Jones P A, Baylin S B. The fundamental role of epigenetic events in cancer. Nat Rev Genet. 2002; 3:415-28.
(14) Markowitz S D, Bertagnolli M M. Molecular origins of cancer: Molecular basis of colorectal cancer. N Engl J Med 2009; 361:2449-60.
(15) Hanahan D, Weinberg R A. Hallmarks of cancer: the next generation. Cell 2011; 144:646-74.
(16) Hatziapostolou M, Iliopoulos D. Epigenetic aberrations during oncogenesis. Cell Mol Life Sci 2011; 68:1681-702.
(17) Brooks J, Cairns P, Zeleniuch-Jacquotte A. Promoter methylation and the detection of breast cancer. Cancer Causes Control 2009; 20:1539-50.
(18) Suzuki H, Tokino T, Shinomura Y, Imai K, Toyota M. DNA methylation and cancer pathways in gastrointestinal tumors. Pharmacogenomics 2008; 9:1917-28.
(19) Bird A. DNA methylation patterns and epigenetic memory. Genes Dev 2002; 16:6-21.
(20) Chan A O, Broaddus R R, Houlihan P S, Issa J P, Hamilton S R, Rashid A. CpG island methylation in aberrant crypt foci of the colorectum. Am J Pathol 2002; 160:1823-30.
(21) Baylin S B, Herman J G, Graff J R, Vertino P M, Issa J P. Alterations in DNA methylation: a fundamental aspect of neoplasia. Adv Cancer Res 1998; 72:141-96.
(22) Belinsky S A. Gene-promoter hypermethylation as a biomarker in lung cancer. Nat Rev Cancer 2004; 4:707-17.
(23) Fujiwara K, Fujimoto N, Tabata M, Nishii K, Matsuo K, Hotta K, Kozuki T et al. Identification of epigenetic aberrant promoter methylation in serum DNA is useful for early detection of lung cancer. Clin Cancer Res 2005; 11:1219-25.
(24) Park S Y, Kwon H J, Lee H E, Ryu H S, Kim S W, Kim J H, Kim I A et al. Promoter CpG island hypermethylation during breast cancer progression. Virchows Arch 2011; 458:73-84.
(25) Lind G E, Kleivi K, Meling G I, Teixeira M R, Thiis-Evensen E, Rognum T O, Lothe R A. ADAMTS1, CRABP1, and NR3C1 identified as epigenetically deregulated genes in colorectal tumorigenesis. Cell Oncol 2006; 28:259-72.
(26) Miller G, Socci N D, Dhall D, D'Angelica M, DeMatteo R P, Allen P J, Singh B et al. Genome wide analysis and clinical correlation of chromosomal and transcriptional mutations in cancers of the biliary tract. J Exp Clin Cancer Res 2009; 28:62.
(27) Obama K, Ura K, Li M, Katagiri T, Tsunoda T, Nomura A, Satoh S et al. Genome-wide analysis of gene expression in human intrahepatic cholangiocarcinoma. Hepatology 2005; 41:1339-48.
(28) Sriraksa R, Zeller C, El-Bahrawy M A, Dai W, Daduang J, Jearanaikoon P, Chau-In S et al. CpG-island methylation study of liver fluke-related cholangiocarcinoma. Br J Cancer 2011; 104:1313-8.
(29) Uhm K O, Lee E S, Lee Y M, Kim H S, Park Y N, Park S H. Aberrant promoter CpG islands methylation of tumor suppressor genes in cholangiocarcinoma. Oncol Res 2008; 17:151-7.
(30) Costa V L, Henrique R, Danielsen S A, Duarte-Pereira S, Eknaes M, Skotheim R1, Rodrigues A et al. Three epigenetic biomarkers, GDF15, TMEFF2, and VIM, accurately predict bladder cancer from DNA-based analyses of urine samples. Clin Cancer Res 2010; 16:5842-51.
(31) Lodygin D, Epanchintsev A, Menssen A, Diebold J, Hermeking H. Functional epigenomics identifies genes frequently silenced in prostate cancer. Cancer Res 2005; 65:4218-27.
(32) Sato N, Matsubayashi H, Abe T, Fukushima N, Goggins M. Epigenetic down-regulation of CDKN1C/p57KIP2 in pancreatic ductal neoplasms identified by gene expression profiling. Clin Cancer Res 2005; 11:4681-8.
(33) Lind G E, Ahlquist T, Lothe R A. DNA hypermethylation of MAL: a promising diagnostic biomarker for colorectal tumors. Gastroenterology 2007; 132:1631-2.
(34) Lind G E, Danielsen S A, Ahlquist T, Merok M A, Andresen K, Skotheim R1, Hektoen M et al. Identification of an epigenetic biomarker panel with high sensitivity and specificity for colorectal cancer and adenomas. Mol Cancer 2011; 10:85.
(35) Gonzalo V, Lozano J J, Munoz J, Balaguer F, Pellise M, Rodriguez de M C, Andreu M et al. Aberrant gene promoter methylation associated with sporadic multiple colorectal cancer. PLoS One 2010; 5:e8777.
(36) Huang Z H, Hu Y, Hua D, Wu Y Y, Song M X, Cheng Z H. Quantitative analysis of multiple methylated genes in plasma for the diagnosis and prognosis of hepatocellular carcinoma. Exp Mol Pathol 2011; 91:702-7.
(37) Kawano Y, Kypta R. Secreted antagonists of the Wnt signalling pathway. J Cell Sci 2003; 116:2627-34.
(38) Tsuboyama-Kasaoka N, Hosokawa Y, Kodama H, Matsumoto A, Oka J, Totani M. Human cysteine dioxygenase gene: structural organization, tissue-specific expression and downregulation by phorbol 12-myristate 13-acetate. Biosci Biotechnol Biochem 1999; 63:1017-24.
(39) Dietrich D, Krispin M, Dietrich J, Fassbender A, Lewin J, Harbeck N, Schmitt M et al. CDO1 promoter methylation is a biomarker for outcome prediction of anthracycline treated, estrogen receptor-positive, lymph node-positive breast cancer patients. BMC Cancer 2010; 10:247.
(40) Kwon Y J, Lee S J, Koh J S, Kim S H, Lee H W, Kang M C, Bae J B et al. Genome-Wide Analysis of DNA Methylation and the Gene Expression Change in Lung Cancer. J Thorac Oncol 2011.
(41) Mossman D, Scott R J. Long term transcriptional reactivation of epigenetically silenced genes in colorectal cancer cells requires DNA hypomethylation and histone acetylation. PLoS One 2011; 6:e23127.
(42) Maschietto M, Piccoli F S, Costa C M, Camargo L P, Neves J I, Grundy P E, Brentani H et al. Gene expression analysis of blastemal component reveals genes associated with relapse mechanism in Wilms tumour. Eur J Cancer 2011.
(43) Sureban S M, May R, Lightfoot S A, Hoskins A B, Lerner M, Brackett D J, Postier R G et al. DCAMKL-1 regulates epithelial-mesenchymal transition in human pancreatic cells through a miR-200a-dependent mechanism. Cancer Res 2011; 71:2328-38.
(44) Souza R F, Krishnan K, Spechler S J. Acid, bile, and CDX: the ABCs of making Barrett's metaplasia. Am J Physiol Gastrointest Liver Physiol 2008; 295:G211-G218.
(45) Morris M R, Ricketts C J, Gentle D, McRonald F, Carli N, Khalili H, Brown M et al. Genome-wide methylation analysis identifies epigenetically inactivated candidate tumour suppressor genes in renal cell carcinoma. Oncogene 2011; 30:1390-401.
(46) Kim S K, Jong H R, Kim J H, Kim M, Noh S M, Song K S, Kang G H et al. CpG methylation in exon 1 of transcription factor 4 increases with age in normal gastric mucosa and is associated with gene silencing in intestinal-type gastric cancers. Carcinogenesis 2008; 29:1623-31.
(47) Rawson J B, Manno M, Mrkonjic M, Daftary D, Dicks E, Buchanan D D, Younghusband H B et al. Promoter methylation of Wnt antagonists DKK1 and SFRP1 is associated with opposing tumor subtypes in two large populations of colorectal cancer patients. Carcinogenesis 2011; 32:741-7.
(48) Takai D, Jones P A. The CpG island searcher: a new WWW resource. In Silico Biol 2003; 3:235-40.
(49) Takai D, Jones P A. Comprehensive analysis of CpG islands in human chromosomes 21 and 22. Proc Natl Acad Sci USA 2002; 99:3740-5.
(50) Clark S J, Harrison J, Paul C L, Frommer M. High sensitivity mapping of methylated cytosines. Nucleic Acids Res 1994; 22:2990-7.
(51) Herman J G, Graff J R, Myohanen S, Nelkin B D, Baylin S B. Methylation-specific PCR: a novel PCR assay for methylation status of CpG islands. Proc Natl Acad Sci USA 1996; 93:9821-6.
(52) Lind G E, Ahlquist T, Kolberg M, Berg M, Eknaes M, Alonso M A, Kallioniemi A et al. Hypermethylated MAL gene—a silent marker of early colon tumorigenesis. J Transl Med 2008; 6:13.
(53) Melki J R, Vincent P C, Clark S J. Concurrent DNA hypermethylation of multiple genes in acute myeloid leukemia. Cancer Res 1999; 59:3730-40.
(54) Weisenberger D J, Campan M, Long T I, Kim M, Woods C, Fiala E, Ehrlich M et al. Analysis of repetitive element DNA methylation by MethyLight. Nucleic Acids Res 2005; 33:6823-36.
(55) Kim B H, Cho N Y, Choi M, Lee S, Jang J J, Kang G H. Methylation profiles of multiple CpG island loci in extrahepatic cholangiocarcinoma versus those of intrahepatic cholangiocarcinomas. Arch Pathol Lab Med 2007; 131:923-30.
(56) Lee S, Kim W H, Jung H Y, Yang M H, Kang G H. Aberrant CpG island methylation of multiple genes in intrahepatic cholangiocarcinoma. Am J Pathol 2002; 161:1015-22.
(57) Yang B, House M G, Guo M, Herman J G, Clark D P. Promoter methylation profiles of tumor suppressor genes in intrahepatic and extrahepatic cholangiocarcinoma. Mod Pathol 2005; 18:412-20.
(58) Koga Y, Kitajima Y, Miyoshi A, Sato K, Kitahara K, Soejima H, Miyazaki K. Tumor progression through epigenetic gene silencing of O(6)-methylguanine-DNA methyltransferase in human biliary tract cancers. Ann Surg Oncol 2005; 12:354-63.
(59) Tozawa T, Tamura G, Honda T, Nawata S, Kimura W, Makino N, Kawata S et al. Promoter hypermethylation of DAP-kinase is associated with poor survival in primary biliary tract carcinoma patients. Cancer Sci 2004; 95:736-40.
(60) Tannapfel A, Sommerer F, Benicke M, Weinans L, Katalinic A, Geissler F, Uhlmann D et al. Genetic and epigenetic alterations of the INK4a-ARF pathway in cholangiocarcinoma. J Pathol 2002; 197:624-31.
(61) Ahrendt S A, Eisenberger C F, Yip L, Rashid A, Chow J T, Pitt H A, Sidransky D. Chromosome 9p21 loss and p16 inactivation in primary sclerosing cholangitis-associated cholangiocarcinoma. J Surg Res 1999; 84:88-93.
(62) Hong S M, Choi J, Ryu K, Ro J Y, Yu E. Promoter hypermethylation of the p16 gene and loss of its protein expression is correlated with tumor progression in extrahepatic bile duct carcinomas. Arch Pathol Lab Med 2006; 130:33-8.
(63) Liu X F, Kong F M, Xu Z, Yu S P, Sun F B, Zhang C S, Huang Q X et al. Promoter hypermethylation of death-associated protein kinase gene in cholangiocarcinoma. Hepatobiliary Pancreat Dis Int 2007; 6:407-11.
(64) Foja S, Goldberg M, Schagdarsurengin U, Dammann R, Tannapfel A, Ballhausen W G. Promoter methylation and loss of coding exons of the fragile histidine triad
(FHIT) gene in intrahepatic cholangiocarcinomas. Liver Int 2005; 25:1202-8.
(65) Liu X F, Zhu S G, Zhang H, Xu Z, Su H L, Li S J, Zhou X T. The methylation status of the TMST/ASC gene in cholangiocarcinoma and its clinical significance. Hepatobiliary Pancreat Dis Int 2006; 5:449-53.
(66) Tischoff I, Markwarth A, Witzigmann H, Uhlmann D, Hauss J, Mirmohammadsadegh A, Wittekind C et al. Allele loss and epigenetic inactivation of 3p21.3 in malignant liver tumors. Int J Cancer 2005; 115:684-9.
(67) Wong N, Li L, Tsang K, Lai P B, To K F, Johnson P J. Frequent loss of chromosome 3p and hypermethylation of RASSF1A in cholangiocarcinoma. J Hepatol 2002; 37:633-9.
(68) Isomoto H, Mott J L, Kobayashi S, Werneburg N R, Bronk S F, Haan S, Gores G J. Sustained I L-6/STAT-3 signaling in cholangiocarcinoma cells due to SOCS-3 epigenetic silencing. Gastroenterology 2007; 132:384-96.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the medical sciences are intended to be within the scope of the following claims.

1. A method for detecting a gastrointestinal neoplasm in a subject comprising:
a) obtaining DNA from a biological sample of said subject; and
b) determining the level, presence, or frequency of methylation of a nucleic acid polymer corresponding to one or more genes selected from the group consisting of GLDC, PPP1R14A, CDO1, DCLK1, ZSCAN18 and ZNF331.

2. The method of claim 1, wherein the level, presence, or frequency of methylation of a nucleic acid polymer corresponding to at least one additional gene is determined.

3. The method of claim 2, wherein said at least one additional gene is SFRP1.

4. The method of claim 1, wherein the level or frequency of methylation of a nucleic acid polymer is compared to a reference level or frequency of methylations.

5. The method of claim 1, further comprising comparing the level, presence, or frequency of methylation of said nucleic acid polymer with a reference level, presence, or frequency of methylation, wherein an altered level, presence, or frequency of methylation for said patient relative to said reference provides an indication selected from the group consisting of an indication of a predisposition of the subject to a gastrointestinal cancer, an indication that the subject has gastrointestinal cancer, an indication of the likelihood of recurrence of gastrointestinal cancer in the subject, an indication of survival of the subject, and indication of the aggressiveness of gastrointestinal cancer, an indication of the likely outcome of treatment of gastrointestinal cancer and an indication that the subject is a candidate for treatment with a particular therapy.

6. The method of claim 1, wherein said nucleic acid comprises a region selected from the group consisting of a CpG island and a CpG island shore.

7. The method of claim 6, wherein said CpG island or shore is present in a coding region or a regulatory region.

8. The method of claim 6, wherein said regulatory region is a promoter.

9. The method of claim 6, wherein said determining the level, presence, or frequency of methylation of a nucleic acid polymer comprises determining the methylation frequency of said CpG island or island shore.

10. The method of claim 1, wherein said determining the level, presence, or frequency of methylation of a nucleic acid polymer is achieved by a technique selected from the group consisting of methylation-specific PCR, quantitative methylation-specific PCR, methylation-sensitive DNA restriction enzyme analysis, methylation—insensitive DNA restriction enzyme analysis, quantitative bisulfite pyrosequencing, and bisulfite genomic sequencing PCR.

11. The method of claim 1, further comprising: c) generating a risk profile using the results of steps a) and b).

12. The method of claim 1, wherein said gastrointestinal neoplasm is colorectal cancer, gastric cancer, pancreatic cancer, liver cancer, cancers of the gall bladder and/or bile ducts, or cholangiocarcinoma.

13. The method of claim 1, wherein said method permits detection of gastrointestinal cancer in said subject with a sensitivity of at least 85% at a specificity of at least 85%.

14. The method of claim 1, wherein said method permits detection of gastrointestinal cancer in said subject with a sensitivity of at least 80% at a specificity of at least 90%.

15. The method of claim 1, wherein said biological sample is selected from the group consisting of a tissue sample, a stool sample, a cell sample, a bile sample and a blood sample.

16. A methylation specific nucleic acid detection sequence corresponding to one or more genes selected from the group consisting of GLDC, PPP1R14A, CDO1, DCLK1, ZSCAN18 and ZNF331.

17. A method comprising contacting a nucleic acid sample from a subject with the nucleic acid sequence of claim 16 to detect a cancerous condition in a subject.

18. The method of claim 17, wherein said cancerous condition is a gastrointestinal neoplasm.

19. The method of claim 18, wherein said gastrointestinal neoplasm is colorectal cancer, gastric cancer, pancreatic cancer, liver cancer, cancers of the gall bladder and/or bile ducts, or cholangiocarcinoma.

20. The method e of claim 18 wherein the gastrointestinal neoplasm is colorectal cancer or cholangiocarcinoma.

21. The method of claim 17, wherein an additional methylation specific nucleic acid detection sequence is utilized in addition to detection sequences for one or more of GLDC, PPP1R14A, CDO1, DCLK1, ZSCAN18 and ZNF331.

22. The method of claim 21, wherein said additional methylation specific nucleic acid detection sequence corresponds to SFRP1.

23. The method of claim 17, wherein an altered level, presence, or frequency of methylation for a patient relative to a reference provides an indication selected from the group consisting of an indication of a predisposition of the subject to a gastrointestinal cancer, an indication that the subject has gastrointestinal cancer, an indication of the likelihood of recurrence of gastrointestinal cancer in the subject, an indication of survival of the subject, and indication of the aggressiveness of gastrointestinal cancer, an indication of the likely outcome of treatment of gastrointestinal cancer and an indication that the subject is a candidate for treatment with a particular therapy

24. A kit for detecting the presence of a gastrointestinal neoplasm in a mammal, said kit comprising reagents useful, sufficient, or necessary for detecting and/or characterizing level, presence, or frequency of methylation of one or more genes selected from the group consisting of GLDC, PPP1R14A, CDO1, DCLK1, ZSCAN18 and ZNF331.

25. A system comprising a computer readable medium comprising instructions for utilizing information on the level, presence, or frequency of methylation of one or more genes selected from the group consisting of GLDC, PPP1R14A, CDO1, DCLK1, ZSCAN18 and ZNF331 to provide an indication selected from the group consisting of an indication of a predisposition of the subject to a gastrointestinal cancer, an indication that the subject has gastrointestinal cancer, an indication of the likelihood of recurrence of gastrointestinal cancer in the subject, an indication of survival of the subject, and indication of the aggressiveness of gastrointestinal cancer, an indication of the likely outcome of treatment of gastrointestinal cancer and an indication that the subject is a candidate for treatment with a particular therapy.


SEQUENCE LISTING (190 sequences)

SEQ ID NO: 1 - DNA, 20 bp, Artificial Sequence
  Synthetic
gagataggtgcgcgttagtc

SEQ ID NO: 2 - DNA, 20 bp, Artificial Sequence
  Synthetic
acaacgattctacgcctacg

SEQ ID NO: 3 - DNA, 23 bp, Artificial Sequence
  Synthetic
agtgagataggtgtgtgttagtt

SEQ ID NO: 4 - DNA, 23 bp, Artificial Sequence
  Synthetic
acaacaattctacacctacacac

SEQ ID NO: 5 - DNA, 20 bp, Artificial Sequence
  Synthetic
agcgagtacgtatatttggc

SEQ ID NO: 6 - DNA, 18 bp, Artificial Sequence
  Synthetic
aaaacgaaaccgaaaacg

SEQ ID NO: 7 - DNA, 23 bp, Artificial Sequence
  Synthetic
agtagtgagtatgtatatttggt

SEQ ID NO: 8 - DNA, 21 bp, Artificial Sequence
  Synthetic
accaaaacaaaaccaaaaaca

SEQ ID NO: 9 - DNA, 21 bp, Artificial Sequence
  Synthetic
aggggttgattcgaaagtttc

SEQ ID NO: 10 - DNA, 20 bp, Artificial Sequence
  Synthetic
tctaacgccaaaacgaaaca

SEQ ID NO: 11 - DNA, 24 bp, Artificial Sequence
  Synthetic
gataggggttgatttgaaagtttt

SEQ ID NO: 12 - DNA, 23 bp, Artificial Sequence
  Synthetic
aactctaacaccaaaacaaaaca

SEQ ID NO: 13 - DNA, 20 bp, Artificial Sequence
  Synthetic
tacgttttttaggatgtcgc

SEQ ID NO: 14 - DNA, 20 bp, Artificial Sequence
  Synthetic
cttttaccgctacgtactcg

SEQ ID NO: 15 - DNA, 23 bp, Artificial Sequence
  Synthetic
aattatgttttttaggatgttgt

SEQ ID NO: 16 - DNA, 23 bp, Artificial Sequence
  Synthetic
cccttttaccactacatactcaa

SEQ ID NO: 17 - DNA, 19 bp, Artificial Sequence
  Synthetic
ttgggacgtcggagataac

SEQ ID NO: 18 - DNA, 20 bp, Artificial Sequence
  Synthetic
gaccctcgaaaaaaaaacga

SEQ ID NO: 19 - DNA, 22 bp, Artificial Sequence
  Synthetic
tttttgggatgttggagataat

SEQ ID NO: 20 - DNA, 23 bp, Artificial Sequence
  Synthetic
aaccctcaaaaaaaaaacaaaac

SEQ ID NO: 21 - DNA, 20 bp, Artificial Sequence
  Synthetic
ttttttttgtttaygtttta

SEQ ID NO: 22 - DNA, 19 bp, Artificial Sequence
  Synthetic
acaaatcaaattcaaatct

SEQ ID NO: 23 - DNA, 20 bp, Artificial Sequence
  Synthetic
ttttttttattggaagcgtc

SEQ ID NO: 24 - DNA, 19 bp, Artificial Sequence
  Synthetic
aaaaaataccgcgaaaaac

SEQ ID NO: 25 - DNA, 23 bp, Artificial Sequence
  Synthetic
ggtttttttttattggaagtgtt

SEQ ID NO: 26 - DNA, 22 bp, Artificial Sequence
  Synthetic
ccaaaaaataccacaaaaaaca

SEQ ID NO: 27 - DNA, 18 bp, Artificial Sequence
  Synthetic
ttcgtttattcggcgttc

SEQ ID NO: 28 - DNA, 19 bp, Artificial Sequence
  Synthetic
actcaacgtaccgcctctt

SEQ ID NO: 29 - DNA, 21 bp, Artificial Sequence
  Synthetic
ttttttgtttatttggtgttt

SEQ ID NO: 30 - DNA, 22 bp, Artificial Sequence
  Synthetic
aaaactcaacataccacctctt

SEQ ID NO: 31 - DNA, 20 bp, Artificial Sequence
  Synthetic
acgtgtaagacgtttttcgc

SEQ ID NO: 32 - DNA, 20 bp, Artificial Sequence
  Synthetic
aacccgacgatactaccctc

SEQ ID NO: 33 - DNA, 22 bp, Artificial Sequence
  Synthetic
gtatgtgtaagatgttttttgt

SEQ ID NO: 34 - DNA, 22 bp, Artificial Sequence
  Synthetic
aacccaacaatactaccctcct

SEQ ID NO: 35 - DNA, 20 bp, Artificial Sequence
  Synthetic
tcggagcgtataaaagtttc

SEQ ID NO: 36 - DNA, 20 bp, Artificial Sequence
  Synthetic
ctatcgaccgaaacgactac

SEQ ID NO: 37 - DNA, 22 bp, Artificial Sequence
  Synthetic
gtttggagtgtataaaagtttt

SEQ ID NO: 38 - DNA, 22 bp, Artificial Sequence
  Synthetic
ctatcaaccaaaacaactacca

SEQ ID NO: 39 - DNA, 19 bp, Artificial Sequence
  Synthetic
gcgttttgttaagaagggc

SEQ ID NO: 40 - DNA, 19 bp, Artificial Sequence
  Synthetic
acgcgctcccttttcttat

SEQ ID NO: 41 - DNA, 19 bp, Artificial Sequence
  Synthetic
gtgttttgttaagaagggt

SEQ ID NO: 42 - DNA, 19 bp, Artificial Sequence
  Synthetic
acacactcccttttcttat

SEQ ID NO: 43 - DNA, 22 bp, Artificial Sequence
  Synthetic
aagattatttgtggggattagg

SEQ ID NO: 44 - DNA, 22 bp, Artificial Sequence
  Synthetic
aacctctctctccaaaaaaaaa

SEQ ID NO: 45 - DNA, 21 bp, Artificial Sequence
  Synthetic
gagtgagttttttagcgaagc

SEQ ID NO: 46 - DNA, 20 bp, Artificial Sequence
  Synthetic
ataaataccgtccgtaacgc

SEQ ID NO: 47 - DNA, 21 bp, Artificial Sequence
  Synthetic
gagtgagttttttagtgaagt

SEQ ID NO: 48 - DNA, 20 bp, Artificial Sequence
  Synthetic
ataaataccatccataacac

SEQ ID NO: 49 - DNA, 20 bp, Artificial Sequence
  Synthetic
tatatgggtagcgacgttac

SEQ ID NO: 50 - DNA, 22 bp, Artificial Sequence
  Synthetic
tcgccgaactattaatcaatta

SEQ ID NO: 51 - DNA, 23 bp, Artificial Sequence
  Synthetic
ttatatatgggtagtgatgttat

SEQ ID NO: 52 - DNA, 25 bp, Artificial Sequence
  Synthetic
ccctcaccaaactattaatcaatta

SEQ ID NO: 53 - DNA, 20 bp, Artificial Sequence
  Synthetic
ggggaacgggtttatttttc

SEQ ID NO: 54 - DNA, 18 bp, Artificial Sequence
  Synthetic
gcgaccaatcgaacaaat

SEQ ID NO: 55 - DNA, 20 bp, Artificial Sequence
  Synthetic
ggggaatgggtttatttttt

SEQ ID NO: 56 - DNA, 18 bp, Artificial Sequence
  Synthetic
acaaccaatcaaacaaat

SEQ ID NO: 57 - DNA, 20 bp, Artificial Sequence
  Synthetic
tcgtgtagtgggtagagttc

SEQ ID NO: 58 - DNA, 18 bp, Artificial Sequence
  Synthetic
ctccgccgaacgataaat

SEQ ID NO: 59 - DNA, 23 bp, Artificial Sequence
  Synthetic
tttttgtgtagtgggtagagttt

SEQ ID NO: 60 - DNA, 21 bp, Artificial Sequence
  Synthetic
cccctccaccaaacaataaat

SEQ ID NO: 61 - DNA, 19 bp, Artificial Sequence
  Synthetic
ggttcgttaatagtcgggc

SEQ ID NO: 62 - DNA, 20 bp, Artificial Sequence
  Synthetic
ctaaaatcgaaacctacgcg

SEQ ID NO: 63 - DNA, 22 bp, Artificial Sequence
  Synthetic
ttaggtttgttaatagttgggt

SEQ ID NO: 64 - DNA, 23 bp, Artificial Sequence
  Synthetic
actaaaatcaaaacctacacaaa

SEQ ID NO: 65 - DNA, 19 bp, Artificial Sequence
  Synthetic
tcggatgtgatttggaaac

SEQ ID NO: 66 - DNA, 19 bp, Artificial Sequence
  Synthetic
cgcgaaaaacgactaaact

SEQ ID NO: 67 - DNA, 21 bp, Artificial Sequence
  Synthetic
atttggatgtgatttggaaat

SEQ ID NO: 68 - DNA, 21 bp, Artificial Sequence
  Synthetic
cccacaaaaaacaactaaact

SEQ ID NO: 69 - DNA, 18 bp, Artificial Sequence
  Synthetic
gggtttaggtttggtcgc

SEQ ID NO: 70 - DNA, 20 bp, Artificial Sequence
  Synthetic
ccgctccgtaccataaataa

SEQ ID NO: 71 - DNA, 21 bp, Artificial Sequence
  Synthetic
agagggtttaggtttggttgt

SEQ ID NO: 72 - DNA, 23 bp, Artificial Sequence
  Synthetic
ccaccactccataccataaataa

SEQ ID NO: 73 - DNA, 20 bp, Artificial Sequence
  Synthetic
agtagataaagaggcgaggc

SEQ ID NO: 74 - DNA, 19 bp, Artificial Sequence
  Synthetic
aaataccgacgacaaaacg

SEQ ID NO: 75 - DNA, 23 bp, Artificial Sequence
  Synthetic
gggagtagataaagaggtgaggt

SEQ ID NO: 76 - DNA, 22 bp, Artificial Sequence
  Synthetic
aaataccaacaacaaaacacaa

SEQ ID NO: 77 - DNA, 22 bp, Artificial Sequence
  Synthetic
cgtgacgtgatagtagtcggtc

SEQ ID NO: 78 - DNA, 19 bp, Artificial Sequence
  Synthetic
ctatccgacccctaccgac

SEQ ID NO: 79 - DNA, 23 bp, Artificial Sequence
  Synthetic
gtgtgatgtgatagtagttggtt

SEQ ID NO: 80 - DNA, 20 bp, Artificial Sequence
  Synthetic
cctatccaacccctaccaac

SEQ ID NO: 81 - DNA, 19 bp, Artificial Sequence
  Synthetic
ttcggaggagttgtggttc

SEQ ID NO: 82 - DNA, 21 bp, Artificial Sequence
  Synthetic
cgctaataccgaaaaaaaacg

SEQ ID NO: 83 - DNA, 22 bp, Artificial Sequence
  Synthetic
gattttggaggagttgtggttt

SEQ ID NO: 84 - DNA, 24 bp, Artificial Sequence
  Synthetic
cactaataccaaaaaaaaacaaac

SEQ ID NO: 85 - DNA, 20 bp, Artificial Sequence
  Synthetic
ttagcggatttaatggttgc

SEQ ID NO: 86 - DNA, 19 bp, Artificial Sequence
  Synthetic
taaccgaaactccgacctc

SEQ ID NO: 87 - DNA, 20 bp, Artificial Sequence
  Synthetic
ttagtggatttaatggttgt

SEQ ID NO: 88 - DNA, 19 bp, Artificial Sequence
  Synthetic
taaccaaaactccaacctc

SEQ ID NO: 89 - DNA, 20 bp, Artificial Sequence
  Synthetic
atttagggtttagttcgggc

SEQ ID NO: 90 - DNA, 21 bp, Artificial Sequence
  Synthetic
acactttaaaacgactccgaa

SEQ ID NO: 91 - DNA, 23 bp, Artificial Sequence
  Synthetic
tttatttagggtttagtttgggt

SEQ ID NO: 92 - DNA, 24 bp, Artificial Sequence
  Synthetic
actacactttaaaacaactccaaa

SEQ ID NO: 93 - DNA, 19 bp, Artificial Sequence
  Synthetic
tgcggttgtggtttttttc

SEQ ID NO: 94 - DNA, 18 bp, Artificial Sequence
  Synthetic
ccgaaacgacgttctcat

SEQ ID NO: 95 - DNA, 22 bp, Artificial Sequence
  Synthetic
tattgtggttgtggtttttttt

SEQ ID NO: 96 - DNA, 21 bp, Artificial Sequence
  Synthetic
acaccaaaacaacattctcat

SEQ ID NO: 97 - DNA, 20 bp, Artificial Sequence
  Synthetic
ggtagtcggcgtttagtttc

SEQ ID NO: 98 - DNA, 20 bp, Artificial Sequence
  Synthetic
cgcaactaaaccgctttaat

SEQ ID NO: 99 - DNA, 23 bp, Artificial Sequence
  Synthetic
tagggtagttggtgtttagtttt

SEQ ID NO: 100 - DNA, 23 bp, Artificial Sequence
  Synthetic
aaacacaactaaaccactttaat

SEQ ID NO: 101 - DNA, 19 bp, Artificial Sequence
  Synthetic
tttttcgggttagttttgc

SEQ ID NO: 102 - DNA, 20 bp, Artificial Sequence
  Synthetic
aaccgaacgaatttcgtaat

SEQ ID NO: 103 - DNA, 22 bp, Artificial Sequence
  Synthetic
gggttttttgggttagttttgt

SEQ ID NO: 104 - DNA, 23 bp, Artificial Sequence
  Synthetic
ccaaaccaaacaaatttcataat

SEQ ID NO: 105 - DNA, 20 bp, Artificial Sequence
  Synthetic
gtttaggggattttgcgttc

SEQ ID NO: 106 - DNA, 20 bp, Artificial Sequence
  Synthetic
acaaccgaccgctactttaa

SEQ ID NO: 107 - DNA, 20 bp, Artificial Sequence
  Synthetic
gtttaggggattttgtgttt

SEQ ID NO: 108 - DNA, 20 bp, Artificial Sequence
  Synthetic
acaaccaaccactactttaa

SEQ ID NO: 109 - DNA, 21 bp, Artificial Sequence
  Synthetic
ggtttacgggttgttgtattc

SEQ ID NO: 110 - DNA, 20 bp, Artificial Sequence
  Synthetic
aaaaaccgacgactctcttt

SEQ ID NO: 111 - DNA, 22 bp, Artificial Sequence
  Synthetic
gggtttatgggttgttgtattt

SEQ ID NO: 112 - DNA, 21 bp, Artificial Sequence
  Synthetic
caaaaaccaacaactctcttt

SEQ ID NO: 113 - DNA, 18 bp, Artificial Sequence
  Synthetic
gtgtgtagagtcgggtgc

SEQ ID NO: 114 - DNA, 21 bp, Artificial Sequence
  Synthetic
aaaaccgaaacgaatacaaaa

SEQ ID NO: 115 - DNA, 18 bp, Artificial Sequence
  Synthetic
gtgtgtagagttgggtgt

SEQ ID NO: 116 - DNA, 21 bp, Artificial Sequence
  Synthetic
aaaaccaaaacaaatacaaaa

SEQ ID NO: 117 - DNA, 21 bp, Artificial Sequence
  Synthetic
gcgtaattatattgcggtatc

SEQ ID NO: 118 - DNA, 20 bp, Artificial Sequence
  Synthetic
tacgttaaccgatcctacaa

SEQ ID NO: 119 - DNA, 24 bp, Artificial Sequence
  Synthetic
gttgtgtaattatattgtggtatt

SEQ ID NO: 120 - DNA, 23 bp, Artificial Sequence
  Synthetic
aactacattaaccaatcctacaa

SEQ ID NO: 121 - DNA, 19 bp, Artificial Sequence
  Synthetic
ttttcgtatacgcggaatc

SEQ ID NO: 122 - DNA, 21 bp, Artificial Sequence
  Synthetic
tcgacacgtcatttataccac

SEQ ID NO: 123 - DNA, 22 bp, Artificial Sequence
  Synthetic
ttttttttgtatatgtggaatt

SEQ ID NO: 124 - DNA, 24 bp, Artificial Sequence
  Synthetic
ctctcaacacatcatttataccac

SEQ ID NO: 125 - DNA, 18 bp, Artificial Sequence
  Synthetic
attaggcgaagttgtcgc

SEQ ID NO: 126 - DNA, 19 bp, Artificial Sequence
  Synthetic
cgactcgtccgacttaaaa

SEQ ID NO: 127 - DNA, 20 bp, Artificial Sequence
  Synthetic
ggattaggtgaagttgttgt

SEQ ID NO: 128 - DNA, 21 bp, Artificial Sequence
  Synthetic
aacaactcatccaacttaaaa

SEQ ID NO: 129 - DNA, 20 bp, Artificial Sequence
  Synthetic
ggacgcgttcgtttttagtc

SEQ ID NO: 130 - DNA, 19 bp, Artificial Sequence
  Synthetic
aaccgcgacacgttctaac

SEQ ID NO: 131 - DNA, 23 bp, Artificial Sequence
  Synthetic
gtaggatgtgtttgtttttagtt

SEQ ID NO: 132 - DNA, 22 bp, Artificial Sequence
  Synthetic
aaccacaacacattctaacaac

SEQ ID NO: 133 - DNA, 20 bp, Artificial Sequence
  Synthetic
taaatttcggacgagttttc

SEQ ID NO: 134 - DNA, 21 bp, Artificial Sequence
  Synthetic
tcgcgaaacaatttatatttc

SEQ ID NO: 135 - DNA, 23 bp, Artificial Sequence
  Synthetic
gtttaaattttggatgagttttt

SEQ ID NO: 136 - DNA, 24 bp, Artificial Sequence
  Synthetic
ccatcacaaaacaatttatatttc

SEQ ID NO: 137 - DNA, 21 bp, Artificial Sequence
  Synthetic
tagtaaatcgaattcgttcgc

SEQ ID NO: 138 - DNA, 19 bp, Artificial Sequence
  Synthetic
tacgcgaaactcctacgac

SEQ ID NO: 139 - DNA, 24 bp, Artificial Sequence
  Synthetic
ttttagtaaattgaatttgtttgt

SEQ ID NO: 140 - DNA, 22 bp, Artificial Sequence
  Synthetic
tacacaaaactcctacaaccaa

SEQ ID NO: 141 - DNA, 19 bp, Artificial Sequence
  Synthetic
gttgagtggttgttcggtc

SEQ ID NO: 142 - DNA, 20 bp, Artificial Sequence
  Synthetic
cccgactctcctacgattaa

SEQ ID NO: 143 - DNA, 21 bp, Artificial Sequence
  Synthetic
gtgttgagtggttgtttggtt

SEQ ID NO: 144 - DNA, 22 bp, Artificial Sequence
  Synthetic
tacccaactctcctacaattaa

SEQ ID NO: 145 - DNA, 19 bp, Artificial Sequence
  Synthetic
cgttttggaattatagcgc

SEQ ID NO: 146 - DNA, 19 bp, Artificial Sequence
  Synthetic
ttccgaataatcctcgaaa

SEQ ID NO: 147 - DNA, 22 bp, Artificial Sequence
  Synthetic
ttttgttttggaattatagtgt

SEQ ID NO: 148 - DNA, 22 bp, Artificial Sequence
  Synthetic
ctcttccaaataatcctcaaaa

SEQ ID NO: 149 - DNA, 23 bp, Artificial Sequence
  Synthetic
gaatttgtaatttcgtgcgtttc

SEQ ID NO: 150 - DNA, 22 bp, Artificial Sequence
  Synthetic
aaaaaaaactctccgtacaccg

SEQ ID NO: 151 - DNA, 25 bp, Artificial Sequence
  Synthetic
tgaatttgtaattttgtgtgttttg

SEQ ID NO: 152 - DNA, 24 bp, Artificial Sequence
  Synthetic
aaaaaaaactctccatacaccacc

SEQ ID NO: 153 - DNA, 21 bp, Artificial Sequence
  Synthetic
atcgtcggggtttttttagtc

SEQ ID NO: 154 - DNA, 19 bp, Artificial Sequence
  Synthetic
aacaaaaacacgacccgac

SEQ ID NO: 155 - DNA, 23 bp, Artificial Sequence
  Synthetic
gtattgttggggtttttttagtt

SEQ ID NO: 156 - DNA, 21 bp, Artificial Sequence
  Synthetic
aaacaaaaacacaacccaacc

SEQ ID NO: 157 - DNA, 20 bp, Artificial Sequence
  Synthetic
ggtaggacgtttttagggtc

SEQ ID NO: 158 - DNA, 20 bp, Artificial Sequence
  Synthetic
atacaactctacacgacgca

SEQ ID NO: 159 - DNA, 23 bp, Artificial Sequence
  Synthetic
taaggtaggatgtttttagggtt

SEQ ID NO: 160 - DNA, 23 bp, Artificial Sequence
  Synthetic
aacatacaactctacacaacaca

SEQ ID NO: 161 - DNA, 21 bp, Artificial Sequence
  Synthetic
gtttaaaatgacgtaggcgtc

SEQ ID NO: 162 - DNA, 20 bp, Artificial Sequence
  Synthetic
aataccgcgaaactataccg

SEQ ID NO: 163 - DNA, 24 bp, Artificial Sequence
  Synthetic
ggtgtttaaaatgatgtaggtgtt

SEQ ID NO: 164 - DNA, 23 bp, Artificial Sequence
  Synthetic
acaataccacaaaactataccac

SEQ ID NO: 165 - DNA, 22 bp, Artificial Sequence
  Synthetic
ttttggttgttaggggtttatt

SEQ ID NO: 166 - DNA, 21 bp, Artificial Sequence
  Synthetic
acccacctactacrcaactac

SEQ ID NO: 167 - DNA, 36 bp, Artificial Sequence
  Synthetic
ggttaggtatagtggtttatatttgtaattttagta

SEQ ID NO: 168 - DNA, 33 bp, Artificial Sequence
  Synthetic
attaactaaactaatcttaaactcctaacctca

SEQ ID NO: 169 - DNA, 16 bp, Artificial Sequence
  Synthetic
cctaccttaacctccc

SEQ ID NO: 170 - DNA, 20 bp, Artificial Sequence
  Synthetic
cgaattatagcggcggaggt

SEQ ID NO: 171 - DNA, 19 bp, Artificial Sequence
  Synthetic
aaatcgcgtaaactccgcg

SEQ ID NO: 172 - DNA, 16 bp, Artificial Sequence
  Synthetic
cgttaggtcgggcggt

SEQ ID NO: 173 - DNA, 15 bp, Artificial Sequence
  Synthetic
gcgcgtacgcggagg

SEQ ID NO: 174 - DNA, 16 bp, Artificial Sequence
  Synthetic
cgacgacgaacgcgct

SEQ ID NO: 175 - DNA, 15 bp, Artificial Sequence
  Synthetic
cgggagggcgtgtga

SEQ ID NO: 176 - DNA, 18 bp, Artificial Sequence
  Synthetic
gaattcgttcgcgaggga

SEQ ID NO: 177 - DNA, 21 bp, Artificial Sequence
  Synthetic
aaacgaaccgcactcgttacc

SEQ ID NO: 178 - DNA, 15 bp, Artificial Sequence
  Synthetic
cgtcaccgacgcgaa

SEQ ID NO: 179 - DNA, 21 bp, Artificial Sequence
  Synthetic
cgcggtatagtttcgcggtat

SEQ ID NO: 180 - DNA, 19 bp, Artificial Sequence
  Synthetic
cgcgataacgaccgacaaa

SEQ ID NO: 181 - DNA, 17 bp, Artificial Sequence
  Synthetic
cgtagttcgcggtgagg

SEQ ID NO: 182 - DNA, 67 bp, Artificial Sequence
  Synthetic
tttattttttgtttttagggtgttgtttaaagtgtgtggagtaggtgttttgggtggagttataatt

SEQ ID NO: 183 - DNA, 66 bp, Artificial Sequence
  Synthetic
gttttaatttatttttcgtttttagggcgtcgtttaaagtgtgcggagtaggtgttttgggtggag

SEQ ID NO: 184 - DNA, 67 bp, Artificial Sequence
  Synthetic
gtgatcgttcgattttttatagcgatagcggcgtaggcgttttcgtattaatagttttttatttcgt

SEQ ID NO: 185 - DNA, 1500 bp, Homo sapiens
acacacacacacacagttcttttttttttttttttcctgagtctcgttctgtggcccacgctggagtgcagtggtgcgatcacatctcactgcagcctgaaactccctgactcaagcaatcctcccacctcagcctccagagtagctgggacttcagaagcacgcaaccacgccccactaatttttgtattttttgtagagacagggtttcacaatgctgcccaagctggtctcgaactcctgggctcaagctatcctaccgcgtcggtttccgaaagtgttgcgattacaggcgtgagccatagcgttacatgtggtgggggggaagagagagaaagagagagagtgagaagaaggaggaggaggaggaggaggagaaagaaggaagaaagaaaagaaaagaaatatagagagggtgttcaaatctactgttcaagtgctttccatgtacatataatctctttttaaaatcccatccagctctgttgagatgagaaaactgaagctctgagaggttgagtaactttatcacgctcccgcaaagataaaagaaccgccatctgcccatattaatttccctgagatgaaatgtgacagagaaagatacctcaaaattaaggaaatggagagcttcgagaatcatcttccatctccgccgagtcccccaggaacttggcccggagtctttgggctgtcccacctgatcttgccccagtgcggctccacaccctggcgggcggcgtacggagcgtcccggcccccggcaccagccagcgtcggagtgggtaggactggagatggcagcctccggtccaggggaagcgggactccacggaagggctgggaagagggtaggaagagggggcgggggcgggaactgttccgccccctccccctctgtctactttcattggccaagggcttcctggccccaacccacctcccgcctccagggcgccgcttaaagtgtgcggagcaggtgccttgggtggagccacaactttgcgcgagtgtcttggttgagcgcagcgcccattcattgcccgcgagcgtccatccatctgtccggccgactgtccagcgaaaggggctccaggccgggcgcagccgccacccgggggaccgaggccaggagaggggccaagagcgcggctgacccttgcgggccggggcaggggacggtggccgcggccatgcagtcctgtgccagggcgtgggggctgcgcctgggccgcggggtcgggggcggccgccgcctggctgggggatcggggccgtgctgggcgccgcggagccgggacagcagcagtggcggcggggacagcgccgcggctggggcctcgcgcctcctggagcgccttctgcccagacacgacgacttcgctcggaggcacatcggccctggggacaaagaccagagagagatgctgcagaccttggggctggcggtaaggacctccacccggccctccgcggcctcccctccgccctgcccgggct

SEQ ID NO: 186 - DNA, 1500 bp, Homo sapiens
tttcaccatgttggccaggctggtcttgaactcctgacctcgtgatccaccctccttggcctcccaaagttctgggattacaggagtgagccactgcgcccagccacatttaaattttagagtataattattgctgcttataattagaagtgagaagactgttttgaagtaagggacttccctggccaccctcgctaaaattgcaaccttaccctcactccatttctccttctctgctttatatttctccaaagcacttatatgataataaactgtataataccgtacttactgtgtttctcatgtatcccggctacaactgtcaactgcgtgggactggttttgtctgttttgttccctgctctatccctggagcgtaaagcagtgtctggtatagagtggatacttaatagatattcgttgaaccgggccagtggcggtggctcacgcctgtaatcccagcactttgggaggcggaggcaggcagatcacttgaggtcaggagttcgaggccagcctggccaacatggcgaaatcccgtctctactaaaaatacaaaaattagccgggtgttgtggtgcatccctgtaatcccagctacttcggaggctgaggcaggagaatcgcttgaacctgggaggcggacgttgcggtgagccgagatcgcgccattgtactccagcctgggcgacaaagagagacatcgactcaaaaaaaaaaaaattcgttgaacggaatgagtgaacaaacaaatggatggatgagtgaatgaatgaacgaaggaacaagtgaccgctcgaccttctacagcgacagcggcgcaggcgctcccgcaccaacagccccccaccccgccaccgccagagggcgcagacaggccgggtcccgagtgtctgtcccaggcccccgccccacaggatcccggcccgggagcccccgatgcagggacccgcccgggactctgggtccccgcccatacctcaacttcgcacgactgcgtgcctcaagccgacgcagcggcctactctcgcactgcagacggggaaactgaggcctgaggcggccggggtggggcagacctcccggcgagcccgagcccccgcccccggctagccccgccctggcccgtaagaagcacccggggcgcgaggcgaaggcgcacagcgcggggccaggctgggtccagcagcgcgatggcagctcagcggctgggcaagcgcgtgctgagcaagctgcagtctccatcgcgggcccgcgggccagggggcagtcccggggggctgcagaagcggcacgcgcgcgtcaccgtcaagtatgaccggcgggagctgcagcggcggctggacgtggagaagtggatcgacgggcgcctggaggagctgtaccgcggcatggtgagcccggggagggctcgggggtggtcggggacagatagagcccgggcgcggagaccccacgcccttggggtctcggtgcacactcgctggtgcacggccagcc

SEQ ID NO: 187 - DNA, 1500 bp, Homo sapiens
tcttcagaagtctagaactttaataaccttaaatccaaattattccctgcgtccatccataatgagaaggaagaaatctacacttgcatttcttccacctttcacatctttctcatttaaatcctactgtacttgatttgtgttgttcccaacgtactaggtgctatgcatgacgtagaaacgtgtttccttatatgtatgtactagcaggctccttcaagcacatgcctaaggttgaaagattcaaatataattatagcctcttaaattattgcgtcagagcccgccctatcccctcagaaaaagcccccttttacaccatcaaatgaaatatcaggttatttgcactggatattttttaaaagaaaatatttgattttaaaatcaaagagttcttggttttccatcatttcactaattgtcttactagagacccgagtctcctttgtcaaaatatatttcagtacgcattttaaaaaatatgtgtgtgtgtatacttaaatatatatatatataaagtcttggacttattttcacctagcctggggcatatcgtggatagtttcaatcgacaaaacaggcactctctggcctaggagtggaatccatcctcaattttcgcaggctcctcacaattctctatttggaagaagtgtccctctccttcccttttcttttcctcctttactcagcgtcagtccccgcagccatctcctccgaccctttttgtctacgtcccagcgtcgcgaaccacagcggcggaggtggagcggggagaggcgttaggccgggcggctaaaacgcgccgttaaagtgggggagagattgcgcggagcccacgcgatccctgggacgccggagacaacggggctcttgggaaggcgcggagcccggggaagccggggatgtgcgcgtgagccgtgcccgcagggtctccccgcctccgccacctttcttgggtggctctccgcctcgtcctccctccgagggccgttggtacattcctagtgactccaagcgcttaaaaggggcccgggaggatgaaccccacagatctgaacctgatttgtgtgtgcaccgcgtctccagcgatcccggatccactgcgctgccaggggcctgggggtgggtctcttgctgtctctgcgacgacatccttacgtttcggcactctaatgctgggtttgtgcgtgtgtgtctgcttagcggtctagcgggctgttaggctccctcgcccccagctccttggctcgctcagctcctccaccgcagcccagcagtgagacgcgcgcgcagccagctccccacgagatggaacagaccgaagtgctgaagccacggaccctggctgatctgatccgcatcctgcaccagctctttgccggcgatgaggtcaatgtagaggaggtgcaggccatcatggaagcctacgagagcgaccccaccgagtgggcaatgtacgccaagttcgaccagtacaggtgagcgcgctgcagcttc

SEQ ID NO: 188 - DNA, 1500 bp, Homo sapiens
tgtgtatttgggacaaacacaggttgtggacgacaggattgaaagtcagattaactggatcaaaattcaggctgcgtactgagccagtctaagatttcaccgaaatctgggagctgctcccactccctggacttttgtgcaaagactggaggaagctgtgaacagcctgggctaggagaacattccccctctgggaggagaggctcgcatgagctcatggggctttccaccgcagacccagccttggcagccacaaagtgtccagcagtgaaacctggcccttcagttctcaagggccctttggaacatatttgactctaagcagaggtcactattccaagagtgactcatgtcttggggttaagtggagatgatgggtgggatccatgaacagatccagctcttcccaatgtggggggcaccagagtgcatagcttgggagggttggtcatccgaagaggcactgcgtgggtgcatcccgggcaaaaaggatgagaaggtgatccactggcttccataccctgggaaaggtgtcagaccgtgaggtcacatcaaaaggtatgagtgtgggtacctccttccctcccccgacggtgggggcctcatctctcggccatatcacctctgtgccttgcacacttcctggctcagactcagccctgtccacgttctcttccaccatctgatctgcccccatctcccaaagactacttgtggggactagggaggtttaggggtgtagtcaagccagatgcaccgaggttgctgcggtgggaagagggcgctgggagggaggaggacctggggcgcagccgtggtgggtgcgccctgccaagaagggcgggggcgacggggcgcgcacgcggaggaggaggaggaggagggagacgggagggcgtgtgagcgagtgagacaagaaaagggagcgcgcccgccgccgccgccgccgccgccctcctctggagagagaggctggagtgaggctgtgcgaagcgccgcatttcaatgaggacgggccgaggcacatccctgcactagtggccgcaaccgaggcgccgcgctccagcagctgctgccgcccagcccggccccgccgccgccccccagccctgcagccccgcagccccggccgcgcccagcccggcgaggacagcaccaggaggcggcccccagcgcggccacaaagacccccggcggcgtctctccgcggaccggtgcgtggtttgccttccctggggacgggagctgcggggagggcggccgggggactccgcagagcccgctccgcccgcagccgcggggcgccgagggcagggcgggcgctcagcttggccccgccgccccgcccgcagttcctcgaaaggcggccgccctgccctggccagcctggcccgacccgggggcgttgcgtgcgggcggccccggggctgctggcacctgcctcttttgtttggtgccatggggctttaaacctggagtcaggcgagcgggccctcccag

SEQ ID NO: 189 - DNA, 1500 bp, Homo sapiens
tcggggctgtatttccaagaacgctctggaggcacagctcttttctaaggagggccatgttgcatgctgggaaatttttgaacttttcccaaatcacagcttttaagctttccgggcttattattattattattattattttgagacattgtttcactcttatcgcccaggctggagtgcagtggtgtgatctcggctcactgcaacctctgcctcctgggttcaagcgattctcttccctcagcctcccaagtagctgggattacaggcacacaccaccacgcctggctaatttttgcatttttagagcgatggggtttcaccatgttgggcaggagggtctcgaactcctgatctcaggtgatatacccgcctcggcctcccaaagtgctgagattacaggcgtgatttctggtcgtttcagactacatttccttcccagaaggacgcggggacacgccttccctatggacggatcccaaatgcaagttgtaagcaattataactgtaggcccctagagcctgaaccacttttcccaggaattttttgtggtgcgccttaggagcaagcaaagtttcagggcaggagaaggccgacagggcttccagcatttgaagattctgccagtctgaaatacgctttcccagaagtctatggggggtgcagctgctctaacagtgggaggagacaccgacgaaaagcattctgacggccgcgcttccagcgctaaggattcccgcgtagtgagcatttcccagaggtctctggggcatggctccattagggccgtgtgcagttgcctgcagggaaacactgatgggcgcccttccagatacgtcgattcccgggttatgagctacatttcccagaagccctcaaggcaggacgcccttagggtcgcgtagagttgcatgctgggaaatcctgacagccgcttccaactctgtggatttccgggttatggactacatttcccagaggcctctgggtcacggctccatttgcgccgtgtagagttgcatgctgggaaacactgacgggcgcccttccagctgcgtcgaagcttctctggtttcgcattacatttcccagagggccttggggcacagtccaatgaggagcgagccgggtggccggctgaggggcgtgcttcatgctcgcgggatttttaaaaatggtttctcctggatcttatcccaactgcgcctgagaccttgtttcacctcctgatgtgctgggtgtggttaggcctcgagccgggagctatttgcctcccaggaggaagccagggcagggttccgcggggaatggcggtgagggagccactcgggacgcggaggccctaaaatgaagaagcttcggcctgaggcttcgggagcggcgagcagggaagtgggagtgtgtgtgagtgtgtgtgtgtgtgtgtgtgtgtgtgtgtgaataagccagaggctgcctggtggggccccgccgtggcccagtgactgagataattgtct

SEQ ID NO: 190 - DNA, 1500 bp, Homo sapiens
ggccacatactgtatgatcccacttatatgacactgtgaaggcaaaattgtagcagtggactacttagattagctgttcctggggttatgggtggggagggagtgactaccaagaggcagcaggaggatttttgggcagtggagctattctttgtcccgagtttgttggtggatatgagttaaaattcataaaactgaataccaaaatgtgtggtgtatgtaaatttaaaatatataaactgttcagatatacattttaaaaatcagttcacacaaaaacgaccaaaatggcatcctcataagcgcctctatctggaaaactgaaatgggaagcagctcctggccctatcctgccttccaggggggctacatttatatggggctttgccaaagccctcaccccagctgccactcacatttccctcatttcagcagctcctgaatccctggggtgggtaatctttgagttctatccactccttgaaactgtagaacccattttagcgcatcagtgtctctgggtctccacctcttgtctccccagtactgggtctgctggtccctgtctctgtatctctgtccagatctgtctcagcctcgagtgctccttttctctgggtctctctcttttcctgagcctgagctggtctctttcttttcttgagcccatgctggtctttccctttcttgatcctgggctggtctccctcctttcttaaccgtgagctggtctctccctttccgtgtccgactctgtacatctctgtttcggggaccccagggcgtcttggtctgcccccgagcgtctgtgggccttcatgcttctctccctcccggtctgcgcgtctttttgtctttgtttctccgtttctcaccgtctttgtttctccgggtctctggctgtcaggggtctattatttacccgcttgtttacttcgttgccatggtgcccaaaatgacgcaggcgccggagtgggcgggacgccgggaggccacactcgcgtctgattggccatgcgcactgtcacctgtcacggctcccttgctcgccccggtgcgcggcacagcctcgcggcattgtgggagtcgtagtccgcggtgaggcccagtgcgcaggcgcagtttgccggccgccatcgcgcactggggctccgggcggctgggaccggcctggggtagctgcgcagcaggtgggtcggtcccggggatgcagcgcgggggtcagagagcgaccccaacagggtagacggtgaggacgcctcagtgggcagcgttagggaaaggggccagggtgttcccggggttggggtgcgagcccccgctgtgcgtccaggaggccgagggtctggcatcccttcgcgtggagtgcggggcaggggccgggtccccccggaggtggctgagatccagatcggtgaggggccctgcaggcaggtagggaactagtgcaacccccaccccgtgtaggctcgtggaggggaggcgcttagggcaccctgt